(19) United States
(12) Patent Application Publication
(10) Pub. No.: US 2010/0041622 A1
(43) Pub. Date: Feb. 18, 2010

(54) Compositions containing aminoalkanes and aminoalkane derivatives

(76) Inventors: Philip J. Bromley, Fullerton, CA (US); Paul Edelmann, Hollywood, FL (US)

Correspondence Address: K&L Gates LLP, 3580 Carmel Mountain Road, Suite 200, San Diego, CA, 92130, US

(21) Appl. No.: 12/583,209

(22) Filed: Aug. 13, 2009

Related U.S. Application Data
(60) Provisional application No. 61/188,900, filed on Aug. 13, 2008.

(51) Int. Cl. A61K 31/714; A61K 31/661; A61K 31/13

(52) U.S. Cl. 514/52; 514/121; 514/663

(57) ABSTRACT

Provided are palatable liquid compositions that contain 2-aminoalkanes that have vasoconstrictor activity, and other additives, including taste-modifying agents. Also provided are methods for making and using the compositions to provide stimulant activity for increasing energy, alertness, endurance, and/or any other consequent physical manifestation of the vasoconstrictor activity.

### RELATED APPLICATIONS

Benefit of priority under 35 U.S.C. §119(e) is claimed to U.S. Provisional Application Ser. No. 61/188,900, to Philip Bromley and Paul Edelmann, entitled, “COMPOSITIONS CONTAINING AMINOALKANES AND AMINOALKANE DERIVATIVES,” filed Aug. 13, 2008.

This application is related to International Application No. (Attorney Dkt. No. 0119360-00101/5717PC), filed Aug. 13, 2009, entitled “COMPOSITIONS CONTAINING AMINOALKANES AND AMINOALKANE DERIVATIVES,” which also claims priority to U.S. Provisional Application Ser. No. 61/188,900.

The subject matter of each of the above-referenced applications is incorporated by reference in its entirety.

### FIELD OF THE INVENTION

Provided are compositions, including low-volume palatable compositions, containing active ingredients, such as vasoconstrictors, including aminoalkanes such as 2-aminoalkanes, and other additives, and methods for making and using the compositions, such as for delivery of the active ingredients to a subject.

### BACKGROUND

Vasoconstrictors, also called vasopressors or simply pressors, are compounds that cause vasoconstriction (narrowing of blood vessels) when administered to subjects such as animals and humans. Vasoconstrictors can cause other biologic effects, such as stimulatory and other sympathomimetic effects, decongestion (e.g. bronchial or nasal decongestion), increased energy, endurance, mood-enhancement, appetite suppression and/or weight loss and other effects. Vasoconstrictors include alpha-adrenergic agonists, such as some aminoalkanes, such as 2-aminoalkanes and derivatives thereof, such as aldehydes, carbonates and acid addition salts (see, for example, U.S. Pat. Nos. 2,350,318, 2,386,273 and 2,455,193; and Swanson and Chen, Journal of Pharmacology and Experimental Therapeutics, 88(1), 10-13 (1946)).

These compounds can provide benefits when provided orally. There, however, is a need for palatable compositions containing effective amounts of biocompatible aminoalkanes. Accordingly, it is among the objects herein to provide such compositions.

### SUMMARY

Provided are compositions containing an aminoalkanes or a derivative thereof. The aminoalkanes or derivatives thereof have vasoconstrictor activity and are intended for human consumption. 2-amino-4-methylhexane HCl and derivatives thereof are exemplary aminoalkanes for use in the compositions provided. The compositions as provided are low volume, such as about or 4 ml or less, liquid compositions that contain a high concentration, such as, as high or higher than 200 mM of the aminoalkane, for ingestion by a subject, particularly a human. The compositions can act as stimulants to increase energy, alertness, endurance, and/or any other consequent physical manifestation of the vasoconstrictor activity. The compositions can further contain one or more additional ingredients, such as, but not limited to, surfactants, nutritional supplements, vitamins, minerals, fatty acids, and weight-loss compounds, including, but not limited to appetite suppressants, fat and/or starch absorption blocking compounds, compounds that increase metabolism or any additional active ingredient that increases energy, promotes weight loss and/or promotes health. The one or more additional active ingredients can be added alone or in any combination so as to produce a desired effect, such as increasing energy, promoting weight loss and/or promoting health. The compositions are formulated so that they are palatable when consumed as a liquid with the high concentration of aminoalkane. They can be provided in small containers, such as ampoules, for convenient administration. The compositions, which contain ingredients that are water soluble and ingredients that are fat or oil soluble can be formulated as emulsions.

The compositions are formulated for oral ingestion and can include taste-modifying agents, e.g. flavoring agents, sweetening agents and other agents, that render them palatable. As noted, in particular, low volume compositions with high concentrations of the aminoalkanes are provided. For example, included are compositions having a total volume that is less than or equal to at or about 500 mL, 400 mL, 300 mL, 240 mL, 200 mL, 150 mL, 120 mL, 100 mL, 50 mL, 10 mL, 5 mL or 4 mL, and compositions having a volume of 10 mL, 9 mL, 8 mL, 7 mL, 6 mL, 5 mL, 4 mL, 3 mL, 2 mL or 1 mL, generally containing as much as a 100 mM-200 mM concentration of the aminoalkane. Typically, the compositions are liquid compositions, typically liquid aqueous compositions or emulsions, formulated for single dosage administration and provided in amounts of 5 ml or less containing as much as 100 mM-200 mM, particularly, 100-150 mM, such as 100-110 mM, of the amino alkane.

The compositions contain aminoalkane and/or aminoalkane derivative compounds of formula I:

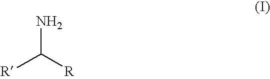

and biocompatible derivatives thereof, where one of R and R′ is an alkyl containing from 2 to 20 carbons, such as 2-10, 2-8, 2-7, 2-6, 2-5, 2-4 and 2-3 carbons and the other is a hydrogen or an alkyl containing from 1-20 carbons, such as 1-10, 1-8, 1-7, 1-6, 1-5, 1-4, 1-3 and 1 or 2 carbons. The compositions contain taste-modifying agents to render the compositions palatable, e.g. increase or enhance palatability compared to the composition in the absence of the taste-modifying agents, for oral ingestion by a subject. The aminoalkanes and derivatives thereof in the compositions are any that have vasoconstrictor activity.

2-amino-4-methylhexane HCl and its derivatives are among well known aminoalkanes with vasoconstrictor activity. 2-amino-4-methylhexane HCl is exemplified herein. Compositions containing other aminoalkanes with such activity can be prepared in accord with the description herein by normalizing the amount of aminoalkane or derivative thereof to have the same vasoconstrictor activity as the specified amount of 2-amino-4-methylhexane HCl so that the composition delivers the same amount of activity. Thus, a compound that that has about half the activity of 2-amino-4-methylhexane HCl can be included in the composition at twice the concentration.

The amount of the aminoalkane or derivative thereof is typically the highest possible amount of the aminoalkane (or the amount providing the highest amount of vasoconstrictor activity), or the amount closest to the desired amount of activity, such that the resulting composition, when formulated for single dosage administration, is a palatable composition and delivers the desired effect. The concentration of aminoalkane is typically between at or about 3 mM and at or about 1000 mM, for example, at or about 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 155, 160, 165, 170, 175, 180, 185, 190, 195, 196, 197, 198, 199, 200, 210, 220, 230, 240, 250, 260, 270, 280, 290, 300, 350, 400, 500 or 1000 mM, typically between at or about 3 mM and at or about 500 mM, such as between at or about 3 mM and at or about 250 mM or between at or about 3 mM and at or about 200 mM, and typically between at or about 3 mM and at or about 150 mM, typically between at or about 3 mM and at or about 120 mM, such as between at or about 3 mM and at or about 10 mM, e.g. between at or about 100 mM and at or about 110 mM, e.g. at or about 107 mM; or between at or about 1 mg/mL and at or about 100 mg/mL, typically between at or about 1 mg/mL and 50 mg/mL or between at or about 1 mg/mL and 35 mg/mL, e.g. at or about 1, 2, 3, 4, 5, 6, 7, 8, 9, 10, 11, 12, 13, 14, 15, 16, 16.25, 16.5, 16.75, 17, 18, 19, 20, 21, 22, 23, 24, 25, 26, 27, 28, 29, 30, 31, 32, 33, 34 or 35 mg/mL, and can be determined empirically, by testing various amounts for palatability.

For example, provided are compositions containing the aminoalkane or derivative thereof at an amount (concentration) in the composition between at or about 3 mM and at or about 250 mM, or between at or about 40 mM and at or about 250 mM, or between at or about 3 mM and at or about 500 mM; compositions containing the aminoalkane or derivative at an amount in the composition that is at or about 3 mM, 5 mM, 10 mM, 15 mM, 20 mM, 25 mM, 30 mM, 35 mM, 40 mM, 45 mM, 50 mM, 55 mM, 60 mM, 65 mM, 70 mM, 85 mM, 90 mM, 100 mM, 101 mM, 102 mM, 103 mM, 104 mM, 105 mM, 106 mM, 107 mM, 108 mM, 109 mM, 110 mM, 115 mM, 120 mM, 130 mM, 140 mM, 150 mM, 160 mM, 170 mM, 180 mM, 190 mM, 200 mM, 210 mM, 220 mM, 230 mM, 240 mM or 250 mM; compositions containing the aminoalkane or derivative thereof at between at or about 100 mM and at or about 110 mM; and compositions containing the aminoalkane or derivative thereof at a concentration in the composition of at or about 107 mM or between at or about 1 mg/mL and at or about 50 mg/mL, compositions containing the aminoalkane or derivative thereof at a concentration of between at or about 1 mg/mL and at or about 25 mg/mL, and compositions containing the aminoalkane or derivative thereof at a concentration of at or about 1, 2, 3, 4, 5, 6, 7, 8, 9, 10, 11, 12, 13, 14, 15, 16, 16.25, 16.5, 16.75, 17, 18, 19, 20, 21, 22, 23, 24, 25, 30, 35, 40, 45 or 50 mg/mL. to deliver an effective amount of the aminoalkane to increase energy or alertness by virtue of the vasoconstrictor activity when formulated for single dosage administration. Generally single dosage administration is between about 1 and 10 mL, such as 4 or 5 mL, of the composition.

The aminoalkanes and aminoalkane derivatives in the compositions also can be amounts having the same activity, typically vasoconstrictor activity, as a specified amount (e.g. mass or concentration) of 2-amino-4-methylhexane HCl or other standard compound. For example, the provided compositions include compositions containing an amount of aminoalkane or derivative thereof that has the same (equivalent) vasoconstrictor activity as 2-amino-4-methylhexane HCl at a concentration of a specified amount, such as between at or about 3 mM and at or about 1000 mM, such as at or about 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 155, 160, 165, 170, 175, 180, 185, 190, 195, 196, 197, 198, 199, 200, 210, 220, 230, 240, 250, 260, 270, 280, 290, 300, 350, 400, 500 or 1000 mM, and typically between at or about 3 mM and at or about 250, or between at or about 3 mM and at or about 200 mM, for example, such as between at or about 5 mM and 150 mM, e.g. between at or about 25 mM and 110 mM, e.g. at or about 107 mM, or between at or about 1 mg/mL and at or about 50 mg/mL, or between at or about 1 mg/mL and at or about 25 mg/mL 2-amino-4-methylhexane HCl. In another example, the concentration of aminoalkane or derivative thereof is such that the vasoconstrictor activity is equivalent to at or about 1, 2, 3, 4, 5, 6, 7, 8, 9, 10, 11, 12, 13, 14, 15, 16, 16.25, 16.5, 16.75, 17, 18, 19, 20, 21, 22, 23, 24, 25, 30, 35, 40, 45 or 50 mg/mL 2-amino-4-methylhexane HCl.

For example, provided are compositions where the concentration of the aminoalkane or derivative is such that it has the same vasoconstrictor activity as 2-amino-4-methylhexane HCl, at a concentration of between at or about 3 mM and at or about 500 mM. Also provided are compositions with a concentration of aminoalkane or aminoalkane derivative that has the same vasoconstrictor activity as 2-amino-4-methylhexane HCl at a concentration of between at or about 3 mM and at or about 250 mM, or between at or about 40 mM and at or about 250 mM. In another example, the concentration of the aminoalkane or derivative thereof is such that it has the same vasoconstrictor activity as 2-amino-4-methylhexane HCl at a concentration of at or about 3 mM, 5 mM, 10 mM, 15 mM, 20 mM, 25 mM, 30 mM, 35 mM, 40 mM, 45 mM, 50 mM, 55 mM, 60 mM, 65 mM, 70 mM, 85 mM, 90 mM, 100 mM, 101 mM, 102 mM, 103 mM, 104 mM, 105 mM, 106 mM, 107 mM, 108 mM, 109 mM, 110 mM, 115 mM, 120 mM, 130 mM, 140 mM, 150 mM, 160 mM, 170 mM, 180 mM, 190 mM, 200 mM, 210 mM, 220 mM, 230 mM, 240 mM or 250 mM. In another example, the concentration of aminoalkane or derivative thereof is such that it has the same vasoconstrictor activity as 2-amino-4-methylhexane HCl at a concentration of between at or about 100 mM and at or about 110 mM. In another example, the concentration of aminoalkane or derivative thereof is such that it has equivalent vasoconstrictor activity to a concentration of at or about 107 mM 2-amino-4-methylhexane HCl or is at or about 107 mM. In another example, the concentration of aminoalkane or derivative thereof is such that the vasoconstrictor activity is equivalent to between at or about 1 mg/mL and at or about 50 mg/mL, or between at or about 1 mg/mL and at or about 25 mg/mL 2-amino-4-methylhexane HCl. In another example, the concentration of aminoalkane or derivative is such that the vasoconstrictor activity is equivalent to at or about 1, 2, 3, 4, 5, 6, 7, 8, 9, 10, 11, 12, 13, 14, 15, 16, 16.25, 16.5, 16.75, 17, 18, 19, 20, 21, 22, 23, 24, 25, 30, 35, 40, 45 or 50 mg/mL 2-amino-4-methylhexane HCl.

The aminoalkanes and derivatives thereof include any aminoalkanes and their derivatives that have vasoconstrictor activity, particularly those having similar activity compared to 2-amino-4-methylhexane HCl. The aminoalkanes and derivatives include compounds (and derivatives) of Formula I, above, such as 2-aminoalkanes and their derivatives. The aminoalkanes and derivatives include aminoalkanes containing between 4 and 20 carbon atoms, typically between 4 and 9 carbon atoms or between 6 and 9 carbon atoms, and derivatives of such aminoalkanes. The aminoalkanes and derivatives include aminoalkanes containing substitutions, e.g. methyl substitutions, for example, methyl substitutions on the fourth carbon, such as 2-amino-4-methylhexane and other aminoalkanes methylated on the fourth carbon, and their derivatives.

Exemplary of the aminoalkanes and derivatives are aminoalkanes of formula I including those having straight or branched R and/or R′ alkyl groups. For example, straight chain alkyl groups, including but not limited to, methyl, ethyl, propyl, n-butyl and n-pentyl; and/or branched alkyl groups, including but not limited to, isopropyl, t-butyl and 2-methylbutyl; and particularly 2-aminoalkanes, such as branched and substituted 2-aminoalkanes, and analogs and derivatives thereof, including biocompatible salts, aldehydes, amides thereof, carbonate derivatives, and ester and acid derivatives thereof, particularly those compounds and derivatives having vasoconstrictor activity that is equivalent or about equivalent to that of 2-amino-4-methylhexane and/or salts of 2-amino-4-methylhexane, such as 2-amino-4-methylhexane HCl or a specified amount of 2-amino-4-methylhexane HCl.

The 2-aminoalkanes and derivatives include 2-aminoalkanes having between 3 and 22 carbon atoms, such as between 3 and 20, and typically between 3 and 15 or 3 and 10, such as 3, 4, 5, 6, 7, 8 or 9 carbon atoms, typically between 4 and 9 or 6 and 9 carbon atoms, particularly those compounds having vasoconstrictor activity that is equivalent or about equivalent to that of 2-amino-4-methylhexane and/or salts of 2-amino-4-methylhexane, such as 2-amino-4-methylhexane HCl or a specified amount of 2-amino-4-methylhexane HCl, including 2-aminoalkanes having between 4 and 9 carbon atoms, typically 2-aminoalkanes having between 6 and 9 carbon atoms, 2-aminoalkanes having between 3 and 22 carbon atoms, such as between 3 and 20, between 3 and 15, or between 3 and 10 carbon atoms, and typically between 4 and 9 carbon atoms, or between 6 and 9 carbon atoms, such as 2-aminoalkanes containing one or more methyl groups, such as, but not limited to, 2-amino-4-methylhexane (also known as 1,3-dimethylamylamine, 1,3-dimethylpentylamine, 2-amino-4-methylhexane, 4-methyl-2-hexylamine, methylhexaneamine, AMH, Forthane and 4-methyl-2-hexanamine), 2-aminoheptane, 2-amino-4-methylheptane, 3-methylhexan-2-amine, 5-methylhexan-2-amine, 3-ethylhexan-2-amine, 5-methylhexan-2-amine and 4-methylhexane-2,5-diamine, and analogs and derivatives thereof, including biocompatible salts, aldehydes and amides thereof, including carbonate derivatives and ester and acid derivatives thereof.

In particular, the aminoalkanes and derivatives include 2-amino-4-methylhexane and acid salts and derivatives thereof, such as 2-amino-4-methylhexane HCl and other acid salts having equivalent or about equivalent vasoconstrictor activity, such as 2-amino-4-methylhexane HCl. Exemplary of the aminoalkane derivatives are aldehyde derivatives, such as, but not limited to, aldehydes of 2-aminoalkane, such as, but not limited to, 2-benzalaminohexane, 2-propionalaminoheptane, 2-ethanalaminoheptane, 2-propionalamino-4-methylhexane, 2-benzalamino-5-methylhexane, 2-isobutanol-amino-4-methylheptane, 2-propionalamino-6-methylheptane, 2-propionalamino-4,6,-dimethylheptane and 2-propionalamino-4-methylhexane.

The aminoalkane derivatives also include biocompatible salts of aminoalkanes (e.g. 2-aminoalkanes), such as 2-amino-4-methylhexane HCl and salts having a comparable vasoconstrictor activity to 2-amino-4-methylhexane HCl, such as salts formed by treatment of aminoalkanes with an organic acid, such as acetic acid, adamantanecarboxylic acid, adipic acid, ascorbic acid, aspartic acid, azelaic acid, benzoic acid, 2-(4-hydroxybenzoyl)benzoic acid, carboxylic acid, cinnamic acid, citric acid, cyclohexanecarboxylic acid, decanoic acid, dodecanoic acid, 1,2-ethanedisulfonic acid, ethanesulfonic acid, ethylenediaminetetraacetic acid (EDTA), fumaric acid, glucoheptonic acid, gluconic acid, glutamic acid, glycolic acid, hippuric acid, lactic acid, lactobionic acid, maleic acid, hydroxymaleic acid, methylmaleic acid, malic acid, malonic acid, mandelic acid, 1-hydroxy-2-naphthoic acid, 3-hydroxy-2-naphthoic acid, octanoic acid, pamoic acid, pantothenic acid, phthalic acid, phenylacetic acid, pimelic acid, phosphonic acid, propionic acid, 2-(4-chlorophenoxy)-2-methylpropionic acid, salicylic acid, 4-aminosalicylic acid, stearic acid, suberic acid, succinic acid, sulfamic acid, N-cyclohexylsulfamic acid, N-methyl-, N-ethyl-, or N-propyl-sulfamic acid, sulfonic acid, benzenesulfonic acid, disulfonic acid, ethane-1,2-disulfonic acid, 2-hydroxyethanesulfonic acid, methane- or ethane-sulfonic acid, 2-, 3- or 4-methylbenzenesulfonic acid, 2-naphthalenesulfonic acid, 1,5-naphthalene-disulfonic acid, p-toluenesulfonic acid, methylsulfuric acid, ethylsulfuric acid, dodecylsulfuric acid, tannic acid, tartaric acid, terephthalic acid, and 10-undecenoic acid and other organic protonic acids; and inorganic acid addition salts of aminoalkanes, such as, but not limited to, bicarbonates, carbonates, chlorides, bromides, iodides, nitrates, perchlorates, phosphates, monohydrogenphosphates, dihydrogenphosphates, metaphosphates, pyrophosphates, sulfates, pyrosulfates, bisulfates, sulfites, bisulfites, hydrochloric acid addition salts, sulfuric acid addition salts, and hydrobromic acid addition salts. Thus, exemplary aminoalkane derivatives of the compositions include, but not limited to, acetic acid addition salts, hydrobromic acid addition salts, sulfuric acid addition salts, maleic acid addition salts, propionic acid addition salts and malonic acid addition salts and hydrochloric acid addition salts, including, but not limited to, 2-amino-4-methylhexane HCl (also called 1,3-dimethylamylamine HCl, 1,3-dimethylpentylamine HCl, 2-amino-4-methylhexane HCl, 4-methyl-2-hexylamine HCl, methylhexaneamine HCl, AMH HCl, Forthane HCl and 4-methyl-2-hexanamine HCl).

The derivatives also include carbonates derivatives of aminoalkanes, such as of 2-aminoalkanes, particularly carbonate derivatives of 2-amino-4-methylhexane and carbonate derivatives having equivalent or about equivalent vasoconstrictor activity, and include, but are not limited to, 2-aminoheptane carbonate, 2-amino-4-methylhexane carbonate and 2-amino-4-methylheptane carbonate. The aminoalkane derivatives include 2-amino-4-methylhexane HCl derivatives and analogs thereof that have vasoconstrictor activity.

For example, the aminoalkanes and biocompatible derivatives include compounds having equivalent or about equivalent vasoconstrictor activity (per molecule or per mass), compared to 2-amino-4-methylhexane HCl. Vasoconstrictor activity can be determined by known methods, such as those as described herein.

The taste-modifying agents include flavoring agents, such as flavoring agents that confer fruit, mint, cinnamon and other flavors, such as peach, sour apple, mint, peppermint, spearmint and other flavors. The taste-modifying agents further can include sweetening agents (sweeteners), such as sugar, and typically sugar substitutes, such as aspartame, saccharin, sucralose, neotame, and acesulfame potassium and other sugar substitutes. The amount of the taste-modifying agents typically is an amount to render the composition palatable, e.g. to improve or enhance the palatability of the composition, compared with the absence of the taste-modifying agents, and can be determined empirically. In one example, the amount of the taste-modifying agent is in the composition at an amount between at or about 0.1% and at or about 25%, or between at or about 0.45% and at or about 3%, by weight. In another example, the amount of the taste-modifying agent in the composition is at or about 0.45%, 0.5%, 0.6%, 0.7%, 0.8%, 0.9%, 1%, 1.1%, 1.2%, 1.3%, 1.4%, 1.44%, 1.5%, 1.6%, 1.7%, 1.8%, 1.9%, 2.0%, 2.1%, 2.3%, 2.4%, 2.5%, 2.6%, 2.7%, 2.8%, 2.9% or 3%, by weight, of the composition.

In one example, the amount of the flavoring agent in the composition is between at or about 0.1% and at or about 25%, or between at or about 0.45% and at or about 1.5%, by weight. In another example, the amount of the sweetening agent, e.g. the sugar or sugar substitute, in the composition is between at or about 0.1% and at or about 25%, by weight, of the composition, or between at or about 1.4% and at or about 25%, by weight, of the composition.

The compositions can further contain preservatives, such as preservatives in amounts sufficient to preserve or stabilize the compositions. The preservatives include sodium benzoate and potassium sorbate and other preservatives, such as those described herein. In one example, the preservative is in the composition at an amount of between at or about 0.1% and at or about 25%, or between at or about 0.4% and at or about 0.8%, by weight, of the composition.

The compositions can further contain one or more additional active ingredients, such as, but not limited to, nutritional supplements, vitamins, minerals, fatty acids, appetite supressants and weight-loss compounds, for example, caffeine, caffeine anhydrous, Vitamin B12, chromium picolinate, L-taurine or other amino acid (free or conjugated or combined with others), particularly those known to promote weight loss, alpha lipoic acid, R-lipoic acid, N-acetyl carnitine, L-carnitine, beta-alanine, glycine, carnitine and/or conjugated linoleic acid (CLA). The amount of the additional active ingredients typically is an amount sufficient to confer an effect, such as a biologic effect to a subject upon administration, and where the composition remains palatable. In one example, the amount of the one or more additional active ingredients in the composition, individually or in combination, is between at or about 0.01% and at or about 25%, by weight, or between at or about 0.015% and at or about 5%, by weight. In one example, the compositions contain caffeine anhydrous, at a concentration of at or about 2%, by weight. In another example, the composition contains Vitamin B12, at an amount of at or about 0.025%, by weight, of the composition. In another example, the composition contains chromium picolinate at an amount of at or about 0.015%, by weight, of the composition. In another example, the composition contains L-taurine, at an amount of at or about 0.25%, by weight, of the composition. In another example, the composition contains alpha lipoic acid, at an amount of at or about 0.025%, by weight, of the composition. In another example, the composition contains CLA, at an amount of at or about 0.248%, by weight, of the composition.

The compositions further can include surfactants to increase stability of the emulsion. Of particular interest are surfactants that results in a clear, rather than cloudy composition. Such surfactants, as described herein, include PEG-derivative of Vitamin E or similar surfactant, such as one having a similar or the same HLB value. Exemplary of the PEG-derivative of Vitamin E surfactants are tocopherol polyethylene glycol diesters (TPGDs), tocopherol sebacate polyethylene glycol, tocopherol dodecanedioate polyethylene glycol, tocopherol suberate polyethylene glycol, tocopherol azelaate polyethylene glycol, tocopherol citraconate polyethylene glycol, tocopherol methylcitraconate polyethylene glycol, tocopherol itaconate polyethylene glycol, tocopherol maleate polyethylene glycol, tocopherol glutarate polyethylene glycol, tocopherol glutaconate polyethylene glycol and tocopherol phthalate polyethylene glycol, and particularly, tocopherol polyethylene glycol succinate (TPGS), such as TPGS-1000 and d-α TPGS, e.g. d-α TPGS 1000, and analogs and homologs of TPGS, such as homologs that differ from TPGS parent compounds by one or more methylene unit(s). In one example, the PEG moieties in the surfactants are methylated PEG (m-PEG), PEG-OH, PEG-NHS, PEG-aldehyde, PEG-SH, PEG-NH₂, PEG-CO₂H, or branched PEGs. In one aspect, the PEG moiety has a molecular weight of between 200 or about 200 to 20,000 or about 20,000 KDa, between 200 or about 200 and 6000 or about 6000 KDa, between 600 or about 600 KD and 6000 or about 6000 KDa, between 200 or about 200 KD and 2000 or about 2000 KD, between 600 or about 600 Kd and 1500 or about 1500 KD, or between 600 or about 600 and 1000 or about 1000 KDa.

The amount of the surfactant can be between at or about 0% and 50%, by weight, and typically is between at or about 0.1% and at or about 25%, such as at or about 0.1%, 0.2%, 0.3%, 0.4%, 0.5%, 0.6%, 0.61%, 0.62%, 0.625%, 0.63%, 0.64%, 0.65%, 0.66%, 0.67%, 0.68%, 0.69%, 0.7%, 0.71%, 0.72%, 0.73%, 0.74%, 0.75%, 0.76%, 0.77%, 0.78%, 0.79%, 0.8%, 0.9%, 1.0%, 1.1%, 1.2%, 1.25%, 1.3%, 1.35%, 1.4%, 1.45% or 1.5%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24% or 25%, such as between at or about 0.1% and at or about 5%, or between at or about 0.1% and at or about 2%, or between at or about 0.6% and at or about 1.3%, e.g. between at or about 0.625% and at or about 1.5%. In one example, the amount of surfactant is less than 5%, by weight, of the composition, e.g. between at or about 0.01% and 5%, or between at or about 0.01% and 1.5%, e.g. at or about 1.5%, 1.4%, 1.3%, 1.25%, 1.15%, 1%, 0.9%, 0.8%, 0.75%, 0.7%, 0.675%, 0.65%, 0.625%, e.g. between at or about 0.625% and at or about 0.75% or between at or about 0.625% and at or about 1.5%, by weight of the composition. In one example, the amount of the surfactant is between at or about 0.1% and at or about 1%, by weight. In another example, the amount of the surfactant is between at or about 0.625% and at or about 0.75%, by weight, of the composition.

The provided compositions can contain a co-surfactant. Exemplary of co-surfactants are phospholipids, such as a phosphatidylcholine and compositions containing phosphatidylcholine. The amount of co-surfactant in the composition can be between at or about 0% and at or about 50%, by weight, and typically is between at or about 0% and at or about 25%, by weight, e.g. at or about 0.01%, 0.02%, 0.03%, 0.04%, 0.05%, 0.06%, 0.07%, 0.075%, 0.08%, 0.085%, 0.09%, 0.095%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, %, 11%, 12%, 13%, 14%, 15%, 16%, 17%, 18%, 19%, 20%, 21%, 22%, 23%, 24% or 25%, such as between at or about 0% and 1%, or between at or about 0% and 0.1%, e.g. between at or about 0% and 0.075%, such as at or about 0.075%. In one example, the amount of phospholipid (e.g. phosphatidylcholine) is less than 1% or less than 0.1%, by weight, of the composition, and typically is less than the amount of the surfactant. In one example, the amount of co-surfactant is between at or about 0.01% and at or about 25%, by weight, of the composition. In another example, the amount of the co-surfactant is at or about 0.075%, by weight, of the composition.

The provided compositions can contain an emulsion stabilizer, typically in an amount sufficient to stabilize the composition compared to the absence of the emulsion stabilizer. Exemplary of the emulsion stabilizers include, but are not limited to, compounds containing a blend of gums, for example, compounds containing a blend of any one or more of guar gum, xanthan gum and sodium alginate. The amount of the emulsion stabilizer in the composition typically is an amount sufficient to stabilize the composition compared to the composition in the absence of the emulsion stabilizer, and can be determined empirically and can be between at or about 0% and at or about 50%, and typically between at or about 0% and at or about 25%, by weight, e.g. at or about 0.01%, 0.02%, 0.03%, 0.04%, 0.05%, 0.06%, 0.07%, 0.08%, 0.09%, 0.1%, 0.12%, 0.13%, 0.14%, 0.15%, 0.16%, 0.165%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, by weight, such as between at or about 0.01% and 5%, or between at or about 0.1% and 1%, by weight. The amount of the emulsion stabilizer in the composition can be between at or about 0.01% and at or about 25%, by weight, an amount between at or about 0.1% and at or about 0.2%, by weight, of the composition. In one example, the amount of the emulsion stabilizer in the composition is at or about 0.165%, by weight, of the composition.

The compositions typically contain water, such as water at an amount sufficient to render the compositions aqueous or water soluble. The water typically is purified water, such as city water that is purified according to the provided methods described herein. The amount of water can be any amount sufficient to render the composition aqueous or hydrophilic, and typically is low enough such that the composition is a low-volume composition, such as, for example, compositions containing 10, 9, 8, 7, 6, 5, 4, 3, 2, or 1 mL. The amount of water in the composition can be between at or about 0% and at or about 99%, by weight, or between at or about 0.01% and at or about 95%, and typically is between at or about 10% and at or about 95%, by weight, such as at or about 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 89.1%, 89.2%, 89.3%, 89.4%, 89.465%, 89.47%, 89.5%, 89.6%, 89.7%, 89.8%, 89.9%, 89.965%, 90%, 90.1%, 90.2%, 90.3%, 90.388%, 90.39%, 90.4%, 90.5%, 90.515%, 90.52%, 90.6%, 90.7%, 90.8%, 90.9%, 91%, 91.1%, 91.2%, 91.3%, 91.332%, 91.4%, 91.5%, 91.6%, 91.7%, 91.8%, 91.8568%, 91.86%, 91.9%, 92%, 93%, 94% or 95%, and in some examples is greater than at or about 70%, by weight, of the composition, such as between at or about 70% and at or about 95%, by weight, of the composition, e.g. between at or about 85% and at or about 95%, such as between at or about 89% and at or about 92%, by weight or between at or about 89% and at or about 91%, by weight, of the composition. In some examples, the amount of water is an appropriate amount of water that conveys an aqueous composition. In some cases, the amount of water is selected according to the desired amounts (e.g. concentrations) of the other ingredients of the composition, for example, enough water to dissolve the desired amount of the aminoalkane or other active ingredient. In some examples, water is added to bring the composition up to the desired volume, e.g. low volume, such as between at or about 1 mL and at or about 10 mL, e.g. 1, 2, 3, 4, 5, 6, 7, 8, 9 or 10 mL. In such an example, the water can be added during the water phase production and/or after combining the water and oil phases of the composition, according to the provided methods for making the compositions. Exemplary of the amounts of water in the compositions are at or about 89%, 90%, 91% or 92%, by weight, of the composition.

Typically, the compositions contain pH adjusting agents, typically at an amount sufficient to change the pH of the composition, such as to raise or lower the pH. Exemplary of the pH adjusting agents are bases and acids, such as, but not limited to, acetic acid. The amount of the pH adjusting agent typically is the amount sufficient to render the composition a particular pH, for example, a pH between at or about 2 and at or about 5, such as between at or about 2 and at or about 4, between at or about 2 and at or about 3.5, or between at or about 2 and at or about 3.3, and typically is between at or about 0% and at or about 5%, by weight, such as between at or about 0.1 and 5%, e.g. at or about 0.1, 0.2, 1, 2, 2.5, 2.6, 2.7, 3, 4 or 5%, by weight, of the composition. In one example, the amount of the pH adjusting agent in the composition is at or about 0.17%, by weight, of the composition; in another example, it is at or about 2.66%, by weight, of the composition.

In one example, a particular ingredient can be used a preservative, a surfactant, a co-surfactant, an emulsion stabilizer, or a pH adjusting agent.

In a particular embodiment, provided are compositions containing 2-amino-4-methylhexane HCl having vasoconstrictor activity, taste-modifying agents including sucralose and a peach flavoring agent, caffeine anhydrous, conjugated linoleic acid, phosphatidylcholine, a TPGS surfactant, an emulsion stabilizer containing guar gum, xanthan gum and sodium alginate, at an amount sufficient to stabilize the composition compared to the absence of the emulsion stabilizer, citric acid, at an amount sufficient to change the pH compared to the composition without the citric acid, water, and potassium sorbate and sodium benzoate, at a combined amount sufficient to preserve the composition compared to the absence of the potassium sorbate and sodium benzoate.

In another embodiment, provided are compositions containing 2-amino-4-methylhexane HCl having vasoconstrictor activity, taste-modifying agents including sucralose and a peach flavoring agent, caffeine anhydrous, conjugated linoleic acid, Vitamin B12, chromium picolinate, phosphatidylcholine, a TPGS surfactant, an emulsion stabilizer containing guar gum, xanthan gum and sodium alginate, at an amount sufficient to stabilize the composition compared to the absence of the emulsion stabilizer, citric acid, at an amount sufficient to change the pH compared to the composition without the citric acid, water, and potassium sorbate and sodium benzoate, at a combined amount sufficient to preserve the composition compared to the absence of the potassium sorbate and sodium benzoate.

In one aspect of these embodiments the 2-amino-4-methylhexane is in the composition at a concentration of between at or about 30 mM and at or about 200 mM, the peach flavoring agent is in the composition at an amount between at or about 0.45% and at or about 1.5%, by weight, the sucralose is in the composition in an amount of at or about 1.44%, by weight, the caffeine anhydrous is in the composition in an amount of at or about 2%, by weight, the conjugated linoleic acid (CLA) is in the composition in an amount of at or about 0.25%, by weight, the phosphatidylcholine is in the composition in an amount of at or about 0.075%, by weight, the TPGS surfactant is in the composition in an amount between at or about 0.63% and at or about 1.25%, by weight, the emulsion stabilizer is in the composition at an amount of at or about 0.165%, by weight, the citric acid is in the composition in an amount of at or about 2.66%, by weight, the water is in the composition in an amount between at or about 89.5% and at or about 90.4%, by weight, the potassium sorbate is in the composition in an amount of at or about 0.04%, by weight; and the sodium benzoate is in the composition in an amount of at or about 0.04%, by weight, of the composition.

In another aspect, the 2-amino-4-methylhexane is in the composition at a concentration of at or about 107 mM, the peach flavoring agent is in the composition at an amount at or about 0.663%, by weight, of the composition, the sucralose is in the composition in an amount of at or about 1.438%, by weight, the caffeine anhydrous is in the composition in an amount of at or about 2%, by weight, the conjugated linoleic acid (CLA) is in the composition at an amount of at or about 0.248%, by weight, the Vitamin B12 is in the composition in an amount of at or about 0.025%, by weight, the chromium picolinate is in the composition in an amount of at or about 0.015%, by weight, the phosphatidylcholine is in the composition in an amount of at or about 0.075%, by weight, the TPGS surfactant is in the composition in an amount of at or about 0.625%, by weight, the emulsion stabilizer is in the composition in an amount of at or about 0.165%, by weight, the citric acid is in the composition in an amount of at or about 2.66%, by weight, of the composition, the water is in the composition in an amount of at or about 90.388%, by weight, the potassium sorbate is in the composition in an amount of at or about 0.04%, by weight, and the sodium benzoate is in the composition in an amount of at or about 0.04%, by weight.

In another embodiment, provided are compositions containing 2-amino-4-methylhexane HCl, taste-modifying agents including sucralose and a mint flavoring agent, caffeine anhydrous, conjugated linoleic acid, phosphatidylcholine, a TPGS surfactant, an emulsion stabilizer containing guar gum, xanthan gum and sodium alginate, at an amount sufficient to stabilize the composition compared to the absence of the emulsion stabilizer, citric acid, at an amount sufficient to change the pH compared to the composition without the citric acid, water, and potassium sorbate and sodium benzoate, at a combined amount sufficient to preserve the composition compared to the absence of the potassium sorbate and sodium benzoate.

In one aspect of this embodiment, the 2-amino-4-methylhexane is in the composition at a concentration of at or about 107 mM, the mint flavoring agent is in the composition at an amount of at or about 1.09%, by weight, the sucralose is in the composition in an amount of at or about 1.44%, by weight, the caffeine anhydrous is in the composition in an amount of at or about 2%, by weight, the conjugated linoleic acid (CLA) is in the composition in an amount of at or about 0.25%, by weight, the phosphatidylcholine is in the composition in an amount of at or about 0.075%, by weight, the TPGS surfactant is in the composition in an amount of at or about 1.25%, by weight, the emulsion stabilizer is in the composition in an amount of at or about 0.165%, by weight, the citric acid is in the composition in an amount of at or about 0.17%, by weight, the water is in the composition in an amount at or about 91.9%, by weight, the potassium sorbate is in the composition in an amount of at or about 0.04%, by weight, and the sodium benzoate is in the composition in an amount of at or about 0.04%, by weight.

In another embodiment, provided are compositions containing 2-amino-4-methylhexane HCl, taste-modifying agents including sucralose and a spearmint flavoring agent, caffeine anhydrous, conjugated linoleic acid, phosphatidylcholine, a TPGS surfactant, an emulsion stabilizer containing guar gum, xanthan gum and sodium alginate, at an amount sufficient to stabilize the composition compared to the absence of the emulsion stabilizer, citric acid, at an amount sufficient to change the pH compared to the composition without the citric acid, water, and potassium sorbate and sodium benzoate, at a combined amount sufficient to preserve the composition compared to the absence of the potassium sorbate and sodium benzoate.

In one aspect, the 2-amino-4-methylhexane is in the composition at a concentration of at or about 107 mM, the spearmint flavoring agent is in the composition at an amount of at or about 1.5%, by weight, the sucralose is in the composition in an amount of at or about 1.44%, by weight, the caffeine anhydrous is in the composition in an amount of at or about 2%, by weight, the conjugated linoleic acid (CLA) is in the composition in an amount of at or about 0.25%, by weight, the phosphatidylcholine is in the composition in an amount of at or about 0.075%, by weight, the TPGS surfactant is in the composition in an amount of at or about 0.75%, by weight, the emulsion stabilizer is in the composition in an amount of at or about 0.165%, by weight, the citric acid is in the composition in an amount of at or about 2.66%, by weight, the water is in the composition in an amount at or about 89.5%, by weight, the potassium sorbate is in the composition in an amount of at or about 0.04%, by weight, and the sodium benzoate is in the composition in an amount of at or about 0.04%, by weight.

In another embodiment, provided are compositions containing 2-amino-4-methylhexane HCl, taste-modifying agents including sucralose and a sour apple flavoring agent, caffeine anhydrous, conjugated linoleic acid, phosphatidylcholine, a TPGS surfactant, an emulsion stabilizer containing guar gum, xanthan gum and sodium alginate, at an amount sufficient to stabilize the composition compared to the absence of the emulsion stabilizer, citric acid, at an amount sufficient to change the pH compared to the composition without the citric acid, water, and potassium sorbate and sodium benzoate, at a combined amount sufficient to preserve the composition compared to the absence of the potassium sorbate and sodium benzoate.

In one aspect, the 2-amino-4-methylhexane is in the composition at a concentration of at or about 107 mM, the sour apple flavoring agent is in the composition at an amount of at or about 0.45%, by weight, the sucralose is in the composition in an amount of at or about 1.44%, by weight, the caffeine anhydrous is in the composition in an amount of at or about 2%, by weight, the conjugated linoleic acid (CLA) is in the composition in an amount of at or about 0.25%, by weight, the phosphatidylcholine is in the composition in an amount of at or about 0.075%, by weight, the TPGS surfactant is in the composition in an amount of at or about 0.75%, by weight, the emulsion stabilizer is in the composition in an amount of at or about 0.165%, by weight, the citric acid is in the composition in an amount of at or about 2.66%, by weight, the water is in the composition in an amount at or about 90.515%, by weight, the potassium sorbate is in the composition in an amount of at or about 0.04%, by weight, and the sodium benzoate is in the composition in an amount of at or about 0.04%, by weight.

In another embodiment, provided are compositions containing 2-amino-4-methylhexane HCl, taste-modifying agents including sucralose and a peppermint or cinnamon flavoring agent, caffeine anhydrous, conjugated linoleic acid, phosphatidylcholine, a TPGS surfactant, an emulsion stabilizer containing guar gum, xanthan gum and sodium alginate, at an amount sufficient to stabilize the composition compared to the absence of the emulsion stabilizer, citric acid, at an amount sufficient to change the pH compared to the composition without the citric acid, water, and potassium sorbate and sodium benzoate, at a combined amount sufficient to preserve the composition compared to the absence of the potassium sorbate and sodium benzoate.

In one aspect, the 2-amino-4-methylhexane is in the composition at a concentration of at or about 107 mM, the peppermint or cinnamon flavoring agent is in the composition at an amount of at or about 1%, by weight, the sucralose is in the composition in an amount of at or about 1.44%, by weight, the caffeine anhydrous is in the composition in an amount of at or about 2%, by weight, the conjugated linoleic acid (CLA) is in the composition in an amount of at or about 0.25%, by weight, the phosphatidylcholine is in the composition in an amount of at or about 0.075%, by weight, the TPGS surfactant is in the composition in an amount of at or about 0.75%, by weight, the emulsion stabilizer is in the composition in an amount of at or about 0.165%, by weight, the citric acid is in the composition in an amount of at or about 2.66%, by weight, the water is in the composition in an amount at or about 90%, by weight, the potassium sorbate is in the composition in an amount of at or about 0.04%, by weight and the sodium benzoate is in the composition in an amount of at or about 0.04%, by weight.

In another embodiment, provided are compositions containing 2-amino-4-methylhexane HCl, taste-modifying agents including sucralose and a mint flavoring agent, caffeine anhydrous, L-taurine, alpha lipoic acid, conjugated linoleic acid, phosphatidylcholine, a TPGS surfactant, an emulsion stabilizer containing guar gum, xanthan gum and sodium alginate, at an amount sufficient to stabilize the composition compared to the absence of the emulsion stabilizer, citric acid, at an amount sufficient to change the pH compared to the composition without the citric acid, water, and potassium sorbate and sodium benzoate, at a combined amount sufficient to preserve the composition compared to the absence of the potassium sorbate and sodium benzoate.

In one aspect of this embodiment, the 2-amino-4-methylhexane is in the composition at a concentration of at or about 107 mM, the mint flavoring agent is in the composition at an amount of at or about 1.09%, by weight, the sucralose is in the composition in an amount of at or about 1.44%, by weight, the caffeine anhydrous is in the composition in an amount of at or about 2%, by weight, the L-taurine is in the composition in an amount of at or about 0.25%, by weight, the alpha lipoic acid is in the composition in an amount of at or about 0.025%, by weight, the conjugated linoleic acid (CLA) is in the composition in an amount of at or about 0.25%, by weight, the phosphatidylcholine is in the composition in an amount of at or about 0.075%, by weight, the TPGS surfactant is in the composition in an amount of at or about 1.5%, by weight, the emulsion stabilizer is in the composition in an amount of at or about 0.165%, by weight, the citric acid is in the composition in an amount of at or about 0.17%, by weight, the water is in the composition in an amount at or about 91.3%, by weight, the potassium sorbate is in the composition in an amount of at or about 0.04%, by weight, and the sodium benzoate is in the composition in an amount of at or about 0.04%, by weight.

Also provided are articles of manufacture containing the compositions, such as for administration of the compositions to a subject. For example, the compositions can be packaged in a container. Exemplary of the containers include, but are not limited to, ampoules, such as twist-top ampoules, vials, bags, tubes, bottles and syringes. Exemplary of the containers are containers, e.g. ampoules, containing soft plastic, such as, but not limited to low-density polyethylene (LDPE), for example, ampoules having a capacity of at or about 10, 9, 8, 7, 6, 5, 4, 4.5, 3, 2 or 1 mL, to contain the low-volume compositions.

Typically, the provided compositions have a pH between at or about 2.0 and at or about 5, such as at or about 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4 or 5.

Also provided are methods for producing the compositions, e.g. the palatable liquid compositions containing the aminoalkanes or biocompatible derivatives thereof. In practicing the methods, an oil phase is generated by adding oil phase ingredients to an oil phase vessel, e.g. a tank, a water phase is generated by adding water phase ingredients to a water phase vessel, and the water and oil phases are combined, typically forming an emulsion, followed by the addition of other ingredients, such as taste-modifying agents and pH adjusting agents, and optionally, analysis of the resulting compositions. The oil and water phases can be produces simultaneously, or sequentially, in any order. The methods can be used to produce any of the provided compositions. Producing the oil phase can be carried out by mixing the oil phase ingredients (e.g., with a standard mixer or homogenizer), and/or heating the oil phase ingredients, for example, to at or about 60° C., for example, using a heating apparatus such as a water jacket. Producing the water phase can be carried out by mixing the water phase ingredients (e.g., with a standard mixer or homogenizer), and/or heating the water phase ingredients, for example, to at or about 60° C., for example, using a heating apparatus such as a water jacket. Combining the oil and water phases typically is carried out using a homogenizer and can be carried out while cooling, e.g. rapidly cooling the mixture, e.g. to between at or about 30° C. and 35° C., such as by passing through a recirculating cooler.

Oil phase ingredients can include, but are not limited to, surfactants, active ingredient, e.g. nutritional supplements, vitamins, minerals, fatty acids, and weight-loss compounds, such as, but not limited to, oils, e.g. fatty acids, such as conjugated linoleic acid (CLA), emulsion stabilizers, co-surfactants, and pH adjusting agents.

The water phase ingredients can include, but are not limited to, water, the aminoalkane or aminoalkane derivative, preservatives, e.g. sodium benzoate and potassium sorbate, active ingredient, e.g. nutritional supplements, vitamins, minerals, fatty acids, and weight-loss compounds, such as water soluble vitamins, such as, but not limited to Vitamin B12, chromium picolinate, and caffeine, emulsion stabilizers, surfactants, co-surfactants, and pH adjusting agents.

The methods can include adjusting the pH of the composition, for example, such that the pH is between at or about 2 and 5, such as at or about 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4 or 5, by adding a pH adjusting agent and analyzing the pH of the composition.

The methods further include packaging the compositions into the containers, e.g. into the ampoules, vials, bags, tubes, bottles or syringes, including the twist-top ampoule, such as containers made of a soft plastic, such as, but not limited to low-density polyethylene (LDPE).

Also provided are methods for providing an aminoalkane or biocompatible derivative thereof to a subject by administering the compositions, including any of the compositions provided herein. Typically, the administration is oral. In one example, when the composition is contained in an ampoule, the administration is carried out by breaking a seal on the ampoule, such as by twisting a twist-top on the ampoule.

### DETAILED DESCRIPTION

### Outline

A. DEFINITIONS

B. COMPOSITIONS CONTAINING AMINOALKANES AND DERIVATIVES THEREOF
1. Active ingredients
   a. Aminoalkanes and derivatives thereof
      (i) Vasoconstrictor activity
      (ii) Amounts and activity of the aminoalkane active ingredients
   b. Other active ingredients
2. Taste-modifying agents
3. Surfactants and co-surfactants
   a. TPGS surfactants
   b. Co-surfactants
4. Water
5. Preservatives and Sterilizers
6. pH adjusting agents
7. Emulsion stabilizers
8. Volume of the compositions
9. Palatability of the compositions
10. Exemplary compositions

C. METHODS FOR PRODUCING THE COMPOSITIONS
1. Equipment for use in the methods
   a. Scales
   b. Purifiers
   c. Vessels
   d. Mixers
   e. Heating/Cooling apparatuses
   f. Transfer means
   g. Evaluation equipment
2. General methods for producing the compositions
   a. Water phase ingredients
   b. Water phase production
   c. Oil phase ingredients
   d. Oil phase production
   e. Combining phases
   f. Cooling
   g. Filtration, additions, evaluation and packaging
   h. Cleaning the equipment
3. Analysis of Vasoconstrictor activity

D. COMBINATIONS, ARTICLES OF MANUFACTURE AND KITS
Ampoules

E. METHODS FOR PROVIDING THE ACTIVE INGREDIENTS BY DELIVERY OF THE COMPOSITIONS

F. EXAMPLES

### A. DEFINITIONS

Unless defined otherwise, all technical and scientific terms used herein have the same meaning as is commonly understood by one of skill in the art to which the invention(s) belong. All patents, patent applications, published applications and publications, Genbank sequences, databases, websites and other published materials referred to throughout the entire disclosure herein, unless noted otherwise, are incorporated by reference in their entirety. In the event that there are a plurality of definitions for terms herein, those in this section prevail. Where reference is made to a URL or other such identifier or address, it understood that such identifiers can change and particular information on the internet can come and go, but equivalent information can be found by searching the internet. Reference thereto evidences the availability and public dissemination of such information.

As used herein, aminoalkanes are compounds whose structure is set forth in Formula I:

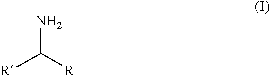

where R is an alkyl and R′ is a hydrogen or an alkyl, where the alkyls are straight chain or branched and substituted or unsubstituted alkyls. Typically, the aminoalkanes are compounds having the structure of Formula I, where R is C₁-C₂₀ alkyl, typically C₂-C₈ alkyl, and R′ is hydrogen or C₁-C₂₀ alkyl, typically C₁-C₈ alkyl.

As used herein, C₁-Cₓ includes C₁-C₂, C₁-C₃ . . . C₁-Cₓ.

As used herein, the term “alkyl” refers to straight or branched chain substituted or unsubstituted hydrocarbon groups having any number of carbon atoms; number of carbon atoms can be specified, for example, 1 to 20 carbon atoms, 1 to 10 carbon atoms, 2 to 9 carbon atoms and 6 to 9 carbon atoms. An alkyl group can be a “saturated alkyl,” meaning that it does not contain any alkene or alkyne groups; alkyl groups optionally can be substituted. An alkyl group can be an “unsaturated alkyl,” meaning that it contains at least one alkene or alkyne group. An alkyl group that includes at least one carbon-carbon double bond (C═C) also is referred to by the term “alkenyl,” and alkenyl groups optionally can be substituted. An alkyl group that includes at least one carbon-carbon triple bond (C≡C) also is referred to by the term “alkynyl,” and alkynyl groups optionally can be substituted.

As used herein, 2-aminoalkanes are aminoalkanes having an —NH₂ group on the second carbon of the carbon chain; 2-aminoalkanes have structures set forth in Formula II:

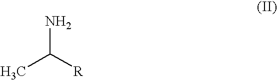

where R is an alkyl as described above; typically, the 2-aminoalkanes used herein have a structure set forth in Formula II, where R is selected from among C₁-C₂₀ alkyl, and typically having between 2 and 9, typically between 4 and 9, carbon atoms. The 2-aminoalkanes can further contain another NH₂ group, for example, on another carbon of the carbon chain, such as on the third or fourth carbon. Exemplary of 2-aminoalkanes are 2-aminoalkanes containing one or more methyl groups, such as, but not limited to, 2-amino-4-methylhexane (also known as 1,3-dimethylamylamine, 1,3-dimethylpentylamine, 2-amino-4-methylhexane, 4-methyl-2-hexylamine, methylhexaneamine, AMH, Forthane and 4-methyl-2-hexanamine), 2-aminoheptane, 2-amino-4-methylheptane, 3-methylhexan-2-amine, 5-methylhexan-2-amine, 3-ethylhexan-2-amine, and 4-methylhexane-2,5-diamine.

As used herein, 2-amino-4-methylhexane refers to a compound of formula I above (where R is 2-methylbutane and R′ is methyl), having the structure:

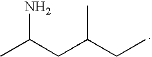

Whenever it appears herein, a numerical range such as “1 to 20” refers to each integer in the given range; for example, “1 to 20 carbon atoms” means that an alkyl group can contain only 1 carbon atom, 2 carbon atoms, 3 carbon atoms, etc., up to and including 20 carbon atoms, although the term “alkyl” also includes instances where no numerical range of carbon atoms is designated. To refer to the number of carbon atoms, an alkyl can be designated as “C₁-C₄ alkyl” or by similar designations. By way of example only, “C₁-C₄ alkyl” indicates an alkyl having one, two, three, or four carbon atoms, i.e., the alkyl is selected from among methyl, ethyl, propyl, iso-propyl, n-butyl, iso-butyl, sec-butyl and t-butyl. Thus “C₁-C₄” includes C₁-C₂, C₁-C₃, C₂-C₃ and C₂-C₄ alkyl. Alkyls include, but are not limited to, methyl, ethyl, propyl, isopropyl, butyl, isobutyl, tertiary butyl, pentyl, hexyl, ethenyl, propenyl, butenyl, hexenyl, ethynyl, propynyl, butynyl and hexynyl.

As used herein, the term “aromatic” refers to a planar ring having a delocalized π-electron system containing 4 n+2 π electrons, where n is an integer. Aromatic rings can be formed by five, six, seven, eight, nine, or more than nine atoms. Aromatics can be optionally substituted. Examples of aromatic groups containing substitutions include, but are not limited to, phenyl, 3-halophenyl, 4-halophenyl, 3-hydroxyphenyl, 4-hydroxy-phenyl, 3-aminophenyl, 4-aminophenyl, 3-methylphenyl, 4-methylphenyl, 3-methoxyphenyl, 4-methoxyphenyl, 4-trifluoromethoxyphenyl, 3-cyano-phenyl, 4-cyanophenyl, dimethylphenyl, hydroxymethyl-phenyl and (trifluoromethyl)phenyl.

As used herein, a subject includes an animal, including a mammal, such as a human.

As used herein, colloid refers to a mixture containing two phases, a dispersed phase and a continuous phase, the dispersed phase containing particles (droplets) distributed throughout the continuous phase. Colloidal mixtures include aerosols, foams and dispersions, for example, emulsions. A liquid colloid, for example, an emulsion, can have a similar appearance, for example, clarity, to a solution, in which there is no dispersed phase.

As used herein, emulsion refers to a colloidal dispersion of two immiscible liquids, for example, an oil and water (or other aqueous liquid), one of which is part of a continuous phase and the other of which is part of a dispersed phase. The provided compositions include emulsions, typically oil-in-water emulsions, in which the oil phase is the dispersed phase and the water phase is the continuous phase. Emulsions typically are stabilized by one or more surfactants and/or co-surfactants and/or emulsion stabilizers. Surfactants form an interfacial film between the oil and water phase of the emulsion, providing stability.

As used herein, “surfactant” and “surface active agent” refer synonymously to synthetic and naturally occurring amphiphilic molecules, for example, molecules having hydrophobic portion(s) and hydrophilic portion(s). Due to their amphiphilic (amphipathic) nature, surfactants and co-surfactants typically can reduce the surface tension between two immiscible liquids, for example, the oil and water phases in an emulsion, stabilizing the emulsion. Different surfactants can be characterized based on their relative hydrophobicity and/or hydrophilicity. For example, relatively lipophilic surfactants are more soluble in fats, oils and waxes, typically having HLB values less than 10 or about 10, while relatively hydrophilic surfactants are more soluble in aqueous compositions, for example, water, and typically have HLB values greater than 10 or about 10. Relatively amphiphilic surfactants are soluble in oil and water based liquids and typically have HLB values close to 10 or about 10.

Surfactants include, for example, soaps, detergents, lipids, emulsifiers, dispersing agents and wetting agents, molecules that emulsify liquids, for example, by forming an emulsion in an aqueous medium or aqueous liquid dilution composition, for example, forming a colloidal dispersion of two immiscible liquids in the form of droplets, for example, an emulsion such as a microemulsion; and compounds that form various macromolecular structures, for example, aggregates, in liquids, for example, micelles, lipid bilayer structures, including liposomes, and inverse micelles.

As used herein, a PEG derivative of Vitamin E is a compound containing one or more Vitamin E moieties (e.g. a tocopherol or tocotrienol) joined, for example by an ester, ether amide or thioester bond, with one or more polyethylene glycol (PEG) moieties, via a linker, for example a dicarboxylic or tricarboxylic acid. Exemplary of PEG derivatives of Vitamin E are tocopherol polyethylene glycol succinate (TPGS), TPGS analogs, TPGS homologs and TPGS derivatives.

As used herein, a tocopherol polyethylene glycol diester (TPGD) is a PEG-derivative of tocopherol where the linker is a dicarboxylic acid (a carboxylic acid having two carboxy groups, e.g. succinic acid), such as succinic acid. Exemplary of dicarboxylic acids that can be used as linkers in these tocopherol and tocotrienol PEG diester surfactants are succinic acid, sebacic acid, dodecanedioic acid, suberic acid, or azelaic acid, citraconic acid, methylcitraconic acid, itaconic acid, maleic acid, glutaric acid, glutaconic acid, fumaric acids and phthalic acids. Exemplary of TPGDs are tocopherol succinate polyethylene glycol (TPGS), tocopherol sebacate polyethylene glycol, tocopherol dodecanedioate polyethylene glycol, tocopherol suberate polyethylene glycol, tocopherol azelaate polyethylene glycol, tocopherol citraconate polyethylene glycol, tocopherol methylcitraconate polyethylene glycol, tocopherol itaconate polyethylene glycol, tocopherol maleate polyethylene glycol, tocopherol glutarate polyethylene glycol, tocopherol glutaconate polyethylene glycol, and tocopherol phthalate polyethylene glycol, among others.

As used herein, “tocopherol polyethylene glycol succinate” “TPGS,” “tocopheryl polyethylene glycol succinate surfactant” and “TPGS surfactant” refer to tocopherol polyethylene glycol (PEG) diesters, that are formed by joining, via esterification, tocopherol succinate, which itself is an ester made by esterification of tocopherol and succinic acid. The PEG moiety of the TPGS surfactant can be any PEG moiety, for example, PEG moieties between 200 or about 200 and 20,000 or about 20,000 KDa, typically between 200 or about 200 and 6000 or about 6000 KDa, for example, between 600 or about 600 KDa and 6000 or about 6000 KDa, typically between 200 or about 200 KDa and 2000 or about 2000 KDa, between 600 or about 600 KDa and 1500 or about 1500 KDa, 200 or about 200 KDa, 300 or about 300 KDa, 400 or about 400 KDa, 500 or about 500 KDa, 600 or about 600 KDa, 800 or about 800 KDa, and 1000 or about 1000 KDa, and PEG moieties that are modified, for example, methylated PEG (m-PEG) and/or PEG moieties including other PEG analogs, e.g. PEG-NHS, PEG-aldehyde, PEG-SH, PEG-NH₂, PEG-CO₂H, and branched PEGs.

Exemplary of the TPGS surfactants is TPGS-1000, which has a PEG moiety of 1000 KDa. The TPGS can be any natural, water-soluble, tocopherol polyethylene glycol succinate, for example, the food grade TPGS sold under the name Eastman Vitamin E TPGS®, food grade, by Eastman Chemical Company, Kingsport, Tenn. This TPGS is water-soluble form of natural-source vitamin E, which is prepared by esterifying the carboxyl group of crystalline d-alpha-tocopheryl acid succinate with polyethylene glycol 1000 (PEG 1000), and contains between 260 and 300 mg/g total tocopherol. A similar compound can be made by esterifying the carboxyl group of the d,1 form of synthetic Vitamin E with PEG 1000. It forms a clear liquid when dissolved 20% in water. This tocopheryl polyethylene glycol is a water-soluble preparation of a fat-soluble vitamin (vitamin E), for example, as disclosed in U.S. Pat. Nos. 3,102,078 and 2,680,749 and U.S. Published Application Nos. 2007/0184117 and 2007/0141203. Also exemplary of the TPGS surfactant that can be used in the provided compositions is the Water Soluble Natural Vitamin E (TPGS), sold by ZMC-USA, The Woodlands, Tex. Any known source of TPGS can be used. Typically, the TPGS surfactant is GRAS and Kosher certified. TPGS typically has an HLB value of between 16 or about 16 and 18 or about 18.

As used herein, analog refers to a chemical compound that is structurally similar to another compound (referred to as a parent compound), but differs slightly in composition, for example, by the variation, addition or removal of an atom, one or more units (e.g. methylene unit(s)-(CH₂)ₙ) or one or more functional groups. The analog can have different chemical or physical properties compared with the original compound and/or can have improved biological and/or chemical activity. Alternatively, the analog can have similar or identical chemical or physical properties compared with the original compound and/or can have similar or identical biologic and/or chemical activity For example, the analog can be more hydrophilic or it can have altered reactivity as compared to the parent compound. The analog can mimic the chemical and/or biologically activity of the parent compound (i.e., it can have similar or identical activity), or, in some cases, can have increased or decreased activity. The analog can be a naturally or non-naturally occurring (e.g. synthetic) variant of the original compound. Other types of analogs include isomers (enantiomers, diasteromers, and the like) and other types of chiral variants of a compound, as well as structural isomers. The analog can be a branched or cyclic variant of a linear compound. For example, a linear compound can have an analog that is branched or otherwise substituted to impart certain desirable properties (e.g., improve hydrophilicity or bioavailability). Exemplary of the analogs used in the provided compositions and methods are TPGS analogs, which typically are used as surfactants, for example, in place of the TPGS parent compound in any of the provided compositions.

As used herein, homolog refers to an analog that differs from the parent compound only by the presence or absence of a simple unit, such as a methylene unit, or some multiple of such units, e.g., —(CH₂)ₙ—. Typically, a homolog has similar chemical and physical properties as the parent compound. Exemplary of the homologs used in the provided compositions and methods are TPGS homologs.

As used herein, HLB refers to a value that is used to index and describe a surfactant according to its relative hydrophobicity/hydrophilicity, relative to other surfactants. A surfactant's HLB value is an indication of the molecular balance of the hydrophilic and lipophilic portions of the surfactant, which is an amphipathic molecule. Each surfactant and mixture of surfactants (and/or co-surfactants) has an HLB value that is a numerical representation of the relative weight percent of hydrophobic and hydrophilic portions of the surfactant molecule(s). HLB values are derived from a semi-empirical formula. The relative weight percentages of the hydrophobic and hydrophilic groups are indicative of surfactant properties, including the molecular structure, for example, the types of aggregates the surfactant will form and the solubility of the surfactant. See, for example, Griffin, W. C. J. Soc. Cos. Chem. 1:311 (1949).

Surfactant HLB values range from 1-45, while the range for non-ionic surfactants typically is from 1-20. The more lipophilic a surfactant is, the lower its HLB value. Conversely, the more hydrophilic a surfactant is, the higher its HLB value. Lipophillic surfactants have greater solubility in oil and lipophilic substances, while hydrophilic surfactants dissolve more easily in aqueous media. In general, surfactants with HLB values greater than 10 or greater than about 10 are called “hydrophilic surfactants,” while surfactants having HLB values less than 10 or less than about 10 are referred to as “hydrophobic surfactants.” HLB values have been determined and are available for a plurality of surfactants (e.g. see U.S. Pat. No. 6,267,985). It should be appreciated that HLB values for a given surfactant or co-surfactant can vary, depending upon the empirical method used to determine the value. Thus, HLB values of surfactants and co-surfactants provide a rough guide for formulating compositions based on relative hydrophobicity/hydrophilicity. For example, a surfactant typically is selected from among surfactants having HLB values within a particular range of the surfactant or co-surfactant, that can be used to guide formulations. Table 1 lists HLB values of exemplary surfactants and co-surfactants.

TABLE 1
HLB Values of Exemplary Surfactants and Co-Surfactants

| Surfactant/ | | Surfactant/ | |
|---|---|---|---|
| co-surfactant | HLB | co-surfactant | HLB |
| PEG-2 Hydrogenated | 1.7 | PEG-10 oleyl ether | 12.4 |
| Castor Oil | | | |
| Sorbitan Trioleate | 1.8 | PEG-8 isooctylphenyl ether | 12.4 |
| Sorbitan Tristearate | 2.1 | PEG-10 stearyl ether | 12.4 |
| Glyceryl Stearate | 3.5 | PEG-35 Castor Oil | 12.5 |
| Sorbitan Sesquioleate | 3.7 | PEG-10 cetyl ether | 12.9 |
| Labrafil | 4 | Nonoxynol-9 | 12.9 |
| Sorbitan Oleate | 4.3 | PEG-40 Castor Oil | 13 |
| Sorbitan monostearate | 4.7 | PEG-10 isooctylphenyl ether | 13.5 |
| PEG-2 oleyl ether | 4.9 | PEG-40 Hydrogenated | 14 |
| | | Castor Oil | |
| PEG-2 stearyl ether | 4.9 | Labrasol | 14 |
| PEG-7 Hydrogenated | 5 | Nonoxynol-15 | 14.2 |
| Castor Oil | | | |
| PEG-2 cetyl ether | 5.3 | PEG-12 tridecyl ether | 14.5 |
| PEG-4 Sorbitan Stearate | 5.5 | PEG-18 tridecyl ether | 14.5 |
| PEG-2 Sorbitan | 6 | Polysorbate 60 | 14.9 |
| Isostearate | | | |
| Sorbitan Palmitate | 6.7 | Polysorbate 80 | 15 |
| Triton SP-135 | 8 | PEG-20 Glyceryl Stearate | 15 |
| Sorbitan monolaurate | 8.6 | PEG-20 Stearate | 15 |
| PEG-40 Sorbitan | 9.5 | PEG-20 stearyl ether | 15.3 |
| Peroleate | | | |
| PEG-4 lauryl ether | 9.7 | PEG-20 oleyl ether | 15.3 |
| Polysorbate 81 | 10 | Polysorbate 40 | 15.6 |
| PEG-40 Sorbitan | 10 | PEG20 cetyl ether | 15.7 |
| Hexaoleate | | | |
| PEG-40 Sorbitan | 10 | PEG(20) hexadecyl ether | 15.7 |
| Perisostearate | | | |
| PEG-10 Olive | 10 | PEG-60 Hydrogenated | 16 |
| Glycerides | | Castor Oil | |
| PEG sorbitol hexaoleate | 10.2 | PEG-30 Stearate | 16.5 |
| Polysorbate 65 | 10.5 | Polysorbate 20 | 16.7 |
| PEG-25 Hydrogenated | 10.8 | PEG-75 Lanolin | 16.7 |
| Castor Oil | | | |
| Polysorbate 85 | 11 | PEG23 lauryl ether | 16.9 |
| PEG-7 Glyceryl | 11 | PEG-40 Stearate | 17.3 |
| Cocoate | | | |
| PEG-8 Stearate | 11.1 | PEG-50 Stearate | 17.7 |
| PEG sorbitan tetraoleate | 11.4 | PEG40 isooctylphenyl ether | 17.9 |
| PEG-15 Glyceryl | 12 | PEG-100 Stearate | 18.8 |
| Isostearate | | | |
| PEG-35 Almond | 12 | Pluronic F68 | 29 |
| Glycerides | | | |
| Tocopherol | 16-18 | Phosphatidylcholine | 7.6 |
| polyethylene glycol | | | |
| succinate (TPGS) | | | |

The surfactants and HLB values set forth in Table 1 are exemplary. Any known surfactant or co-surfactant can be used with the provided compositions (e.g. see U.S. Pat. No. 6,267,985). The surfactant(s) contained in the provided compositions typically have an HLB value between 14 or about 14 and 20 or about 20, for example, 14, 15, 16, 17, 18, 19, 20, about 14, about 15, about 16, about 17, about 18, about 19 or about 20. Exemplary of a surfactant that can be used in the provided compositions is a PEG-derivative of Vitamin E, such as tocotrienol or tocopherol PEG diesters, such as TPGS (e.g. TPGS 1000) and TPGS analogs. Other known surfactants having HLB values between 14 or about 14 and 20 or about 20, typically between about 16 and 18, also can be suitable. For example, surfactants having similar properties to TPGS also can be used. Typically, the surfactant is a natural surfactant, for example, a surfactant that is G.R.A.S. (generally recognized as safe) by the FDA and/or Kosher certified.

As used herein, “co-surfactant” is used to refer to a surfactant, typically a phospholipid, that is included in a composition in combination with a surfactant (primary surfactant), to improve the emulsification and/or stability of the composition, for example, to emulsify the ingredients of the composition. Co-surfactants include lipids, e.g. phospholipids, such as, but not limited to, phosphatidylcholine.

As used herein, a phospholipid is an amphipathic, phosphate-containing lipid, for example, a molecule containing one phosphate, a glycerol and one or more fatty acids. In one example, one or more phospholipids is used as a co-surfactant in the provided compositions. Exemplary of the phospholipids used in the provided compositions are lecithin, including phosphatidylcholine (PC), phosphatidylethanolamine (PE), distearoylphosphatidylcholine (DSPC), phosphatidylserine (PS), phosphatidylglycerol (PG), phosphatidic acid (PA), phosphatidylinositol (PI), sphingomyelin (SPM) or a combination thereof. Typically, the phospholipid is phosphatidylcholine (PC), which sometimes is referred to by the general name “lecithin.” Exemplary of the phospholipids that can be used as co-surfactants in the provided compositions are the phospholipids sold by Lipoid, LLC, Newark, N.J., for example, Purified Egg Lecithins, Purified Soybean Lecithins, Hydrogenated Egg and Soybean Lecithins, Egg Phospholipids, Soybean Phospholipids, Hydrogenated Egg and Soybean Phospholipids. Synthetic Phospholipids, PEG-ylated Phospholipids and phospholipid blends sold by Lipoid, LLC. Exemplary of the phosphatidylcholine that can be used as a co-surfactant in the provided compositions is the phosphatidylcholine composition sold by Lipoid, LLC, under the name Lipoid S100, which is derived from soy extract and contains greater than 95% or greater than about 95% phosphatidylcholine.

As used herein, additives include anything that one can add to a food, beverage, or other human consumable, to enhance one or more of its nutritional, pharmaceutical, dietary, health, nutraceutical, health benefit, energy-providing, treating, holistic, or other properties, such as the ability to cause promote or improve a biologic effect, such as sympathomimetic effects, stimulatory effects, vasoconstriction, decongestion (e.g. bronchial or nasal decongestion), increased energy, endurance, mood-enhancement, appetite suppression and/or weight loss. Additives include nutraceuticals, pharmaceuticals, vitamins (e.g. Vitamin B12), stimulants (e.g. caffeine and caffeine anhydrous), aminoalkanes, e.g. 2-amino-4-methylhexane and derivatives thereof, such as 2-amino-4-methylhexane HCl, fatty acids, such as essential fatty acids, e.g. polyunsaturated fatty acids, for example, omega-3 fatty acids and omega-6 fatty acids, for example conjugated linoleic acid CLA.

As used herein, “amount” refers to the quantity (e.g. molecular quantity such as number of moles), mass (e.g. grams or milligrams), concentration (e.g. molarity, weight/volume, weight/weight, volume/volume, or other means to quantitatively describe a compound, substance or composition.

As used herein, an effective amount of a compound, such as an additive or an active ingredient (e.g. the aminoalkane active ingredients), refers to the mass, quantity, such as molecular quantity (e.g. moles) and/or concentration of the additive necessary for causing a desired biologic effect, e.g. sympathomimetic effects, stimulatory effects, vasoconstriction, decongestion (e.g. bronchial or nasal decongestion), increased energy, endurance, mood-enhancement, appetite suppression and/or weight loss or preventing, curing, ameliorating, arresting or partially arresting a symptom of a disease or disorder, or the quantity and/or concentration desired by an individual for intake, such as daily intake, and/or nutritional supplementation, for example, an amount sufficient to enhance the nutritional, pharmaceutical, nutraceutical, health or energy property of a food, beverage, or other consumable. In one example, the effective amount of the active ingredient is an amount having an equivalent (the same) vasoconstrictor activity to a specified amount of a known vasoconstrictor, e.g. 2-amino-4-methylhexane HCl.

As used herein, unit dose form refers to physically discrete units suitable for human and animal subjects and packaged individually as is known in the art.

As used herein, weight-loss compounds are compounds that cause, promote or increase weight loss of a subject when administered to the subject.

As used herein, stimulants are compounds that cause, increase or promote alertness and/or awakeness or focus in a subject when administered to the subject; the effect can be temporary.

As used herein, “vasoconstrictor” and “pressor” are used synonymously to refer to compounds that cause vasoconstriction (narrowing of blood vessels) when administered to a subject, such as an animal subject or a human.

As used herein, “equivalent activity” is used to describe a property of two compounds or compositions, where the first compound or composition, or a given amount of the first compound or composition, has the same activity (e.g. activity such as vasoconstrictor activity) as the second compound or composition or a given amount of the second compound or composition; the activity is determined by measuring a particular effect of the compounds, such as a effect after administration of the compound to a subject, such as vasoconstriction. Amounts (e.g. mass, molecular quantity or concentration) of the compounds can be specified. For example, an amount of a first compound (e.g. a mass, or molecular quantity (e.g. moles) or concentration of the first compound) can contain equivalent activity to a given amount (e.g. a mass, or molecular quantity (e.g. moles) or concentration) of a second compound. In some cases, the amounts or concentrations of the first and second compounds with equivalent activity are the same (e.g. 1 gram of the first compound contains the same activity as 1 gram of the second compound); in other cases, the amounts or concentrations are different (e.g. 500 milligrams of the first compound contains equivalent activity to 1 gram of the second compound). For example, compounds used in the compositions are aminoalkanes, and biocompatible derivatives thereof, in an amount that is determined with reference to the vasoconstrictor activity of a specified amount of a 2-amino-4-methylhexane HCl. Methods for determining vasoconstrictor activity are well known and include exemplary methods described herein, such as in section C(3), below.

As used herein, “vasoconstrictor activity” refers to the amount of vasoconstriction effected by administration of the compound or composition, e.g. the amount of vasoconstriction that occurs in a subject following administration of the compound; a particular amount of the compound typically is specified, e.g. the vasoconstrictor activity of a concentration of the compound, of a molarity of the compound, or of the number of moles of the compound. Vasoconstrictor activity can be determined by measuring vasoconstriction in the subject indirectly, e.g. by measuring blood pressure of the subject, using methods well-known in the art, such as exemplary methods described herein, including assays that measure vasoconstrictor activity of vasoconstrictors such as epinephrine or adrenaline, e.g. those assays that have been used to measure the vasoconstrictor activity of aminoalkanes, e.g. 2-aminoalkanes (see Swanson and Chen, Journal of Pharmacology and Experimental Therapeutics, 88(1), 10-13 (1946), citing Swanson et al., Journal of Pharmacology and Experimental Therapeutics, 85(1), 70-73 (1945) and Swanson et al., Journal of Pharmacology and Experimental Therapeutics, 79(4), 329-333 (1943). Such assays include, for example, evaluation of vasoconstriction in animal subjects as described in Elliott, J. Physiol. 44: 374-409 (1912), where a test compound is injected intravenously into a pithed animal subject and blood pressure of the subject is measured, and rhinometric assays, where changes in nasal cavity patency (i.e. degree of nasal cavity clearance or blockage) are evaluated in the presence of a test compound (see e.g., Rinder and Lundberg, Acta Physiol Scand 157: 233-244 (1996); Salem and Clemente, Arch Otolaryngology 96: 524-529 (1972) and McLeod et al., J Pharmacological and Toxicological Meth. 48: 153-159 (2002)). Assays for measuring vasoconstrictor activity are described in section C(3) herein.

As used herein, “oil phase” refers to the portion of a composition having more than one phase, e.g. an emulsion, that contains lipophilic (hydrophobic) and/or amphiphilic (amphipathic) ingredient(s) and generally not hydrophilic ingredients, and is, in general, the lipid soluble portion (phase) of the composition; alternatively, “oil phase” can refer to the mixture of some or all of the lipophilic and/or amphiphilic ingredients of a composition having a lipid soluble and a water soluble phase (e.g. emulsions) that are combined during the methods of making the composition to form the lipid-soluble portion (phase) of the composition, for example, prior to combining with the water phase.

As used herein, oil phase ingredients are components of a composition, e.g. an emulsion, that are added to the oil phase during the methods of making the composition; the oil phase ingredients are lipophilic (hydrophobic) and/or amphiphilic (amphipathic).

As used herein, “water phase” refers to the portion of a composition having more than one phase, such as an emulsion, that contains hydrophilic and/or amphiphilic (amphipathic) ingredient(s) and generally does not contain hydrophobic ingredients, and, in general, is the water-soluble (aqueous) portion of the composition; alternatively, “water phase” can refer to the mixture of some or all of the hydrophilic and/or amphipathic ingredients of a composition having a lipid soluble and a water soluble phase (e.g. emulsions) that are combined during the methods of making the composition, to form the aqueous portion (phase) of the composition, e.g. prior to combining with the water phase.

As used herein, water phase ingredients are components of a composition, e.g. and emulsion, that are added to the water phase during the methods of making the composition; the water phase ingredients are hydrophilic and/or amphipathic.

As used herein, “emulsion phase ingredients” or “flavor phase ingredients” refer to ingredients of compositions having more than one phase (e.g. emulsions) that are added during methods of making the composition after the more than one phase (e.g. oil and water phases) have been combined. Exemplary of such ingredients are flavoring agents, pH adjusting agents and other additives.

As used herein, “stability” of a composition refers to its ability to remain free or substantially free from one or more changes (or changes to a lesser degree or compared to a less stable composition), typically undesirable changes, when stored over a period of time, for example, at least at or about, or more than at or about 1, 2, 3, 4, 5, 6 or more days, 1, 2, 3, 4 or more weeks, 1, 2, 3, 4, 5, 6, 7, 8, 9, 10, 11, 12 or more months, 1, 2, 3, 4 or more years, or for a period of time that is greater than a less stable composition; temperature or other conditions of storage can be specified; for example, the compositions can be stable at room temperature, e.g. at or about 25° C., at refrigerated temperatures, e.g. at or about 4° C., or at frozen temperatures, e.g. at or about −20° C. Such changes can include, for example, oxidation, contamination, phase separation, discoloration, change in smell and/or taste, precipitation of ingredients.

As used herein, stabilize means to increase the stability of a composition.

As used herein, “room temperature” and “ambient temperature” refer to temperature common in one or more enclosed spaces where human beings typically are or reside, e.g. a home or place of business; room temperature can vary, but generally refers to temperatures between at or about 19° C. and at or about 25° C. When a composition is stored at room temperature it is generally understood it is kept at a temperature within this range; precise temperatures can be specified.

As used herein, refrigerated temperature refers to a temperature that is common in a refrigerator, for example, a household or restaurant refrigerator, and is cooler than room temperature but at least a few degrees above the freezing point of water (at or about 0° F. or −19° C. or −20° C.); typically, refrigerated temperatures are between at or about 10° C. and at or about 0° C., for example, 4° C., and can vary. When a composition is stored at room temperature, it is understood it is kept at a temperature within this range; a particular temperature can be specified.

As used herein, frozen temperature refers to a temperature at or about or below the freezing point of water, e.g. temperatures commonly used in household freezer, e.g. at or about 0° F. or −19° C. or −20° C., or colder.

As used herein, the singular forms “a,” “an” and “the” include plural referents unless the context clearly dictates otherwise. Thus, for example, with reference to a composition, containing “an active ingredient”″ includes compounds with one or a plurality of active ingredients.

As used herein, ranges and amounts can be expressed as “about” a particular value or range. About also includes the exact amount. Hence “about 5 grams” means “about 5 grams” and also “5 grams.” It also is understood that ranges expressed herein include whole numbers within the ranges and fractions thereof. For example, a range of between 5 grams and 20 grams includes whole number values such as 5, 6, 7, 8, 9, 10, 11, 12, 13, 14, 15, 16, 17, 18, 19 and 20 grams, and fractions within the range, for example, 5.25, 6, 72, 8.5, 11.95, etc grams.

As used herein, “optional” or “optionally” means that the subsequently described event or circumstance does or does not occur and that the description includes instances where said event or circumstance occurs and instances where it does not. For example, an optionally variant portion means that the portion is variant or non-variant. In another example, an optional ligation step means that the process includes a ligation step or it does not include a ligation step.

As used herein, fatty acid refers to straight-chain hydrocarbon molecules with a carboxyl (COOH) group at one end of the chain.

As used herein, polyunsaturated fatty acid and PUFA are used synonymously to refer to fatty acids that contain more than one carbon-carbon double bond in the carbon chain of the fatty acid. PUFAs, particularly essential fatty acids, are useful as dietary supplements.

As used herein, essential fatty acids are fatty acids (e.g. PUFAs) that mammals, including humans, cannot synthesize using any known chemical pathway. Thus, essential fatty acids must be obtained from diet or by supplementation. Exemplary of essential PUFA fatty acids are omega-3 (ω3; n−3) fatty acids and the omega-6 (ω-6; n−6) fatty acids.

As used herein, omega-3 (ω3; n−3) fatty acids are methylene interrupted polyenes, which have two or more cis double bonds, separated by a single methylene group and in which the first double bond appears at the third carbon from the last (ω) carbon. Exemplary of Omega-3 fatty acids are Alpha-Linolenic acid (α-Linolenic acid; ALA) (18:3ω3) (a short-chain fatty acid); Stearidonic acid (18:4ω3) (a short-chain fatty acid); Eicosapentaenoic acid (EPA) (20:5ω3); Docosahexaenoic acid (DHA) (22:6ω3); Eicosatetraenoic acid (24:4ω3); Docosapentaenoic acid (DPA, Clupanodonic acid) (22:5ω3); 16:3 ω3; 24:5 ω3 and nisinic acid (24:6ω3). Longer chain Omega-3 fatty acids can be synthesized from ALA (the short-chain omega-3 fatty acid).

As used herein, omega-6 (ω-6; n−6) fatty acids are methylene interrupted polyenes, which have two or more cis double bonds, separated by a single methylene group and in which the first double bond appears at the sixth carbon from the last (ω) carbon. Exemplary of Omega-6 fatty acids are Linoleic acid (18:2ω6) (a short-chain fatty acid); Gamma-linolenic acid (GLA) (18:3ω6); Dihomo gamma linolenic acid (DGLA) (20:3ω6); Eicosadienoic acid (20:2ω6); Arachidonic acid (AA) (20:4ω6); Docosadienoic acid (22:2ω6); Adrenic acid (22:4ω6); and Docosapentaenoic acid (22:5ω6).

As used herein, “preservative” and “preservativer” are used synonymously to refer to ingredients that improve stability of compositions, and typically are included in the provided compositions at amounts sufficient to improve stability of the compositions. Exemplary of preservatives are potassium sorbate, sodium benzoate, benzyl alcohol, Benzyl Benzoate, Methyl Paraben, Propyl Paraben, antioxidants, for example, Vitamin E, Vitamin A Palmitate and Beta Carotene. Typically, preservatives in the provided compositions are selected based on their safety for human consumption, e.g. in foods/beverages.

As used herein, “solvent” refers to an ingredient, for example, an oil, that dissolves or improves solubility of a compound (e.g. ingredient) in a mixture of ingredients, e.g. an oil or water phase, during the methods for making a composition.

As used herein, “w/w,” “weight per weight,” “by weight” “% by weight” and “weight percent” are used to express the ratio of the mass of one component of a composition compared to the mass of the entire composition or to another compound. For example, when the amount of a particular ingredient represents 1%, by weight (w/w) of a composition, the mass of that ingredient is 1% of the mass of the entire composition. Similarly, when the amount of an ingredient is 50% (w/w) of the composition, the mass of that ingredient is 50% of the entire mass of the composition.

As used herein “v/v,” “volume per volume,” “percent by volume” and “volume percent” are used to express the ratio of the volume of one component of a composition and the volume of the entire composition or to another compound. For example, when the amount of an ingredient of a composition is 1% by volume, or 1 percent by volume, of the composition, the volume of the ingredient is 1% of the total volume of the composition.

As used herein, “emulsion stabilizer” refers to compounds that stabilize, emulsify, prevent phase separation of, and/or change the viscosity of compositions, such as emulsions. Exemplary of emulsion stabilizers are compositions containing a blend of gums, for example, gums used as emulsifying agents, such as, but not limited to, a blend containing one or more of xanthan gum, guar gum and sodium alginate, for example, the emulsion stabilizer sold under the brand name SALADIZER®, available from TIC Gums, Inc. (Belcamp, Md.). Emulsion stabilizers further include compositions with other gums, for example, gum acacia and sugar beet pectin, and other gums.

As used herein, a pH adjuster is any compound, typically an acid or a base, that changes the pH of a composition, for example, reduces the pH of the composition or increases the pH of the composition, typically without altering other properties of the composition, or without substantially altering other properties. pH adjusters are well known. Exemplary of the pH adjusters are acids, for example, citric acid and phosphoric acid, and bases.

As used herein, “taste-modifying agents” include compounds that modify the flavor or sweeten a composition, or mask a flavor (e.g. unpleasant flavor) in a composition, and generally render the composition palatable or improve palatability of the composition. The taste-modifying agents include but are not limited to, sweetening agents and flavoring agents, including taste/flavor masking agents and agents that confer a flavor to the composition. The provided compositions typically contain a combination of taste-modifying agents including a flavoring agent and a sweetening agent, such as a sugar substitute, e.g. sucralose.

As used herein, a flavoring agent is a compound that changes, typically improves, the taste and/or smell of the provided composition or masks a flavor or taste (typically an unpleasant flavor or taste) in the composition. Exemplary flavoring agents include, but are not limited to, flavoring agents that confer fruit flavors, such as, but not limited to, guava, kiwi, peach, mango, papaya, passion fruit, pineapple, grape, banana, strawberry, raspberry, blueberry, cranberry, orange, grapefruit, tangerine, lemon, lime, cherry, apple, plum, watermelon, coconut, and other fruit flavors; cola flavors, tea flavors, coffee flavors, chocolate flavors, dairy flavors, root beer and birch beer flavors, methyl salicylate (wintergreen oil, sweet birch oil), citrus oils (e.g. lemon oil) essential oils or water soluble extracts of menthol, wintergreen, peppermint, sweet mint, mint, spearmint, natural and artificial vanilla, nut flavor, nuts, chocolate, fudge, butterscotch, cinnamon, clove, rose, spice, violet, herbal, durean, green tea, butter, cream custard, chamomile. Particular examples of flavoring agents include a peach flavoring agent sold by Mission Flavors and Fragrances, Inc., Foothill Ranch, Calif., (Catalog number PH-147), a spearmint flavoring agent sold by Mission Flavors and Fragrances, Inc. (Catalog number MI-110), a peppermint flavoring agent sold by Mission Flavors and Fragrances, Inc. (Catalog number MI-104), a mint flavoring agent sold by Mission Flavors and Fragrances, Inc. (Catalog number L-12389), another mint flavor sold by Mission Flavors and Fragrances (Catalog number MI-119), a cinnamon flavoring agent sold by Mission Flavors and Fragrances, Inc. (Catalog number L-9806) and a sour apple flavoring agent sold by Mission Flavors and Fragrances, Inc. (Catalog number AP-138), which confer peach, spearmint, peppermint, cinnamon and sour apple flavors to compositions, respectively.

As used herein, “sweeteners” and “sweetening agents” refer to compounds that increase the sweetness of a composition. Sweetening agents include, but are not limited to, sugar or sugar substitutes, such as sucralose, dextrose, lactose, mannitol, sucrose, xylitol, malitol, acesulfame potassium, talin, glycyrrhizin, aspartame, saccharin, sodium saccharin, neotame, acesulfame potassium; sodium cyclamate; and honey.

As used herein, “not more than,” “NMT,” and “at least” refer to an amount that is less than or equal to the amount following the phrase. Similarly, “not less than” and “NLT” refer to an amount that is greater than or equal to the amount that follows the phrase.

As used herein, natural is used to refer to compounds and ingredients that can be found in nature and are not solely man-made. Natural ingredients include natural surfactants, such as TPGS surfactants.

As used herein, “G.R.A.S.” and “GRAS” are used synonymously to refer to compounds, compositions and ingredients that are “Generally Regarded as Safe” by the USDA, FDA for use as additives, for example, in foods, beverages and/or other substance for human consumption, for example, any substance that meets the criteria of sections 201(s) and 409 of the U.S. Federal Food, Drug and Cosmetic Act.

As used herein, kosher is used to refer to substances that conform to Jewish Kosher dietary laws, for example, substances that do not contain ingredients derived from non-kosher animals or ingredients that were not made following kosher procedures. Typically, the compositions provided herein are Kosher certified.

As used herein, vessel refers to any container, for example, tanks, pots, vials, flasks, cylinders and beakers, that is used in the provided methods for making the compositions to contain ingredients, mixtures of ingredients, phases of the compositions, and the compositions, for example, used to mix and heat or cool the compositions or phases thereof. Vessels include tanks, such as water phase tanks and oil phase tanks, used to mix the water and oil phases, and packaging and holding tanks, which typically contain the final or nearly final compositions. Tanks include tanks having heating and/or cooling devices, such as water-jacketed tanks and tanks attached to recirculating coolers; and tanks with mixers mounted, e.g. to the top of the tank.

As used herein, a water phase vessel refers to the vessel in which the water phase of a composition is generated (e.g. to which the water phase ingredients are added, mixed and/or heated/cooled). Exemplary of water phase vessels are water phase tanks.

As used herein, an oil phase vessel refers to the vessel in which the oil phase of a composition is generated (e.g. to which the oil phase ingredients are added, mixed and/or heated/cooled). Exemplary of oil phase vessels are oil phase tanks.

As used herein, transfer means refers to any equipment, combination of equipment and/or system that is to transfer a composition (e.g. a liquid composition) or portion thereof, for example, from one vessel to another vessel. Exemplary of the transfer means are a transfer pump and appropriate fittings, for example, sanitary fittings, ball valves and transfer hoses, for example, food grade hoses, and manual transfer means such as pouring or pipetting.

As used herein a mixer is any piece of equipment or combination of equipment that can be used to mix ingredients in the provided methods for making the compositions, for example, standard mixers and homogenizers.

As used herein, standard mixers are mixers that combine a group of ingredients, for example, oil phase ingredients or water phase ingredients, or to mix one or more ingredients into a liquid such as an emulsion. Standard mixers include any mixers that move material, for example, ingredients, for example, to promote dissolving of the ingredients.

As used herein, “homogenizer” refers to mixers that can be used to form an emulsion. The homogenizers typically are capable of high-shear mixing.

As used herein, a cooling apparatus is any piece of equipment or combination of equipment that is used to cool a composition or phase or ingredient thereof, for example, during mixing and/or homogenizing, for example, to chill the mixture while emulsifying the oil and water phases. Exemplary of the cooling apparatuses are water and ice baths, and coolers (chillers), for example, recirculating coolers which can be attached, for example, to the tanks used in the provided methods, for example, remotely or by a tank mounted in the cooler, to recirculate fluid from the tank, through the chiller and back to the tank, in order to rapidly cool and maintain the temperature of the mixture during mixing.

As used herein, acid addition salts of a compound are salts prepared by reacting the compound with an acid, such as an inorganic or organic acid. Exemplary acid addition salts of the aminoalkanes provided herein are acetic acid addition salts, hydrobromic acid addition salts, sulfuric acid addition salts, maleic acid addition salts, propionic acid addition salts and malonic acid addition salts and hydrochloric acid addition salts.

As used herein, a derivative of a compound refers to a substance having a chemical structure that is similar to the compound, yet containing a chemical group not present in the compound and/or deficient of a chemical group that is present in the compound. The compound to which the derivative is compared is known as the “parent” compound. The derivative can be physically derived from the parent compound or can be generated without beginning with the parent compound. A derivative shares at least one function with the parent compound from which it is derived, but differs from that compound structurally. Derivatives include, but are not limited to, salts, such as acid addition salts (e.g. HCl salts), aldehydes, amides, carbonate derivatives, esters, and acids of the parent compound. Typically, the derivatives of the aminoalkanes in the provided compositions are biocompatible derivatives, such as biocompatible salts, e.g. acid addition salts, carbonates, amides, aldehydes or other biocompatible derivatives.

As used herein, “biocompatible” refers to compounds that can safely be ingested by, or otherwise administered to, a subject, e.g. a human.

As used herein, a sugar substitute refers to a taste-modifying agent that is used in place of sugar to sweeten a composition; exemplary sugar substitutes are sucralose, dextrose, lactose, mannitol, sucrose, xylitol, malitol, acesulfame potassium, talin, glycyrrhizin, aspartame, saccharin, sodium saccharin, neotame, acesulfame potassium.

As used herein, an ampoule is a sealable vial that contains and/or preserves a fluid (e.g. liquid) composition. The ampoules are opened (and the liquids released) by breaking the seal on the ampoule, typically by applying pressure, e.g. by twisting or breaking the ampoule. Ampoules can be made of a variety of materials, such as glass or plastic or similar materials, such as soft plastics, such as low-density polymers such as thermoplastics, e.g. polyethylenes and polypropylenes, e.g. low-density polyethylene (LDPE). Ampoules include twist-top ampoules, which are opened by twisting the top of the ampoule to break the seal.

As used herein, “palatable” refers to compositions having pleasant (or not unpleasant), taste and/or smell and are desirable or at least tolerable for oral ingestion by a subject, e.g. a human. Palatability is used to express the degree of pleasant (or not unpleasant) taste and/or smell of a compound or composition compared to another compound or composition. For example, taste-modifying agents can improve the palatability of a composition, improving its taste and/or smell and its desirability for ingestion.

### B. COMPOSITIONS CONTAINING AMINOALKANES AND DERIVATIVES THEREOF COMPOSITIONS CONTAINING ACTIVE INGREDIENTS

Provided herein are compositions, e.g. palatable compositions, containing aminoalkanes and biocompatible derivatives thereof, and optionally other active ingredients. Provided are compositions containing high concentrations of the vasoconstrictor aminoalkane compounds and methods for making the compositions. The compositions are formulated for oral ingestion and include taste-modifying agents, e.g. flavoring agents, sweetening agents and other agents, that render them palatable. In particular, low volume compositions with high concentrations of the aminoalkanes are provided. Typically, the amounts of the active ingredients are effective to induce, promote or enhance one or more effects, such as upon delivery to a subject or upon administration to a sample, for example, syrnpathomimetic effects, stimulatory effects, vasoconstriction, decongestion (e.g. bronchial or nasal decongestion), increased energy, endurance, mood-enhancement, appetite suppression and/or weight loss. The amounts of the active ingredients typically are the highest possible amounts where the compositions are palatable, e.g. tolerated for oral administration and typically having a pleasant (or not unpleasant) taste and/or smell.

The active ingredients include vasoconstrictors, such as alpha-adrenergic agonists. For example, provided are compositions with active ingredients having equivalent vasoconstrictor activity compared to an amount of 2-amino-4-methylhexane or a salt thereof, such as 2-amino-4-methylhexane HCl, as described herein below. The equivalent activity can be equivalent activity per mass or per molecule of the compound, or it can be equivalent per volume of a composition containing the compound.

Vasoconstrictor activity can be determined by methods known to those skilled in the art, such as, but not limited to, methods described herein, below, e.g. section C(3) below (see, e.g. Swanson and Chen, Journal of Pharmacology and Experimental Therapeutics, 88(1), 10-13 (1946), citing Swanson et al., Journal of Pharmacology and Experimental Therapeutics, 85(1), 70-73 (1945), Swanson et al., Journal of Pharmacology and Experimental Therapeutics, 79(4), 329-333 (1943), and Elliott, J. Physiol. 44: 374-409 (1912)), where a test compound is injected intravenously into a pithed animal subject and blood pressure of the subject is measured; and Salem and Clemente, Arch Otolaryngology 96: 524-529 (1972) and McLeod et al., J Pharmacological and Toxicological Meth. 48: 153-159 (2002), where vasoconstrictor activity is measured with a rhinometric assay.

Typically, the provided compositions are low-volume compositions, which contain effective amounts of the active ingredients in relatively low volumes of liquid, such as, for example, less than at or about 5000, 4000, 3000, 2000, 1000, 900, 800, 700, 600 mL, and typically less than at or about 500, 450, 400, 350, 300, 250, 200, 150, 100, 75, 50, 40, 30, 20, 19, 18, 17, 16, 15, 14, 13, 12, 11, 10, 9, 8, 7, 6, 5, 4, 3, 2, 1, or fewer, mL, and typically are palatable compositions, which contain effective amounts of the active ingredients and are tolerable for oral administration, e.g. have pleasant, and/or not unpleasant, taste and/or smell. The palatable compositions are desirable or tolerable for consumption by a subject, e.g. by oral administration. For example, provided are low-volume palatable compositions containing effective amounts of active ingredients.

Also provided are combinations, articles of manufacture, and kits containing the compositions, methods for making the compositions, combinations, articles of manufacture and kits, and uses for the compositions, such as methods for providing active ingredients by delivery of the compositions, for example, by oral delivery of the compositions to a subject.

1. Active Ingredients

The active ingredients in the compositions include compounds having activities, such as promoting, causing and/or enhancing an effect in a sample and/or a subject, such as, but not limited to, sympathomimetic effects, stimulatory effects, vasoconstriction, decongestion (e.g. bronchial or nasal decongestion), increased energy, endurance, mood-enhancement, appetite suppression and/or weight loss. For example, provided are compounds with active ingredients (e.g. aminoalkanes and derivatives thereof) having vasoconstrictor activity that is equivalent to the vasoconstrictor activity (e.g. per weight or per molecule) of 2-amino-4-methylhexane and/or as compared to acid salts thereof, such as 2-amino-4-methylhexane HCl, and typically having equivalent vasoconstrictor activity to a particular amount (e.g. concentration, e.g. molarity) of 2-amino-4-methylhexane or salt thereof, such as 2-amino-4-methylhexane HCl. The active ingredients include, but are not limited to, sympathomimetics, vasoconstrictors and alpha-adrenergic agonists, such as sympathomimetic alpha-adrenergic agonists.

a. Aminoalkanes and Derivatives Thereof.

The active ingredients in the provided compositions include aminoalkanes and derivatives thereof, such as branched and substituted aminoalkanes and derivatives thereof, including compounds having a structure as set forth in Formula I:

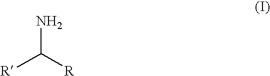

where R is selected from C₁-C₂₀ alkyl and R′ is hydrogen or C₁-C₂₀ alkyl, and analogs and derivatives thereof, including biocompatible salts, aldehydes and amides thereof, including carbonate derivatives, and esters and acid derivatives thereof, and particularly compounds of Formula I having vasoconstrictor activity that is equivalent or about equivalent, e.g. on a per molecule or per weight basis, to the vasoconstrictor activity of 2-amino-4-methylhexane and/or salts of 2-amino-4-methylhexane, such as 2-amino-4-methylhexane HCl, typically equivalent to the activity of a specified amount (e.g. concentration) of 2-amino-4-methylhexane HCl.

The aminoalkanes of Formula I include aminoalkanes with straight or branched R and/or R′ alkyl groups. For example, the alkyl groups of formula I can be straight chain alkyl groups, including but not limited to, methyl, ethyl, propyl, n-butyl and n-pentyl; and/or branched alkyl groups, including but not limited to, isopropyl, t-butyl and 2-methylbutyl.

In particular, the active ingredients include, but are not limited to, 2-aminoalkanes, such as branched and substituted 2-aminoalkanes, such as those having the structure set forth in formula II, below, and analogs and derivatives thereof, including biocompatible salts, aldehydes, amides thereof, carbonate derivatives, and ester and acid derivatives thereof, particularly those compounds and derivatives having vasoconstrictor activity that is equivalent or about equivalent to that of 2-amino-4-methylhexane and/or salts of 2-amino-4-methylhexane, such as 2-amino-4-methylhexane HCl or a specified amount of 2-amino-4-methylhexane HCl.

The 2-aminoalkane active ingredients include 2-aminoalkanes having between 3 and 22 carbon atoms, such as between 3 and 20, and typically between 3 and 15 or 3 and 10, such as 3, 4, 5, 6, 7, 8 or 9 carbon atoms, typically between 4 and 9, or 6 and 9 carbon atoms, particularly those compounds having vasoconstrictor activity that is equivalent or about equivalent to that of 2-amino-4-methylhexane and/or salts of 2-amino-4-methylhexane, such as 2-amino-4-methylhexane HCl or a specified amount of 2-amino-4-methylhexane HCl. For example, the 2-aminoalkanes include aminoalkanes having structures set forth in Formula II:

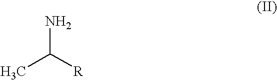

where R is selected from C₁-C₂₀ alkyl, and typically is selected from C₂-C₂₀ alkyl,

including 2-aminoalkanes having between 4 and 9 carbon atoms, such as any of the aminoalkanes having structures as set forth in Formula III:

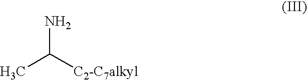

typically 2-aminoalkanes having between 6 and 9, carbon atoms, such as any of the aminoalkanes having the structures set forth in Formula IV:

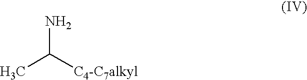

The 2-aminoalkane active ingredients include 2-aminoalkanes having between 3 and 22 carbon atoms, such as between 3 and 20, or between 3 and 15 or between 3 and 10 carbon atoms, and typically between 4 and 9 carbon atoms or between 6 and 9 carbon atoms, such as 2-aminoalkanes containing one or more methyl groups, such as, but not limited to, 2-amino-4-methylhexane (also known as 1,3-dimethylamylamine, 1,3-dimethylpentylamine, 2-amino-4-methylhexane, 4-methyl-2-hexylamine, methylhexaneamine, AMH, Forthane and 4-methyl-2-hexanamine), 2-aminoheptane, 2-amino-4-methylheptane, 3-methylhexan-2-amine, 5-methylhexan-2-amine, 3-ethylhexan-2-amine, 5 and 4-methylhexane-2,5-diamine, and analogs and derivatives thereof, including biocompatible salts, aldehydes and amides thereof, including carbonate derivatives and ester and acid derivatives thereof, particularly those compounds having vasoconstrictor activity that is equivalent or about equivalent to that of 2-amino-4-methylhexane and/or salts of 2-amino-4-methylhexane, such as 2-amino-4-methylhexane HCl or a specified amount of 2-amino-4-methylhexane HCl.

In particular, the active ingredients include 2-amino-4-methylhexane and acid salts and derivatives thereof, such as 2-amino-4-methylhexane HCl and other acid salts having equivalent or about equivalent vasoconstrictor activity, such as 2-amino-4-methylhexane HCl (1,3-dimethyl-pentylamine HCl) distributed by Dishman Pharmaceuticals and Chemicals, Limited, Jiangsu, China, Best Line Botanicals Co., Ltd. High-Tech Industries Development Zone, Xi'an, P. R. China; or Allway, International, China, particularly Kosher certified 2-amino-4-methylhexane HCl.

Exemplary of the aminoalkane derivatives are aldehyde derivatives, such as, but not limited to, aldehydes of 2-aminoalkanes, having the structure set forth in Formula V:

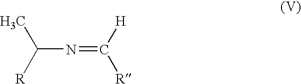

where R is C₁-C₂₀ alkyl, and R″ is selected from C₁-C₂₀ alkyl or a monocyclic aromatic group,

including aldehyde derivatives of 2-amino-4-methylhexane and aldehyde derivatives having equivalent or about equivalent vasoconstrictor activity.

The aldehyde derivatives of aminoalkanes, e.g. of 2-aminoalkanes, can be prepared by methods known in the art. For example, aldehyde derivatives can be made by reacting aminoalkanes with aldehydes, such as, but not limited to, benzaldehyde, propionaldehyde, n-butyraldehyde, acetaldehyde, isobutyraldehyde, propionaldehyde (see, for example, U.S. Pat. No. 2,455,193). The aldehyde derivatives include, but are not limited to, 2-benzalaminohexane, 2-propionalaminoheptane, 2-ethanalaminoheptane, 2-propionalamino-4-methylhexane, 2-benzalamino-5-methylhexane, 2-isobutanol-amino-4-methylheptane, 2-propionalamino-6-methylheptane, 2-propionalamino-4,6,-dimethylheptane and 2-propionalamino-4-methylhexane.

Aldehyde derivatives of Formula V (particularly aldehyde derivatives having vasoconstrictor activity that is equivalent or about equivalent to that of 2-amino-4-methylhexane and/or salts of 2-amino-4-methylhexane), can be prepared by methods known in the art (see, for example, U.S. Pat. No. 2,455,193). For example, reaction of an aminoalkane, such as set forth in Formula I, II, III or IV, with an aldehyde, followed by removal of the water formed during the reaction by distillation, provides the corresponding aldehyde derivative. Using this method, 2-aminohexane is cooled to 0° C. and mixed with benzaldehyde that also is cooled to 0° C. Heat evolves from the reaction. The water that is formed separates from the mixture and is removed, e.g. with a separatory funnel. The resulting mixture is dried over anhydrous magnesium sulfate and distilled in vacuo to produce 2-benzalaminohexane.

The aminoalkane derivatives also include biocompatible salts of aminoalkanes (e.g. 2-aminoalkanes), such as a salt of a compound of formula I, II, III IV or V, particularly 2-amino-4-methylhexane HCl and salts having a comparable vasoconstrictor activity to 2-amino-4-methylhexane HCl. Salts of the aminoalkanes can be obtained using standard procedures well known in the art. For example, an acceptable salt can be prepared by treatment of the aminoalkane with an inorganic or organic acid. For example, suitable salts can be formed by treatment of the aminoalkane with an organic acid, such as acetic acid, adamantanecarboxylic acid, adipic acid, ascorbic acid, aspartic acid, azelaic acid, benzoic acid, 2-(4-hydroxybenzoyl)benzoic acid, carboxylic acid, cinnamic acid, citric acid, cyclohexanecarboxylic acid, decanoic acid, dodecanoic acid, 1,2-ethanedisulphonic acid, ethanesulphonic acid, ethylenediaminetetraacetic acid (EDTA), fumaric acid, glucoheptonic acid, gluconic acid, glutamic acid, glycolic acid, hippuric acid, lactic acid, lactobionic acid, maleic acid, hydroxymaleic acid, methylmaleic acid, malic acid, malonic acid, mandelic acid, 1-hydroxy-2-naphthoic acid, 3-hydroxy-2-naphthoic acid, octanoic acid, pamoic acid, pantothenic acid, phthalic acid, phenylacetic acid, pimelic acid, phosphonic acid, propionic acid, 2-(4-chlorophenoxy)-2-methylpropionic acid, salicylic acid, 4-aminosalicylic acid, stearic acid, suberic acid, succinic acid, sulfamic acid, N-cyclohexylsulfamic acid, N-methyl-, N-ethyl-, or N-propyl-sulfamic acid, sulfonic acid, benzenesulfonic acid, disulfonic acid, ethane-1,2-disulfonic acid, 2-hydroxyethanesulfonic acid, methane- or ethane-sulfonic acid, 2-, 3- or 4-methylbenzenesulfonic acid, 2-naphthalenesulfonic acid, 1,5-naphthalene-disulfonic acid, p-toluenesulphonic acid, methylsulfuric acid, ethylsulfuric acid, dodecylsulfuric acid, tannic acid, tartaric acid, terephthalic acid, and 10-undecenoic acid and other organic protonic acids.

Alternatively, the salts can be inorganic acid addition salts of the aminoalkanes, such as, but not limited to, bicarbonates, carbonates, chlorides, bromides, iodides, nitrates, perchlorates, phosphates, monohydrogenphosphates, dihydrogenphosphates, metaphosphates, pyrophosphates, sulfates, pyrosulfates, bisulfates, sulfites, and bisulfites, hydrochloric acid addition salts, sulfuric acid addition salts, and hydrobromic acid addition salts. Thus, exemplary aminoalkane acid addition salts include, but not limited to, acetic acid addition salts, hydrobromic acid addition salts, sulfuric acid addition salts, maleic acid addition salts, propionic acid addition salts and malonic acid addition salts and hydrochloric acid addition salts, including, but not limited to, 2-amino-4-methylhexane HCl (also called 1,3-dimethylamylamine HCl, 1,3-dimethylpentylamine HCl, 2-amino-4-methylhexane HCl, 4-methyl-2-hexylamine HCl, methylhexaneamine HCl, AMH HCl, Forthane HCl and 4-methyl-2-hexanamine HCl).

The aminoalkane salts also include carbonates derivatives of 2-aminoalkanes, such as carbonates of Formula VI:

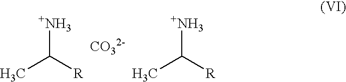

where R is a C₁-C₂₀ alkyl group (see, for example, U.S. Pat. No. 2,386,273), particularly carbonate derivatives of 2-amino-4-methylhexane and carbonate derivatives having equivalent or about equivalent vasoconstrictor activity.

The carbonate derivatives include, but are not limited to, 2-aminoheptane carbonate, 2-amino-4-methylhexane carbonate and 2-amino-4-methylheptane carbonate.

Aminoalkane salts of Formula VI can be prepared by methods known in the art (see, for example, U.S. Pat. No. 2,386,273). For example, an aminoalkane, such as one of Formula I, II, III or IV, particularly one having at or about an equivalent vasoconstrictor activity to 2-amino-4-methylhexane or 2-amino-4-methylhexane HCl, is dissolved in dry diethyl ether and cooled with an ice bath. A fairly rapid stream of moist carbon dioxide is passed into the solution. After the reaction has been completed, the ether is removed by evaporation in vacuo at room temperature to produce the corresponding aminoalkane carbonate.

In particular, the active ingredients in the provided compositions include 2-amino-4-methylhexane, which is a compound of Formula I (where R is 2-methylbutane and R′ is methyl) or of Formula II, III or IV (where R is 2-methylbutane), having the structure:

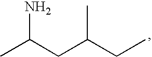

and biocompatible analogs and derivatives thereof, including biocompatible salts, aldehydes and amides thereof, carbonate derivatives and acid and ester derivatives thereof, particularly derivatives having at or about an equivalent vasoconstrictor activity to 2-amino-4-methylhexane or 2-amino-4-methylhexane HCl.

In particular, the 2-amino-4-methylhexane derivatives include 2-amino-4-methylhexane HCl (the hydrochloric acid addition salt of 2-amino-4-methylhexane; see, for example, U.S. Pat. No. 2,350,318), and can include other acid addition salts and derivatives of 2-amino-4-methylhexane, particularly with equivalent or about equivalent vasoconstrictor activity.

### (i) Vasoconstrictor Activity

The aminoalkane and aminoalkane derivatives in the compositions active include such compounds having vasoconstrictor (pressor) activity, such as alpha-adrenergic agonists, including 2-aminoalkanes. The vasoconstrictor activity typically is compared to 2-amino-4-methylhexane or 2-amino-4-methylhexane HCl, for example, a particular amount (e.g. concentration) of 2-amino-4-methylhexane or 2-amino-4-methylhexane HCl, and its concentration adjusted to be equivalent to the specified amount of 2-amino-4-methylhexane HCl.

For example, the compositions contain aminoalkanes and/or aminoalkane derivatives at an amount having a vasoconstrictor activity equivalent to the vasoconstrictor activity of 2-amino-4-methylhexane HCl at a concentration of between at or about 3 millimoles per liter (mM) and at or about 1000 mM, between at or about 3 mM and at or about 500 mM, or between at or about 3 mM and at or about 250 mM, or between at or about 3 mM and 200 mM, such as 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 155, 160, 165, 170, 175, 180, 185, 190, 195 or 200, such as between at or about 5 mM and at or about 150 mM, e.g. between at or about 5 mM and at or about 110 mM, such as between at or about 5 mM and at or about 110 mM, e.g. at or about 107 mM; or at an amount that has a vasoconstrictor activity equivalent to the vasoconstrictor activity of 2-amino-4-methylhexane HCl at a concentration of between at or about 1 mg/mL and at or about 100 mg/mL, typically between at or about 1 mg/mL and at or about 50 mg/mL, typically between at or about 1 mg/mL and 30 mg/mL, e.g. at or about 1, 2, 3, 4, 5, 6, 7, 8, 9, 10, 11, 12, 13, 14, 15, 16, 16.25, 16.5, 17, 18, 19, 20, 21, 22, 23, 24, 25, 26, 27, 28, 29, 30, 31, 32, 33, 34 or 35 mg/mL, such as between at or about 15 mg/mL and 30 mg/mL; or at an amount that has a vasoconstrictor activity equivalent to the vasoconstrictor activity of between at or about 100 and 1000 micromoles (μmoles) of 2-amino-4-methylhexane HCl, e.g. 100, 110, 120, 130, 140, 150, 160, 170, 180, 190, 200, 225, 250, 275, 280, 290, 300, 310, 320, 330, 340, 350, 360, 370, 380, 390, 400, 425, 426, 427, 428, 429, 430, 450, 500, 600, 700, 800, 900 or more micromoles, or between at or about 5 g and 200 g, e.g. at or about 5, 6, 7, 8, 9, 10, 15, 16, 17, 18, 19, 20, 21, 22, 23, 24, 25, 26, 27, 28, 29, 30, 40, 50, 55, 60, 65, 70, 75, 80, 85, 90, 95, 100, 125, 150 or 200 g, or at least at or about 15, 16, 17, 18, 19, 20, 21, 22, 23, 24, 25, 26, 27, 28, 29, 30, 40, 50, 55, 60, 65, 70, 75, 80, 85, 90, 95, 100, 125, 150 or 200 g, such as at or about 65 g 2-amino-4-methylhexane HCl, for example, in a volume of between at or about 1 and 10 mL, typically between 2 and 5 mL, such as 4 mL.

In other examples, the compositions contain aminoalkanes or derivatives thereof at a concentration with the highest possible vasoconstrictor activity where the composition is a palatable composition.

Vasoconstrictor activity, including whether two compounds have equivalent vasoconstrictor activity and/or the amount of a test compound having equivalent vasoconstrictor activity to another compound, can be determined by assays known in the art, including assays that measure vasoconstrictor activity of vasoconstrictors such as epinephrine or adrenaline, such as those assays that have been used to measure the vasoconstrictor activity of aminoalkanes, e.g. 2-aminoalkanes (see Swanson and Chen, Journal of Pharmacology and Experimental Therapeutics, 88(1), 10-13 (1946), citing Swanson et al., Journal of Pharmacology and Experimental Therapeutics, 85(1), 70-73 (1945) and Swanson et al., Journal of Pharmacology and Experimental Therapeutics, 79(4), 329-333 (1943)). Such assays include, for example, evaluation of vasoconstriction in animal subjects as described in Elliott, J. Physiol. 44: 374-409 (1912), where a test compound is injected intravenously into a pithed animal subject and blood pressure of the subject is measured. Animal subjects can include, but are not limited to, cats, dogs, guinea pigs, rabbits, primates, frogs, rats and mice. Blood pressure is measured before and after administration of the compound and vasoconstrictor activity of the test compound is determined as a function of the change in blood pressure.

In one variation, an animal (e.g. cat or dog) is pithed according to methods known in the art (e.g. anesthetized and its brain destroyed by insertion of a probe upward through the foramen magnum at the base of the skull). Artificial respiration is then induced following insertion of a tracheal cannula. After a short delay, a probe is inserted into the orbit and routed through the cranial cavity and down the spinal canal to about the fourth thoracic segment. After the blood pressure stabilizes, the circulatory system is ready for the assay. The test compound is injected into the external jugular vein at various doses and at various intervals. Blood pressure measurements are taken and plotted on a graph to determine the activity of the compound.

Generally, a rise in blood pressure is indicative of an active vasoconstriction compound. The vasoconstrictor activity typically is assessed by comparing change in blood pressure following administration of the test compound to change in blood pressure following administration (e.g. in the same animal), of a dose, and typically a range of doses, of a standard compound, such as epinephrine or adrenaline, or an aminoalkane, e.g. 2-amino-4-methylhexane or 2-amino-4-methylhexane HCl.

Alternatively, vasoconstriction activity can be measured using a rhinometric assay. In this type of assay, changes in the nasal cavity patency (i.e. degree of nasal cavity clearance or blockage) are evaluated in the presence of a test compound. Animal subjects for use in the rhinometric assay include, but are not limited to, rats, guinea pigs, rabbits, cats, dogs and pigs. Rhinometric assays include assays where nasal cavity geometry is measured, such as in acoustic rhinometry (see e.g., Rinder and Lundberg, Acta Physiol Scand 157: 233-244 (1996)). The assays also include those where nasal resistance values are derived from measurements of airflow and differential pressures between proximal and distal points within the nasal cavity (see e.g., Salem and Clemente, Arch Otolaryngology 96: 524-529 (1972) and McLeod et al., J Pharmacological and Toxicological Meth. 48: 153-159 (2002)). In one variation of the rhinometric assay, an allergen, such as ovalbumin, is administered to an animal subject, such as a guinea pig, in the presence or absence of a test compound, and changes in nasal patency are assessed based on measurements of forced airflow across the nasal cavity. Typically, the esophagus, mouth and one nostril are sealed in order to precisely direct the airflow generated from a tracheal cannula through the single open nasal passage. Pressure changes across the cavity due to the forced airflow can be measured using a physiological pressure transducer. Nasal pressure values can be converted to nasal resistance using the following formula: resistance=Δ pressure/flow. Activity of the test compound is determined as a function of the change in nasal resistance when the subject is challenged with an allergen. Generally, an active vasoconstrictor compound will decrease the degree of nasal resistance during an allergen challenge.

With the vasoconstrictor assays, the vasoconstrictor activity can be expressed by comparison to a standard compound (e.g. an epinephrine equivalent, see Swanson and Chen, Journal of Pharmacology and Experimental Therapeutics, 88(1), 10-13 (1946)). The values (e.g. change in blood pressure or nasal resistance) obtained in the assays with the test compounds are compared to values obtained, e.g. in the same animal subjects, with an amount, and typically a range of amounts, of a standard compound, such as one with a known vasoconstrictor activity, e.g. epinephrine, or a 2-aminoalkane, such as 2-amino-4-methylhexane or 2-amino-4-methylhexane HCl. In this example, the vasoconstrictor activity can be expressed as an equivalent of a known amount of the standard compound, such as an epinephrine equivalent, which is the amount of the standard compound that has equivalent activity to a given amount (e.g. 1 mg or 1 mole or 100 mM) of the test compound. For example, the average epinephrine equivalent can be listed for 1 mg of a plurality of different aminoalkanes, where the epinephrine equivalent is the amount of epinephrine that contains the equivalent vasoconstrictor activity to 1 mg of the aminoalkane (see, for example, Swanson and Chen, Journal of Pharmacology and Experimental Therapeutics, 88(1), 10-13 (1946)).

For example, one assay for vasoconstrictor activity revealed the following average epinephrine equivalents (per 1 mg of sulfate derivatives of the following aminoalkanes):

| | Epinephrine equivalent (micrograms |
|---|---|
| | (μg)) of 1 mg of the sulfate salt of the |
| Aminoalkane | aminoalkane |
| 1-amino-3-methyl butane | 0.238 |
| 1-amino-pentane | 0.210 |
| 1-amino-3-methyl pentane | 0.193 |
| 1-amino-4-methyl pentane | 0.530 |
| 1-amino-3,4-dimethylpentane | 1.720 |
| 2-amino-pentane | 0.395 |
| 2-amino-4-methylpentane | 0.840 |
| 2-amino-4,4-dimethylpentane | 0.083 |
| 1-amino-hexane | 0.766 |
| 1-amino-3-methylhexane | 0.108 |
| 1-amino-3,5-dimethylhexane | 0.660 |
| 2-aminohexane | 0.901 |
| 2-amino-3-methylhexane | 0.858 |
| 2-amino-4-methylhexane | 3.520 |
| 2-amino-5-methylhexane | 1.560 |
| 2-amino-hexene-5 | 0.193 |
| 2-amino-5-methylhexene-5 | 0.504 |
| 1-amino-heptane | 0.276 |
| 1-amino-3-methylheptane | 1.230 |
| 2-aminoheptane | 3.100 |
| 2-amino-3-methylheptane | 0.530 |
| 2-amino-4-methylheptane | 0.786 |
| 2-amino-5-methylheptane | 0.980 |
| 2-amino-6-methylheptane | 2.620 |
| 2-amino-3,6-dimethylheptane | 0.910 |
| 2-amino-4,6-dimethylheptane | 0.193 |
| 2-amino-5-ethylheptane | 0.358 |
| 2-amino-4,6,6-trimethylheptane | nil |
| 3-aminoheptane | 0.149 |
| 3-amino-6-methylheptane | nil |
| 4-aminoheptane | 0.048 |
| 4-amino-2,6-dimethylheptane | nil |
| 2-amino-6-methylheptene-5 | 0.391 |
| 1-amino-octane | nil |
| 2-amino-octane | 0.510 |
| 2-amino-4-methyloctane | 0.475 |
| 2-amino-3-hydroxy-octane | 0.122 |
| 3-amino-octane | 0.142 |
| 2-amino-4-methyldecane | 0.242 |

(Swanson and Chen, Journal of Pharmacology and Experimental Therapeutics, 88(1), 10-13 (1946)).

Studies further have shown that 2-aminoalkanes having from 6 to 9 carbon atoms have vasoconstrictor activity and that aldehyde derivatives of these aminoalkanes have equivalent or greater vasoconstrictor activity on a per weight basis (see U.S. Pat. No. 2,455,193), that 2-amino-4-methylhexane and its acid addition salts (e.g. acetic, hydrobromic, hydrochloric, sulfuric, maleic, propionic and maleic acid addition salts) have greater vasoconstrictor activity compared to ephedrine or amphetamine (see U.S. Pat. No. 2,350,318) and that carbonate derivatives of 2-aminoalkanes have comparable vasoconstrictor activity (see, for example, U.S. Pat. No. 2,386,273).

Thus, determining an amount of a first compound, such as an active ingredient (e.g. aminoalkane or biocompatible derivative), that has equivalent vasoconstrictor activity to a specified amount of a second compound (e.g. 2-aminoalkane-4-methylhexane HCl) can be done by comparing one or more amounts (e.g. mass, quantity or concentration) of the first compound to the specified amount (e.g. mass, quantity or concentration) of the second compound in a vasoconstrictor assay; or can be determined by first determining the standard compound equivalent (e.g. the epinephrine equivalent) of an amount of each compound in a vasoconstrictor assay, and then determining the amount of the first compound that is needed to equal the same standard compound (e.g. epinephrine) equivalent to the specific amount of the second compound (e.g. 2-amino-4-methylhexane HCl).

(ii) Amounts and Activity of the Aminoalkane and Aminoalkane Derivative Active Ingredients

Provided are compositions containing effective amounts of aminoalkanes and aminoalkane derivatives, the effective amounts being sufficient to produce, promote or enhance one or more effects upon delivery to a subject, such as stimulant and/or decongestant effect, vasoconstriction, appetite suppression, mood-enhancement, increased energy and/or weight loss, or an amount necessary for preventing, curing, ameliorating, arresting or partially arresting a symptom of a disease or disorder, or the quantity and/or concentration desired by an individual for intake, such as daily intake, and/or nutritional supplementation, for example, an amount sufficient to enhance the nutritional, pharmaceutical, nutraceutical, health or energy property of a food, beverage, or other consumable.

The amount of the aminoalkane or derivative thereof is typically the highest possible amount (e.g. concentration) of the aminoalkane (or the amount providing the highest amount of vasoconstrictor activity) that yields a palatable composition, and is typically between at or about 3 mM and at or about 1000 mM, for example, at or about 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 155, 160, 165, 170, 175, 180, 185, 190, 195, 196, 197, 198, 199, 200, 210, 220, 230, 240, 250, 260, 270, 280, 290, 300, 350, 400, 500 or 1000 mM, typically between at or about 3 mM and at or about 500 mM, such as between at or about 3 mM and at or about 250 mM, or between at or about 3 mM and at or about 200 mM, and typically between at or about 3 mM and at or about 150 mM, typically between at or about 3 mM and at or about 120 mM, such as between at or about 3 mM and at or about 110 mM, e.g. between at or about 100 mM and at or about 110 mM, e.g. at or about 107 mM, or between at or about 1 mg/mL and at or about 100 mg/mL, typically between at or about 1 mg/mL and 50 mg/mL or between at or about 1 mg/mL and 35 mg/mL, e.g. at or about 1, 2, 3, 4, 5, 6, 7, 8, 9, 10, 11, 12, 13, 14, 15, 16, 16.25, 16.5, 17, 18, 19, 20, 21, 22, 23, 24, 25, 26, 27, 28, 29, 30, 31, 32, 33, 34 or 35 mg/mL.

The appropriate amount of the aminoalkane active ingredient can be determined empirically, e.g. by generating a plurality of compositions containing a range of amounts and determining the highest possible amount that yields a palatable composition, e.g. by tasting/smelling or otherwise empirically analyzing the compositions.

The amount (e.g. mass, quantity or concentration) of the aminoalkane (or aminoalkane derivative) active ingredient also can be determined based on the activity, typically vasoconstrictor (pressor) activity, for example, to provide an amount of the active ingredient having a vasoconstrictor activity that is equivalent to the vasoconstrictor activity of a specified concentration of 2-amino-4-methylhexane HCl.

For example, provided are palatable compositions containing an aminoalkane or derivative thereof at a concentration having a vasoconstrictor activity equivalent to the vasoconstrictor activity of 2-amino-4-methylhexane HCl at a concentration of between at or about 3 mM and at or about 1000 mM, such as at or about 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 155, 160, 165, 170, 175, 180, 185, 190, 195, 196, 197, 198, 199, 200, 210, 220, 230, 240, 250, 260, 270, 280, 290, 300, 350, 400, 500 or 1000 mM, and typically between at or about 3 mM and at or about 200 mM, for example, at or about such as between at or about 5 mM and 150 mM, e.g. between 25 mM and 110 mM, e.g. at or about 107 mM.

In another example, the amount of the aminoalkane or aminoalkane derivative in the composition is between at or about 100 and 1000 micromoles, e.g. at or about 100, 110, 120, 130, 140, 150, 160, 170, 180, 190, 200, 225, 250, 275, 280, 290, 300, 310, 320, 330, 340, 350, 360, 370, 380, 390, 400, 425, 426, 427, 428, 429, 430, 450, 500, 600, 700, 800, 900 or more micromoles; or between at or about 15 g and 200 g, e.g. 15, 16, 17, 18, 19, 20, 21, 22, 23, 24, 25, 26, 27, 28, 29, 30, 40, 50, 55, 60, 65, 70, 75, 80, 85, 90, 95, 100, 110, 120, 130, 140, 150 or 200 g, for example, at or about 65 g, for example, in a volume of between at or about 1 and 10 mL, typically between 2 and 5 mL, such as 4 mL.

In another example, the amount of the aminoalkane or aminoalkane derivative is an amount having a vasoconstrictor activity equivalent to the vasoconstrictor activity of between at or about 100 and 1000 micromoles of 2-amino-4-methylhexane HCl; e.g. at or about 100, 110, 120, 130, 140, 150, 160, 170, 180, 190, 200, 225, 250, 275, 280, 290, 300, 310, 320, 330, 340, 350, 360, 370, 380, 390, 400, 425, 426, 427, 428, 429, 430, 450, 500, 600, 700, 800, 900 or more, or between at or about 15 g and 200 g, e.g. at or about 15, 16, 17, 18, 19, 20, 21, 22, 23, 24, 25, 26, 27, 28, 29, 30, 40, 50, 55, 60, 65, 70, 75, 80, 85, 90, 95, 100, 110, 120, 125, 130, 140, 150 or 200 g, for example, at or about 65 g 2-amino-4-methylhexane HCl, for example, in a volume of between at or about 1 and 10 mL, typically between 2 and 5 mL, such as 4 mL.

In another example, the amount of the aminoalkane or aminoalkane derivative can be in the composition at an amount of between at or about 0.01% and at or about 50% and typically is in the composition at between 0.01% and 25% or between at or about 0.1% and at or about 25%, by weight, such as, for example, 0.1%, 0.2%, 0.3%, 0.4%, 0.5%, 0.6%, 0.7%, 0.8%, 0.9%, 1%, 1.1%, 1.2%, 1.3%, 1.4%, 1.5%, 1.6%, 1.625%, 1.65%, 1.7%, 1.8%, 1.9%, 2.0%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, or more, by weight, of the composition.

b. Other Active Ingredients

The compositions further can include one or more additional active ingredients, such as other active ingredients to promote a biologic effect, such as, for example, stimulant and/or decongestant effect, vasoconstriction, appetite suppression, mood-enhancement, increased energy and/or weight loss, improved health or other biologic effect. In one example, the other active ingredients are selected from among stimulants, nutraceuticals, minerals, vitamins, amino acids, fatty acids, electrolytes, sports nutritional products, probiotics, metabolites, antioxidants, hormones, enzymes, cartilage products, botanical extracts, homeopathic products, dietary supplements such as weight-loss promoting compounds, such as ingredients selected from among any one or more of Vitamin B12, chromium picolinate, caffeine (such as caffeine added to the composition in the form of caffeine anhydrous), conjugated linoleic acid (CLA), L-taurine and alpha lipoic acid. Any combination of such agents can be selected for use in the compositions provided.

The amount of the other active ingredients in the composition can be between at or about 0% and at or about 50%, by weight, and typically is between at or about 0% and at or about 25%, by weight, such as between at or about 0.015% and at or about 25%, by weight, e.g. at or about 0.015%, 0.025%, 0.03%, 0.04%, 0.05%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24% or 25%, by weight, and typically between at or about 0.015% and at or about 10%, or between at or about 0.015% and at or about 4%, by weight of the composition, or between at or about 1.625% and at or about 2.3%, by weight, of the composition, e.g. at or about, or less than at or about 5, 4, 3, 2, 1.9, 1.8, 1.7, 1.6, 1.5, 1.4, 1.3, 1.2, 1.1, 1, 0.9, 0.8, 0.7, 0.6, 0.5, 0.4, 0.3, 0.2, 0.1%, or less, by weight, of the composition.

The CLA active ingredients include a CLA compound (80% CLA) sold commercially by Sanmark, LTD (Dalian, Liaoning Province, China; product code 01057-A80), which is clear white to pale yellow oil and has the following fatty acid composition: NMT (not more than) 9.0% C16:0 Palmitic acid, NMT 4.0% Stearic acid, NMT 15.0% C18:1 Oleic acid, NMT 3.0% C18:2 Linoleic acid, NLT (not less than) 80% C18:2 CLA (including the following isomers: NLT 37.5% C18:2 CLA c9,t11, 37.5% C18:2 CLA t10, c12, and NMT 5.0% other CLA isomers); and NMT 5.0% other fatty acids; and the CLA compound sold under the trade name Tonalin®, by Cognis Corporation, Cincinnati, Ohio, which contains 1.7%, by weight, C16:0 Palmitic acid, 2.6%, by weight, C: 18 Stearic acid, 13.00% C18:1 C9 Oleic acid, 0.20%, by weight, C18:2 C9 C12 Linoleic acid and 81.00%, by weight, conjugated linoleic acid (CLA), which includes 39.70% Conjugated C9, T11 isomer and 39.50% Conjugated T10, C12 isomer). Other CLA containing compounds can be used in the compositions.

Typically, the amount of the CLA active ingredient is less than at or about 1%, by weight, of the composition, e.g. between at or about 0% and 1%, typically between at or about 0% and 0.5%, typically between at or about 0% and 0.3%, e.g. between at or about 0% and 0.25%, such as at or about 0.248%, by weight of the composition.

The Vitamin B12 active ingredients include any Vitamin B12, such as Vitamin B12 powders, including the pseudo Cyanocobalamin Vitamin B12 powder sold by Hebei Yuxing Bio-engineering Co., LTD, China, which is a dark red crystalline powder containing food-grade Vitamin B12, or other Vitamin B12 powders sold by this or other companies, particularly food-grade Vitamin B12. The amount of the Vitamin B12 active ingredient can be between at or about 0% and at or about 50%, by weight, of the composition, and typically is between at or about 0% and at or about 25%, and typically between at or about 0% and at or about 10% or between at or about 0% and at or about 5%, e.g. at or about 0%, 0.01%, 0.015%, 0.02%, 0.025%, 0.03%, 0.035%, 0.04%, 0.05%, 0.06%, 0.07%, 0.08%, 0.09%, 0.1%, 0.15%, 0.2%, 0.3%, 0.4%, 0.5%, 0.6%, 0.7%, 0.8%, 0.9%, 1% 1.5%, 2%, 2.5%, 3%, 3.5%, 4%, 4.5%, 5%, 6%, 7%, 8%, 8%, 9%, 10%, such as between at or about 0% and at or about 1%, by weight, of the composition, e.g. between at or about 0% and 1%, e.g. between at or about 0% and 0.03%, e.g. between at or about 0% and 0.025%, such as at or about 0.025%, by weight of the composition.

The caffeine active ingredients include caffeine that is added in the form of caffeine anhydrous, such as the Caffeine Anhydrous powder (white, crystalline powder), sold by Pacific Rainbow International, Inc., City of Industry, Calif., which is a white crystalline powder containing caffeine anhydrous. The amount of caffeine in the composition can be between at or about 0% and at or about 50%, by weight, of the composition, and typically is between at or about 0% and at or about 25%, and typically between at or about 0% and at or about 10%, or between at or about 0% and at or about 5%, e.g. at or about 0%, 0.01%, 0.015%, 0.02%, 0.025%, 0.03%, 0.035%, 0.04%, 0.05%, 0.06%, 0.07%, 0.08%, 0.09%, 0.1%, 0.15%, 0.2%, 0.3%, 0.4%, 0.5%, 0.6%, 0.7%, 0.8%, 0.9%, 1% 1.5%, 2%, 2.5%, 3%, 3.5%, 4%, 4.5%, 5%, 6%, 7%, 8%, 9%, 10%, such as between at or about 0% and at or about 3%, by weight, of the composition, e.g. between at or about 0% and 2%, e.g. at or about 2%, by weight, of the composition, or is less than 5%, by weight, of the composition, e.g. at or about 5, 4, 3, 2 or 1%, by weight, of the composition, or less. In one example, the composition contains at or about 2%, by weight, caffeine. In another example, the composition contains between at or about 1 and at or about 500 mg caffeine per mL or per serving, such as a 4 mL serving of the composition, e.g. at or about 200, 150, 125, 100, 80, 75, 50 or 25 milligrams (mg) caffeine per serving of the composition, e.g. per 4 mL of the composition.

The chromium picolinate active ingredients include Bio Chromium Picolinate, distributed by Stauber Performance Ingredients, Inc., Fullerton, Calif. (item # 21243) and manufactured by Kelatron Corporation, Ogden, Utah and other chromium picolinates. The amount of the chromium picolinate active ingredient can be between at or about 0% and at or about 50%, by weight, of the composition, and typically is between at or about 0% and at or about 25%, and typically between at or about 0% and at or about 10% or between at or about 0% and at or about 5%, e.g. at or about 0%, 0.01%, 0.015%, 0.02%, 0.025%, 0.03%, 0.035%, 0.04%, 0.05%, 0.06%, 0.07%, 0.08%, 0.09%, 0.1%, 0.15%, 0.2%, 0.3%, 0.4%, 0.5%, 0.6%, 0.7%, 0.8%, 0.9%, 1%1.5%, 2%, 2.5%, 3%, 3.5%, 4%, 4.5%, 5%, 6%, 7%, 8%, 9%, 10%, by weight, such as between at or about 0% and at or about 1%, by weight, of the composition, e.g. between at or about 0% and 1%, e.g. between at or about 0% and 0.02%, e.g. between at or about 0% and 0.015%, such as at or about 0.015%, by weight of the composition.

The L-taurine active ingredients include Taurine, sold by DNP International Co., Inc., Santa Fe Springs, Calif., and Taurine, manufactured by Qianjiang Yongan Pharmaceutical Co., Ltd., distributed by Stauber Performance Ingredients, Inc., Fullerton, Calif., catalog number 21195 and other L-taurines. The amount of L-taurine active ingredients can be between at or about 0% and at or about 50%, by weight, of the composition, and typically is between at or about 0% and at or about 25%, and typically between at or about 0% and at or about 10% or between at or about 0% and at or about 5%, e.g. at or about 0%, 0.01%, 0.015%, 0.02%, 0.025%, 0.03%, 0.035%, 0.04%, 0.05%, 0.06%, 0.07%, 0.08%, 0.09%, 0.1%, 0.15%, 0.2%, 0.25%, 0.3%, 0.4%, 0.5%, 0.6%, 0.7%, 0.8%, 0.9%, 1% 1.5%, 2%, 2.5%, 3%, 3.5%, 4%, 4.5%, 5%, 6%, 7%, 8%, 9%, 10%, by weight, such as between at or about 0% and at or about 1%, by weight, of the composition, e.g. between at or about 0% and 1%, e.g. between at or about 0% and 0.5%, e.g. between at or about 0% and 0.3%, e.g. between at or about 0% and 0.250%, such as at or about 0.250%, by weight of the composition.

The alpha lipoic acid active ingredients include Alpha Lipoic Acid, sold by NutriChem Resources Company, Walnut, Calif., and Alpha Lipoic Acid, sold by Zhejiang Medicines & Health Products Import & Export Co., Ltd, Hangzhou, China and other alpha lipoic acids. The amount of alpha lipoic acid active ingredients can be between at or about 0% and at or about 50%, by weight, of the composition, and typically is between at or about 0% and at or about 25%, and typically between at or about 0% and at or about 10% or between at or about 0% and at or about 5%, e.g. at or about 0%, 0.01%, 0.015%, 0.02%, 0.025%, 0.03%, 0.035%, 0.04%, 0.05%, 0.06%, 0.07%, 0.08%, 0.09%, 0.1%, 0.15%, 0.2%, 0.3%, 0.4%, 0.5%, 0.6%, 0.7%, 0.8%, 0.9%, 1% 1.5%, 2%, 2.5%, 3%, 3.5%, 4%, 4.5%, 5%, 6%, 7%, 8%, 9%, 10%, by weight, such as between at or about 0% and at or about 1%, by weight, of the composition, e.g. between at or about 0% and 1%, e.g. between at or about 0% and 0.03%, e.g. between at or about 0% and 0.025%, such as at or about 0.025%, by weight of the composition.

Other exemplary active ingredients include, but are not limited to nutritional supplements, phytochemicals, herbs, hormones, trace elements, electrolytes and minerals,

for example, compounds comprising aluminum, arsenic, boron, bromine, calcium, boron, chlorine, chromium, cobalt, copper, fluorine, fluoride, germanium, iodine, iron, lithium, magnesium, manganese, molybdenum, nickel, phosphorus, potassium, selenium, sodium, sulfur, silicon, tin, vanadium, zinc, metal salts, chelated minerals, colloidal minerals, colloidal silver, colloidal gold, bentonite;

neutraceuticals, pharmaceutical compositions, such as, but not limited to, anticonvulsants, analgesics (e.g. aspirin, acetaminophen, and others), antiparkinsons, anti-inflammatories, calcium antagonists, anesthetics, antimicrobials, antimalarials, antiparasitics, antihypertensives, antihistamines, antipyretics, alpha-adrenergic agonists, alpha-blockers, biocides, bactericides, bronchial dilators, beta-adrenergic blocking drugs, contraceptives, cardiovascular drugs, calcium channel inhibitors, depressants, diagnostics, diuretics, electrolytes, enzymes, hypnotics, hormones, hypoglycemics, hyperglycemics, muscle contractants, muscle relaxants, neoplastics, glycoproteins, nucleoproteins, lipoproteins, ophthalmics, psychic energizers, sedatives, steroids, sympathomimetics, parasympathomimetics, tranquilizers, urinary tract drugs, probiotics, vaccines, vaginal drugs, nonsteroidal anti-inflammatory drugs, angiotensin converting enzymes, polynucleotides, polypeptides, polysaccharides, natural and synthetic forms of immunosuppressive drugs, such as Cyclosporin, protease inhibitors such as Ritonavir, macrolide antibiotics, oil soluble anesthetics such as Propofol, natural and synthetic forms of steroidal hormones, for example, estrogens, estradiols, progesterone, testosterone, cortisone, phytoestrogens, dehydroepiandrosterone (DHEA), growth hormones and other hormones, and other drugs;

vitamins, in natural or synthetic form, including fat-soluble vitamins, for example, Vitamins A, D, E and K, and corresponding provitamins and vitamin derivatives such as, but not limited to, esters with an action resembling that of vitamin A, D, E or K for example; retinol (vitamin A) and pharmaceutically acceptable derivatives thereof, for example, palmitate ester of retinol and other esters of retinol, and calciferol (vitamin D) and its pharmaceutically acceptable derivatives thereof (e.g., ergosterol, ergocalciferol, cholecalciferol) and precursors of vitamin D, d-alpha tocopherol (vitamin E) and derivatives thereof, including pharmaceutical derivatives thereof, for example, Tocotrienols, d-alpha tocopherol acetate and other esters of d-alpha tocopherol, and ascorbyl palmitate, a fat-soluble version of vitamin C, and water-soluble vitamins, such as B vitamins, including Vitamin B1 (thiamine), Vitamin B2 (riboflavin), Vitamin B3 (niacin), Vitamin B5 (pantothenic acid), Vitamin B6 (pyridoxine), Vitamin B7 (biotin), Vitamin B9 (Folic acid) and Vitamin B12 (Cyanocobalamin), and Vitamin C (e.g., ascorbic acid, etc.);

other micronutrients, for example, co-factors, for example, coenzymes, such as, but not limited to, coenzyme Q, e.g. Coenzyme Q10 (CoQ10, also called ubiquinone, e.g. ubidecarenone or a reduced form of CoQ10, e.g. ubiquinol), tumeric extract (cucuminoids), saw palmetto lipid extract (saw palmetto oil), exhinacea extract, hawthorne berry extract, ginseng extract, alpha lipoic acid (thiotic acid), uric acid, acsorbyl palmitate, choline, kava extract, St. John's Wort (hyperium, Klamath weed, goat weed), extract of quercitin, dehydroepiandrosterone, indol-3-carbinol, 7-oxo-dehydroepiandrosterone, alpha-lipoic acid, betaine and betaine hydrochloride, CDP-choline (citicolin sodium), NADH, pantethine, pyruvate, S-adenosyl-L-methione (SAMe);

botantical extracts such as, but not limited to, DHEA, Ginkgo biloba extracts, ginseng extracts, reisi (Ganoderma) extract;

amino acids and oligopeptides, such as, but not limited to, 5-hydroxytryptophan, acetyl-L-carnitine, acetylcysteine, arginine pyroglutamate, branched-chain amino acids, creatine, DL-phenylalanine (phenylalanine), dimethylglycine (DMG), glutamine peptides, glutathione, glycine, insulin-like growth factor 1, L-arginine (arginine), L-aspartate, L-carnitine, L-cysteine, L-glutamine, L-histidine, L-lysine (lysine), L-methionine (methionine), L-ornithine, L-phenylalanine (phenylalanine), L-theanine, L-tyrosine (tyrosine), lactoferrin, ornithine alpha-ketoglutarate, para-aminobenzoic acid (aminobenzoic acid), L-taurine (taurine);

inosine, nucleic acids, nucleotides;

proteins such as, but not limited to, bovine cartilage, bovine colostrum, bromelain (bromelains), chicken collagen II, gelatin hydrolysates (gelatin), hydrolyzed collagen, shark cartilage, soy protein, whey proteins;

glycosupplements, such as, but not limited to, chitosan, chondroitin sulfate, D-glucarate, D-ribose, fructo-oligosaccharides, glucomannan, glucosamine, insulins (inulin), lactulose, larch arabinogalactan, modified citrus pectin, pectin, psyllium (psyllium husk), sodium alginates, yeast beta-D-glucans;

microorganisms such as but limited to prebiotics, probiotics, synbiotics, yoghurt organisms;

mycosupplements such as but not limited to brewer's yeast, kombucha, myco-polysaccharides, red yeast rice;

hormones, such as but not limited to 19-norandrostenedione, androstenediol, androstanedione, beta-sitosterol, biochanin A, DHEA, glandulars, human growth hormone and secretagogues (somatropin), ipriflavone, melatonin, pregnenolone, soy isoflavones, tiratricol;

compounds containing carotenoids, such as, but not limited to, hydrocarbons and oxygenated, alcoholic derivatives of hydrocarbons, for example, beta carotene, mixed carotenoids complex, leutein, lycopene, Zeaxanthin, Cryptoxanthin, for example, beta-crytoxanthin, beta carotene, mixed carotenoids complex, astaxanthin, bixin, canthaxanthin, capsanthin, capsorubin, apo-carotenal, beta-12′-apo-carotenal, “Carotene” (mixture of alpha and beta-carotene), gamma carotene, ciolerythrin, zeaxanthin, esters of hydroxyl- or carboxyl-containing members thereof;

compounds promoting weight loss or appetite suppression, such as, but not limited to, chromium picolinate, proteins, such as whey protein, casein protein, amino acids, such as glutamine, L-glutamine, calcium leucine, isoleucine, valine, L-taurine (taurine) and others, soy protein, hormones, such as estrogen, DHEA, pregnenolone and melatonin; hemp seed, creatine, creatine monohydrate, lactoferrin, Hoodia sp. Extracts, Garcinia cambogia (garcinia) extracts, including hydroxycitic acid, Taraxavum officinalis extracts, Paulina cupana (guarana) extracts, kalium phosphate, calcium phosphate, calcium fluoride, guar gum, Dahlia root extract, St. John's wort, capsicum, evodiamine, chitosan, Cassia mimosoides extracts, Opuntia ficus indica extracts, phaseolamin;

stimulants, such as thermogenics, e.g. sympathomimetics, caffeine, bitter orange, capsicum, ginger, guarana, and others;

fatty acid-containing compositions, such as essential fatty acid-containing compounds, e.g. polyunsaturated fatty acids (PUFA), including, for example, omega-3 fatty acids, for example, natural and synthetic omega-3 fatty acids, for example, compounds containing omega-3 polyunsaturated long-chain fatty acids, including Eicosapentaenoic acid (EPA) (20:5ω3); Docosahexaenoic acid (DHA) (22:6ω3); Eicosatetraenoic acid (24:4ω3); Docosapentaenoic acid (DPA, Clupanodonic acid) (22:5ω3); 16:3 ω3; 24:5 ω3 and/or nisinic acid (24:6ω3), for example, fish oil, algae oil, krill oil, canola oil, flaxseed oil, soybean oil and walnut oil; compounds containing short-chain omega-3 fatty acids, for example, Alpha-Linolenic acid (α-Linolenic acid; ALA) (18:3ω3) (e.g. flaxseed oil) and Stearidonic acid (18:4ω3), esters of an omega-3 fatty acid and glycerol, for example, monoglycerides, diglycerides and triglycerides, esters of omega-3 fatty acid and a primary alcohol, for example, fatty acid methyl esters and fatty acid esters, precursors of omega-3 fatty acid oils, for example, EPA precursor, DHA precursor, derivatives such as polyglycolized derivatives or polyoxyethylene derivatives, oils containing the omega-3 fatty acids, for example, fish oil (marine oil), for example, highly purified fish oil concentrates, perilla oil, krill oil, and algae oil, for example, microalgae oil; compounds containing omega 6 fatty acids, for example, compounds containing Linoleic acid (18:2ω6) (a short-chain fatty acid); Gamma-linolenic acid (GLA) (18:3ω6); Dihomo gamma linolenic acid (DGLA) (20:3ω6); Eicosadienoic acid (20:2ω6); Arachidonic acid (AA) (20:4ω6); Docosadienoic acid (22:2ω6); Adrenic acid (22:4ω6); and/or Docosapentaenoic acid (22:5ω6), for example, borage oil, corn oil, cottonseed oil, grapeseed oil, peanut oil, primrose oil, for example, evening primrose (Oenothera biennis) oil, blackcurrant seed oil, hemp seed oil, spurulina extract, safflower oil, sesame oil and soybean oil;

omega-9 fatty acids, e.g. Oleic acid (18:1 ω9); Eicosenoic acid (20:1 ω9); Mead acid (20:3 ω9); Erucic acid (22:1 ω9); and Nervonic acid (24:1 ω9);

conjugated fatty acids, such as conjugated linoleic acid (CLA), for example, 18:2 ω7, 18:2 ω6; Conjugated Linolenic acid, for example, 18:3ω6, 18:3ω5; and other conjugated fatty acids, for example, 18:3 ω3, 18:4 ω3, and 20:5 ω6;

other fatty acids, for example, triglycerides, including medium chain triglycerides, polar lipids, for example, ether lipids, phosphoric acid, choline, fatty acids, glycerol, glycolipids, triglycerides, and phospholipids (e.g., phosphatidylcholine (lecithin), phosphatidylethanolamine, and phosphatidylinositol); saw palmetto extract; and ethyl linoleate; and herb oils, for example, garlic oils and scordinin; short-chain saturated fatty acids (4:0-10:0), Lauric acid (12:0), Myristic acid (14:0), Pentadecanoic acid (15:0), Palmitic acid (16:0), Palmitoleic acid (16:1 ω7), Heptadecanoic acid (17:0), Stearic acid (18:0), Oleic acid (18:1 ω9), Arachidic acid (20:0);

phytochemicals, including phytoestrogens, for example, lignan, genistein and daidzein, for example, isoflavones, for example, soy isoflavones, apigenin, luteolin, tangeritin, flavonoids, phytoalexins, for example, Resveratrol (3,5,4′-trihydroxystilbene), red clover extract, and phytosterols;

acids and alcohols, for example, tartaric acid, lactylic acid, butylated hydroxyanisole, butylated hydroxytoluene, lignin, sterols, polyphenolic compounds, oryzanol, cholesterol, phytosterols, flavonoids, including flavones, flavonols, such as quercetin, flavanones, isoflavone phytoestrogens, stilbenoids, such as reservatol, anthocyanins, diallyl disulfides;

other supplements known in the art such as but not limited to activated charcoal, beta-hydroxy-beta-methylbutyrate (HMB), choline, colosolic acid, deanol, dimethyl sulfoxide (DMSO), dolomite, gamma-butyrolactone (GBL), gamma-hydroxybutyrate (GHB), liver hydrolysate/desiccated liver, malic acid, methylsulfonylmethane (MSM), royal jelly, vinpocetine, arnica, bee pollen, chlorella, chlorophyll/chlorophyllin (chlorophyllin copper complex), chrysin, cocoa flavonoids, curcuminoids, daidzein, deglycyrrhizinated licorice (DGL), flower pollen, genistein, glycitein, grape seed proanthocyanidins, green tea catechins, black tea theaflavins, hesperetin, hesperidin, huperzine A, hydroxycitric acid, hydroxyethylrutosides, indole-3-carbinol, lutein and zeaxanthin, lycopene, oat beta-D-glucan, phytostanols, phytosterols, piperine, propolis, pycnogenol, quercetin, resveratrol, rutin, secoisolariciresinol diglycoside (SDG), soy isoflavones, spirulina, sulforaphane, wheat grass/barley grass and others.

In one example, the composition contains CLA. The amount of CLA in the composition can be between at or about 0% and at or about 50%, by weight, of the composition, and typically is between at or about 0% and at or about 25%, and typically between at or about 0% and at or about 10% or between at or about 0% and at or about 5%, e.g. at or about 1%, 2%, 3%, 4% or 5%, or between at or about 0% and 1%, such as at or about 0%, 0.01%, 0.015%, 0.02%, 0.025%, 0.03%, 0.035%, 0.04%, 0.05%, 0.06%, 0.07%, 0.08%, 0.09%, 0.1%, 0.15%, 0.2%, 0.21%, 0.22%, 0.23%, 0.24%, 0.241%, 0.242%, 0.243%, 0.244%, 0.245%, 0.246%, 0.247%, 0.2475%, 0.248%, 0.249%, 0.25%, 0.26%, 0.27%, 0.271%, 0.272%, 0.273%, 0.274%, 0.275%, 0.3%, 0.4%, 0.5%, 0.6%, 0.7%, 0.8%, 0.9%, 1% 1.5%, 2%, 2.5%, 3%, 3.5%, 4%, 4.5%, 5%, 6%, 7%, 8%, 9%, 10%, such as between at or about 0% and at or about 1%, by weight, of the composition, e.g. between at or about 0% and 1%, e.g. between at or about 0% and 0.03%, e.g. between at or about 0% and 0.025%, such as at or about 0.025%, by weight, of the composition.

2. Taste-Modifying Agents

The provided compositions typically contain one or more taste-modifying agents, such as taste-modifying agents to render the compositions palatable, e.g. having pleasant (or not unpleasant) taste and/or smell, such that they are desirable for consumption by a subject, e.g. a human, such as by oral ingestion. Taste-modifying agents in the provided compositions include, but are not limited to, sweetening agents and flavoring agents, e.g. compounds that confer flavors to compositions (e.g. fruit and other flavors) and taste/flavor masking agents. The compositions typically contain a combination of taste-modifying agents, such as a combination of a flavoring agent and sweetening agent, at an amount sufficient to make the composition palatable.

The taste-modifying agents include flavoring agents, such as, but not limited to, flavoring agents that confer fruit flavors, for example, peach, guava, kiwi, mango, papaya, pineapple, grape, banana, strawberry, raspberry, blueberry, orange, grapefruit, tangerine, lemon, lime, cherry, apple, plum, watermelon, coconut, and other fruit flavors, cola flavors, tea flavors, coffee flavors, chocolate flavors, dairy flavors, root beer and birch beer flavors, methyl salicylate (wintergreen oil, sweet birch oil), citrus oils (e.g. lemon oil), essential oils or water soluble extracts of menthol, wintergreen, peppermint, mint, sweet mint, spearmint, natural and artificial vanilla, nut flavor, nuts, chocolate, fudge, butterscotch, cinnamon, clove, rose, spice, violet, herbal, durean, green tea, butter, cream custard, chamomile; and sweetening agents, such as sugar or sugar substitutes, such as sucralose, dextrose, lactose, mannitol, sucrose, xylitol, malitol, acesulfame potassium, talin, glycyrrhizin, aspartame, saccharin, sodium saccharin, neotame, sodium cyclamate and honey. For example, provided are compositions containing taste-modifying agents that include a flavoring agent, such as a flavoring agent that confers a fruit flavor (e.g. peach or sour apple), a mint flavor (e.g. spearmint or peppermint) and/or a cinnamon flavor, and a sugar substitute (e.g. sucralose).

Typically, the taste-modifying agents contain a flavoring agent that confers a fruit flavor, typically a peach flavor, e.g. peach flavoring agent sold by Mission Flavors and Fragrances, Inc., Foothill Ranch, Calif., (Catalog number PH-147), a spearmint flavor, such as the spearmint flavoring agent sold by Mission Flavors and Fragrances, Inc. (Catalog number MI-110), a mint flavoring agent sold by Mission Flavors and Fragrances, Inc. (Catalog number L-12389), a peppermint flavor, such as the peppermint flavoring agent sold by Mission Flavors and Fragrances, Inc. (Catalog number MI-104), a cinnamon flavor, such as the cinnamon flavoring agent sold by Mission Flavors and Fragrances, Inc. (Catalog number L-9806), and/or a sour apple flavor, such as the sour apple flavoring agent sold by Mission Flavors and Fragrances, Inc. (Catalog number AP-138), and a sugar substitute, typically sucralose. Exemplary of the sucralose includes Trichlorosucrose (sucralose) sold by CHANGZHOU TIANHUA I&E CO LTD, NIUTANG TOWN, CHANGZHOU JIANGSU CHINA, or any other sucralose.

The amount of the taste-modifying agents (e.g. flavors and sweeteners) typically is sufficient to make the compositions palatable or to improve the palatability of the compositions, compared to the composition in the absence of the taste-modifying agents. Sufficient amounts of each taste-modifying agent and of the combination of taste-modifying agents, as well as the appropriate choice of taste-modifying agents and combinations thereof, can be determined empirically, e.g. by testing various amounts of the taste-modifying agents and evaluating palatability, or can be determined based on the formulation of a previously made compositions, such as exemplary compositions provided herein.

The amount of individual taste-modifying agents (e.g. the amount of flavoring agent or sweetener, such as sugar substitute), and/or the combination of taste-modifying agents (e.g. the total amount of taste-modifying agents) in the composition can be between at or about 0% and at or about 50%, by weight, of the composition, and typically is between at or about 0.01% and at or about 25%, and typically is between at or about 0.01% and at or about 10% or between at or about 0.01% and at or about 5%, e.g. at or about 0.01%, 0.015%, 0.02%, 0.025%, 0.03%, 0.035%, 0.04%, 0.05%, 0.06%, 0.07%, 0.08%, 0.09%, 0.1%, 0.15%, 0.2%, 0.3%, 0.4%, 0.5%, 0.6%, 0.7%, 0.8%, 0.9%, 1% 1.5%, 2%, 2.5%, 3%, 3.5%, 4%, 4.5%, 5%, 6%, 7%, 8%, 9%, 10%, such as between at or about 0.1% and at or about 25%, 10%, 5% or 1%, by weight, of the composition, e.g. between at or about 0.1% and 2%, such as at or about 0.1% 0.11%, 0.12%, 0.13%, 0.14%, 0.15%, 0.16%, 0.17%, 0.18%, 0.19%, 0.2%, 0.3%, 0.4%, 0.45%, 0.5%, 0.6%. 0.7%, 0.8%, 0.9%, 1.0%, 1.09%, 1.1%, 1.2%, 1.3%, 1.4%, 1.5%, 1.6%, by weight, e.g. between at or about 0.45% and at or about 1.5%, such as at or about 0.45%, 0.5%, 1%, 1.09%, 1.2%, 1.4%, 1.43%, 1.438%, 1.45% or 1.5%, by weight of the composition. In one example, the amount of taste-modifying agent (e.g. flavoring agent and/or sugar substitute) is less than at or about 10%, by weight, of the composition, or less than at or about 5%, by weight, of the composition, such as between at or about 0.1% and at or about 5%, between at or about 0.45% and at or about 3%, between at or about 0.1% and at or about 1.5%, between at or about 2% and at or about 3%, by weight of the composition, e.g. at or about 0.45%, 1.5%, 1%, 1.438%, 1.5%, 1.88%, 2.94%, 2% or 3%, by weight, of the composition.

3. Surfactants and Co-Surfactants

The provided compositions can further include surfactants, typically TPGS surfactants, TPGS analogs, and other surfactants having similar properties, such as similar HLB values; and co-surfactants, such as phospholipid-containing compounds, such as phosphatidylcholine.

Surfactants are well known, and include, for example, polyethylene glycol (PEG)-derived surfactants, such as PEG-derivatives of Vitamin E and PEG-fatty acid esters, including esters of lauric acid, oleic acid, and stearic acid, sorbitan monolaurate (Span 20), sorbitan monopalmitate (Span 40), sorbitan monostearate (Span 60), sorbitan monooleate (Span 80), polyoxyethylene (20) sorbitan monolaurate (Tween 20, polysorbate 20), polyoxyethylene (20) monopalmitate (Tween 40, polysorbate 40), polyoxyethylene (20) monostearate (Tween 60, polysorbate 60), polyoxyethylene (20) tri-stearate (Tween 65, polysorbate 65), polyoxyethylene (20) monooleate (Tween 80, polysorbate 80), sucrose monomyristate, sucrose palmitate/stearate, sucrose stearate, dioctylsulfosuccinate sodium salt (DOSS), monoglyceride monooleate, monoglyceride monolaurate, monoglyceride monopalmitate, lecithin, diglyceride mixtures, citric acid esters of monoglycerides, acetic acid esters of monoglycerides, lactic acid esters of monoglycerides, diacetyl tartaric esters of monoglycerides, polyglycerol esters of fatty acids such as decaglycerol monocaprylate/caprate, triglyceryl monooleate, decaglycerol monostearate, decaglycerol dipalmitate, decaglycerol monooleate, decaglycerol tetraoleate and hexaglycerol dioleate, cyclodextrins (α, β, or γ), propylene glycol esters of fatty acids such as dicaprate esters, mono and dicaprylate ester blends and diesters of caprylate and capric acids, stearoyl lactylates and free fatty acids, and any known surfactants, such as, but not limited to, any of the surfactants listed in Table 1, above and any of the surfactants described in U.S. Pat. No. 6,267,985.

Typically, the surfactants include Vitamin E-derived surfactants (e.g. a tocopherol-derived or a tocotrienol-derived surfactant), and similar surfactants, e.g. surfactants having a similar HLB value (e.g. surfactants having an HLB (hydrophilic-lipophilic balance) value of between 14 or about 14 and 20 or about 20, for example, 14, 15, 16, 17, 18, 19, 20, about 14, about 15, about 16, about 17, about 18, about 19 or about 20), such as polysorbate 80 and other PEG-derived surfactants, such as PEG-sorbitan fatty acid esters, such as polysorbates, including polysorbate 80 and analogs (e.g. homologs) of polysorbate 80, such as, for example, polysorbate 20, polysorbate 40 and polysorbate 60, and other polysorbates.

HLB is a value, derived from a semi-empirical formula, which is used to index surfactants according to their relative hydrophobicity/hydrophilicity. An HLB value is a numerical representation of the relative representation of hydrophilic groups and hydrophobic groups in a surfactant or mixture of surfactants. The weight percent of these respective groups indicates properties of the molecular structure. See, for example, Griffin, W. C. J. Soc. Cos. Chem. 1:311 (1949).

Surfactant HLB values range from 1-45, while the range for non-ionic surfactants typically is from 1-20. The more lipophilic a surfactant is, the lower its HLB value. Conversely, the more hydrophilic a surfactant is, the higher its HLB value. Lipophilic surfactants have greater solubility in oil and lipophilic substances, while hydrophilic surfactants dissolve more easily in aqueous liquids. In general, surfactants with HLB values greater than 10 or greater than about 10 are called “hydrophilic surfactants,” while surfactants having HLB values less than 10 or less than about 10 are referred to as “hydrophobic surfactants.” HLB values are known for a number of surfactants Table 1 lists HLB values of exemplary surfactants and co-surfactants.

Exemplary of the Vitamin E-derived surfactants are polyalkylene glycol derivatives, typically polyethylene glycol (PEG) derivatives, of Vitamin E, for example, PEG derivatives of tocopherol, such as, but not limited to, TPGS and analogs thereof. Suitable PEG derivatives of Vitamin E typically contain one or more tocopherols or tocotrienols, joined (for example, by an ester, ether, amide or thioester bond) with one or more PEG moieties, via a linker, for example, a dicarboxylic acid linker. An exemplary surfactant is shown schematically below:

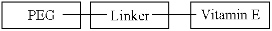

where the line between the PEG and Linker; and the line between the Linker and Vitamin E each independently represent a covalent bond selected from among an ester, ether, amide or thioester.

Typically, the Vitamin E PEG derivatives are made by joining the PEG moiety, via esterification, to a vitamin E-linker conjugate (e.g. a tocopherol-linker conjugate). In one example, the tocopherol-linker conjugate first is formed by covalently joining (by esterification) the hydroxyl moiety of tocopherol with a dicarboxylic acid to produce an ester bond. In this example, the tocopherol-linker conjugate is a tocopherol ester (such as tocopherol succinate). The esterification reaction can be performed by any of a number of known methods (see, for example, U.S. Pat. Nos. 2,680,749, 4,665,204, 3,538,119 and 6,632,443). To make the tocopherol-PEG surfactant, the resulting tocopherol ester then is joined (via the linker) to the PEG molecule, in another esterification reaction. In this example, the resulting surfactant is a tocopherol polyethylene glycol diester (TPGD).

Alternatively, PEG derivatives of a tocopherol-linker or tocotrienol-linker conjugate can be made by other methods. Various methods known in the art for producing PEG derivatives can be used to join a PEG molecule to tocopherol-linker or tocotrienol-linker compounds. For example, a tocopherol-linker conjugate can be covalently bonded to the PEG molecule via an amide, ether or thioether bond. For example, a tocopherol-linker conjugate that contains an amine group can be reacted with a PEG-NHS derivative to form an amide bond between the tocopherol-linker and the PEG molecule. A tocopherol-linker conjugate that contains an amine group can be reacted with a PEG-aldehyde derivative to form an amide bond between the tocopherol-linker and the PEG molecule. In another example, a tocopherol-linker that contains an carboxylic acid can be activated to the corresponding acid halide and reacted with a PEG-SH derivative to form a thioester bond between the tocopherol-linker and the PEG molecule.

The tocopherol(s) used to make the Vitamin-E derived surfactant can be any natural or synthetic Vitamin E tocopherol, including but not limited to alpha-tocopherols, beta-tocopherols, gamma-tocopherols and delta tocopherols, either in pure forms or in heterogenous mixtures of more than one form. Exemplary tocopherols are d-α tocopherols and d, 1-tocopherols. To make the surfactant, the tocopherol typically is esterified with a linker, for example, a dicarboxylic acid, to form a tocopherol ester, which then is joined to a PEG moiety.

The tocotrienol(s) used to make the surfactants can be any natural or synthetic Vitamin E tocotrienol, including but not limited to alpha-tocotrienols, beta-tocotrienols, gamma-trienols and delta tocotrienols, either in pure forms or in heterogenous mixtures of more than one form. Mixtures of tocopherols and tocotrienols, are contemplated for use in the provided methods and compositions. A tocotrienol can be esterified with a linker, such as a dicarboxylic acid, before joining with a PEG moiety.

Typically, the Vitamin E PEG-derivative surfactants are diesters or other esters, e.g. triesters. When the PEG derivative is a diester, the linker joining the Vitamin E to the PEG typically is a carboxylic acid, typically a dicarboxylic acid, as in, for example, tocopherol polyethylene glycol succinate (TPGS), where the linker is a succinic acid, and the surfactant is made by an esterification reaction joining a PEG moiety and a tocopherol ester of the dicarboxylic acid. In another example, the linker is another molecule, for example, an amino acid, such as glycine, alanine, 5-aminopentanoic acid or 8-aminooctanoic acid; or an amino alcohol, such as ethanol amine.

Typically, the surfactants are vitamin E polyethylene glycol diesters, which are Vitamin E esters of PEG, made by joining a Vitamin E ester to one or more PEG moieties by esterification. Exemplary of the Vitamin E diesters are tocopherol polyethylene glycol diesters (TPGD) and tocotrienol polyethylene glycol diesters.

When the tocopherol or tocotrienol ester linked with the PEG moiety is a tocopherol ester of a dicarboxylic acid (e.g. tocopherol succinate), the linker is a dicarboxylic acid (a carboxylic acid having two carboxy groups, e.g. succinic acid). In this example, the tocopherol or tocotrienol PEG diester is formed by esterification reaction, in which PEG is attached to a tocopherol ester of a dicarboxylic acid.

Exemplary of dicarboxylic acids that can be used as linkers in these tocopherol and tocotrienol PEG diester surfactants are succinic acid, sebacic acid, dodecanodioic acid, suberic acid, azelaic acid, citraconic acid, methylcitraconic acid, itaconic acid, maleic acid, glutaric acid, glutaconic acid, fumaric acids and phthalic acids. Accordingly, exemplary of the tocopherol esters that can be esterified to form the PEG-derivatives are tocopherol succinate, tocopherol sebacate, tocopherol dodecanedioate, tocopherol suberate, tocopherol azelaate, tocopherol citraconate, tocopherol methylcitraconate, tocopherol itaconate, tocopherol maleate, tocopherol glutarate, tocopherol glutaconate, and tocopherol phthalate, among others.

Exemplary of the vitamin E polyethylene glycol diesters made with dicarboxylic acids are compounds having the following formula shown in scheme I below (and homologs, analogs and derivatives thereof):

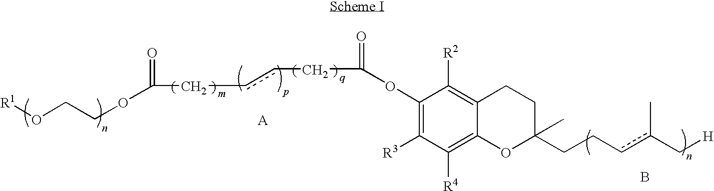

where R¹, R², R³ and R⁴ each independently is H or Me; each dashed line is independently a single or double bond; n is an integer from 1-5000; m and q each independently are 0 or 1; and p is an integer from 1-20. In one example, the surfactant is a compound where, when both m and q are 0, p is an integer between 2-20.

In one example, the surfactant has the following formula shown in Scheme II below (including homologs, analogs and derivatives thereof):

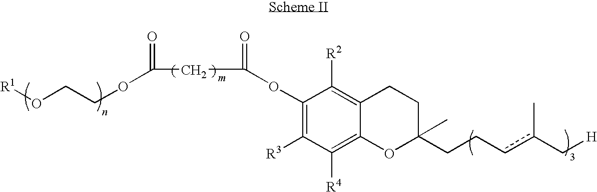

where when R1, R2, R3 and R4 represent a hydrogen or methyl, the bond represented by the dashed line is either a single or double bond, m is any integer between 1 and 20, and n=1-5000.

Exemplary of tocopherol and tocotrienol PEG diesters that can be used as surfactants in the provided compositions and methods include, but are not limited to: tocopherol polyethylene glycol succinates (TPGS; including d-α TPGS and d,1-TPGS; see for example, U.S. Pat. No. 3,102,078), tocopherol polyethylene glycol sebacate (PTS; see for example, U.S. Pat. No. 6,632,443), tocopherol polyethylene glycol dodecanodioate (PTD; see for example, U.S. Pat. No. 6,632,443), tocopherol polyethylene glycol suberate (PTSr; see for example, U.S. Pat. No. 6,632,443), tocopherol polyethylene glycol azelaate (PTAZ; see for example, U.S. Pat. No. 6,632,443), polyoxyethanyl tocotrienyl sebacate (PTrienS, for example, PTrienS-600; see for example, U.S. Pat. No. 6,632,443), as well as analogs, homologs and derivatives or any of the tocopherol diesters.

In another example, the tocopherol ester joined to the PEG to form the tocopherol PEG diester is a tocopherol ester of a tricarboxylic acid, for example, Citric acid, Isocitric acid, Aconitic acid and Propane-1,2,3-tricarboxylic acid (tricarballylic acid, carballylic acid), or a carboxylic acid having three or more carboxy groups.

In another example, the PEG derivatives of tocopherol are tocopherol polyethylene glycol triesters (TPGT), for example, esters containing a tocopherol, a linker, a PEG moiety, and an additional moiety, for example, an additional tocopherol, a second PEG moiety, or a water-soluble group, such as a quaternary amine. In one example, when the triester contains two PEG moieties, each PEG moiety has a smaller chain length (and lower molecular weight) than the PEG moiety in a PEG derivative of tocopherol, having similar properties, that contains only one PEG chain.

The PEG moieties used in the tocopherol-PEG derivative surfactants or other PEG-derivative surfactants include any of a plurality of known PEG moieties. Exemplary of suitable PEG moieties are PEG moieties having varying chain lengths, and varying molecular weights, for example, PEG 1000, PEG 200, PEG 500, and PEG 20,000. The numbers following individual PEG moieties indicate the molecular weight (in kilodaltons (KDa) of the PEG moieties. The PEG moiety of the surfactant typically has a molecular weight of between 200 or about 200 to 20,000 or about 20,000 KDa, typically between 200 and 6000 KDa, for example, between 600 or about 600 KDa and 6000 or about 6000 KDa, typically between 200 or about 200 KDa and 2000 or about 2000 KDa, between 600 or about 600 KDa and 1500 or about 1500 KD, or at or about 200, 300, 400, 500, 600, 800, and 1000 KDa. Exemplary of a PEG-derivative of tocopherol ester having a PEG moiety with 1000 KDa is TPGS-1000. Also exemplary of suitable PEG moieties are PEG moieties that are modified, for example, methylated PEG (m-PEG), which is a PEG chain capped with a methyl group. Other known PEG analogs also can be used. The PEG moieties can be selected from among any reactive PEG, including, but not limited to, PEG-OH, PEG-NHS, PEG-aldehyde, PEG-SH, PEG-NH₂, PEG-CO₂H, and branched PEGs.

a. TPGS Surfactants

Typically, the surfactants are polyethylene glycol diester surfactants such as TPGS, and analogs, homologs and derivatives thereof, including vitamin E PEG diesters, such as, but not limited to, tocophyrol polyethylene glycol sebacate (PTS), tocopherol polyethylene glycol dodecanodioate (PTD), tocopherol polyethylene glycol suberate (PTSr), tocopherol polyethylene glycol azelaate (PTAz) and polyoxyethanyl tocotrienyl sebacate (PTrienS) as well as other PEG derivatives of Vitamin E.

TPGS is a natural surfactant that is GRAS and Kosher certified and thus, desirable for use in products designated for human consumptions, for example, beverages, food and nutritional supplements. TPGS typically has an HLB value of between 16 or about 16 and 18 or about 18. Exemplary of the TPGS surfactants is TPGS-1000, which has a PEG moiety of 1000 KDa. Exemplary of the TPGS surfactants that can be used in the provided compositions is the food grade TPGS surfactant sold under the name Eastman Vitamin E TPGS®, food grade, by Eastman Chemical Company, Kingsport, Tenn. This surfactant is a water-soluble form of natural-source vitamin E, which is prepared by esterifying the carboxyl group of crystalline d-alpha-tocopheryl acid succinate with polyethylene glycol 1000 (PEG 1000), and contains between 260 and 300 mg/g total tocopherol.

A similar compound can be made by esterifying the carboxyl group of the d,1 form of synthetic Vitamin E with PEG 1000. It forms a clear liquid when dissolved 20% in water. This tocopheryl polyethylene glycol is a water-soluble preparation of a fat-soluble vitamin (vitamin E), for example, as disclosed in U.S. Pat. Nos. 3,102,078 and 2,680,749 and U.S. Published Application Nos. 2007/0184117 and 2007/0141203. The PEG moiety of alternative TPGS surfactants can have a molecular weight range of 200 or about 200 to 20,000 or about 20,000 KDa, for example, between 600 or about 600 KDa and 6000 or about 6000 KDa, typically between 600 or about 600 KDa and 1500 or about 1500 KDa. Also exemplary of the TPGS surfactant that can be used in the provided compositions is the Water Soluble Natural Vitamin E (TPGS), sold by ZMC-USA, The Woodlands, Tex. Any known source of TPGS, or any analog, homolog or derivative thereof, can be used.

At room temperature, TPGS typically is a waxy low-melting solid. In one example, the TPGS is heated prior to use, for example, to at least the melting temperature, for example, between 37° C. or about 37° C. and 41° C. or about 41° C. and the desired amount is poured out. In another example, the TPGS can be added as a waxy solid to a vessel and heated with the heating apparatus.

The amount of surfactant, e.g. the TPGS or other Vitamin E derived surfactant in the composition can be between at or about 0% and 50%, by weight, and typically is between at or about 0.1% and at or about 25%, such as at or about 0.1%, 0.2%, 0.3%, 0.4%, 0.5%, 0.6%, 0.61%, 0.62%, 0.625%, 0.63%, 0.64%, 0.65%, 0.66%, 0.67%, 0.68%, 0.69%, 0.7%, 0.71%, 0.72%, 0.73%, 0.74%, 0.75%, 0.76%, 0.77%, 0.78%, 0.79%, 0.8%, 0.9%, 1.0%, 1.1%, 1.2%, 1.25%, 1.3%, 1.35%, 1.4%, 1.45% or 1.5%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24% or 25%, such as between at or about 0.1% and at or about 5%, or between at or about 0.1% and at or about 2%, or between at or about 0.6% and at or about 1.3%, e.g. between at or about 0.625% and at or about 1.25%. In one example, the amount of surfactant is less than 5%, by weight, of the composition, e.g. between at or about 0.01% and 5% or between at or about 0.01% and 1.25%, e.g. at or about 1.25%, 1.15%, 1%, 0.9%, 0.8%, 0.75%, 0.7%, 0.675%, 0.65%, 0.625%, e.g. between at or about 0.625% and at or about 0.75% or between at or about 0.625% and at or about 1.25%, by weight, of the composition.

b. Co-surfactants

The provided compositions can further contain co-surfactants (emulsifiers). The co-surfactants can include phospholipids, such as, but not limited to, phosphatidylcholine. Phospholipids are amphipathic lipid-like molecules, typically containing a hydrophobic portion at one end of the molecule and a hydrophilic portion at the other end of the molecule. A number of phospholipids can be used as co-surfactants in the provided compositions, for example, lecithin, including phosphatidylcholine (PC), phosphatidylethanolamine (PE), distearoylphosphatidylcholine (DSPC), phosphatidylserine (PS), phosphatidylglycerol (PG), phosphatidic acid (PA), phosphatidylinositol (PI), sphingomyelin (SPM) or a combination thereof. Typically, the phospholipid is phosphatidylcholine (PC), which sometimes is referred to by the general name “lecithin.” Exemplary of the phospholipids that can be used as co-surfactants in the provided compositions are the phospholipids sold by Lipoid, LLC, Newark, N.J., for example, Purified Egg Lecithins, Purified Soybean Lecithins, Hydrogenated Egg and Soybean Lecithins, Egg Phospholipids, Soybean Phospholipids, Hydrogenated Egg and Soybean Phospholipids. Synthetic Phospholipids, PEG-ylated Phospholipids and phospholipid blends sold by Lipoid, LLC.

The phosphatidylcholine co-surfactants include, but are not limited to, the phosphatidylcholine composition sold by Lipoid, LLC, under the name Lipoid S-100 and the phosphatidylcholine composition sold by Lipoid, LLC, under the name Alcolec PC95, each of which is derived from soy extract and contains greater than 95% or greater than about 95% phosphatidylcholine.

The amount of co-surfactant in the composition can be between at or about 0% and at or about 50%, by weight, and typically is between at or about 0% and at or about 25%, by weight, e.g. at or about 0.01%, 0.02%, 0.03%, 0.04%, 0.05%, 0.06%, 0.07%, 0.075%, 0.08%, 0.085%, 0.09%, 0.095%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24% or 25%, such as between at or about 0% and 1%, or between at or about 0% and 0.1%, e.g. between at or about 0% and 0.075%, such as at or about 0.075%. In one example, the amount of phospholipid (e.g. phosphatidylcholine) is less than 1% or less than 0.1%, by weight, of the composition, and typically is less than the amount of the surfactant.

4. Water

The compositions typically are aqueous liquids and further include water, typically purified water. In one example, the water is purified prior to adding it to the composition, for example, by charcoal filter, ion exchange, reverse osmosis, UV sterilization and/or filtering using a filter, for example, a 50-100 micron filter. Typically, when a filter is used, it is an end point of use filter, which filters the water before it reaches the tank in the provided process. Alternatively, previously filtered water can be added to the compositions.

The amount of water in the composition can be between at or about 0% and at or about 99%, by weight or between at or about 0.01% and at or about 95%, and typically is between at or about 10% and at or about 95%, by weight, such as at or about 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 89.1%, 89.2%, 89.3%, 89.4%, 89.465%, 89.47%, 89.5%, 89.6%, 89.7%, 89.8%, 89.9%, 89.965%, 90%, 90.1%, 90.2%, 90.3%, 90.388%, 90.39%, 90.4%, 90.5%, 90.515%, 90.52%, 90.6%, 90.7%, 90.8%, 90.9%, 91%, 91.1%, 91.2%, 91.3%, 91.332%, 91.4%, 91.5%, 91.6%, 91.7%, 91.8%, 91.8568%, 91.86%, 91.9%, 92%, 93%, 94% or 95%, and in some examples is greater than at or about 70%, by weight, of the composition, such as between at or about 70% and at or about 95%, by weight, of the composition, e.g. between at or about 85% and at or about 95%, such as between at or about 89% and at or about 92%, by weight or between at or about 89% and at or about 91%, by weight. In some examples, the amount of water is an appropriate amount of water that conveys an aqueous composition. In some cases, the amount of water is selected according to the desired amounts (e.g. concentrations) of the other ingredients of the composition, for example, enough water to dissolve the desired amount of the aminoalkane or other active ingredient. In some examples, water is added to bring the composition up to the desired volume, e.g. low volume, such as between at or about 1 mL and at or about 10 mL, e.g. 1, 2, 3, 4, 5, 6, 7, 8, 9 or 10 mL. In such an example, the water can be added during the water phase production and/or after combining the water and oil phases of the composition, according to the provided methods for making the compositions.

5. Preservatives and Sterilizers

Typically, the provided compositions further contain preservatives and/or sterilizers, such as preservatives at an amount sufficient to stabilize the composition. Preservatives, particularly food and beverage preservatives, are well known. Any known preservative can be used in the provided compositions. Exemplary of the preservatives that can be used in the provided compositions are oil soluble preservatives, for example, potassium sorbate, sodium benzoate, benzyl alcohol, Benzyl Benzoate, Methyl Paraben, Propyl Paraben, antioxidants, for example, Vitamin E, Vitamin A Palmitate and Beta Carotene. Typically, a preservative is selected that is biocompatible, e.g. safe for human consumption, for example, in foods and beverages, for example, a GRAS certified and/or Kosher-certified preservative. In one example, the preservatives include sodium benzoate and potassium sorbate, which are commercially available.

The amount of individual preservatives (e.g. sodium benzoate, potassium sorbate or other preservative) and/or the combination of total preservatives, in the composition typically is an amount sufficient to preserve and/or stabilize the composition compared to composition in the absence of the preservative, and can be between at or about 0% and 50%, by weight, and typically is at or about 0.001% and at or about 25%, by weight, of the composition, e.g. at or about 0.001%, 0.002%, 0.003%, 0.004%, 0.005%, 0.006%, 0.007%, 0.008%, 0.009%, 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, such as between at or about 0.01% and at or about 10%, by weight of the composition, e.g. between at or about 0.01% and at or about 1% or at or about 0.1%, by weight, such as at or about 0.04% or at or about 0.08%, by weight. In one example, the compositions contain two preservatives, each at a concentration of at or about 0.04%, by weight, of the composition.

6. pH adjusting agents

In one example, the provided compositions further include one or more pH adjusting agents (pH adjusters). Typically, the pH adjuster is added to adjust the pH of the composition to within a range of 2.0 or about 2.0 to 4.0 or about 4.0, such as between at or about 2.0 and 2.4. One or more of a plurality of pH adjusting agents can be used. Typically, the pH adjusting agent is safe for human consumption, for example, GRAS certified. Exemplary of the pH adjuster is citric acid, for example, the citric acid sold by Mitsubishi Chemical, Dublin, Ohio.

The amount of pH adjusting agent typically is the amount of the pH adjusting agent sufficient to adjust the pH to the desired amount, e.g. to change (increase or decrease) the pH of the composition compared to the composition without the pH adjusting agent, for example, to render the pH of the composition between at or about 2 and at or about 5, e.g. 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, or 5, such as between at or about 2 and at or about 4, e.g. between at or about 2 and at or about 3.5 or between at or about 2 and at or about 3.3; the appropriate amount of the pH adjusting agent can be determined empirically, such as by adding different amounts of pH adjusting agent(s) and measuring pH of the composition, for example, as described below. The amount of the pH adjuster typically is between at or about 0% and at or about 10%, by weight of the composition, and typically between at or about 0% and at or about 5%, by weight, of the composition, such as, for example, at or about 0.01%, 0.02%, 0.03%, 0.04%, 0.05%, 0.06%, 0.07%, 0.08%, 0.09%, 0.1%, 0.11%, 0.12%, 0.13%, 0.14%, 0.15%, 0.16%, 0.17%, 0.18%, 0.19%, 0.2%, 0.3%, 0.4%, 0.5%, 0.6%, 0.7%, 0.8%, 0.9%, 2%, 2%, 2.1%, 2.2%, 2.3%, 2.4%, 2.5%, 2.6%, 2.65%, 2.655%, 2.66%, 2.7%, 2.8%, 2.9%, 3%, 4% or 5%, by weight, of the composition.

7. Emulsion Stabilizers

Typically, the provided compositions further contain one or more emulsion stabilizers (co-emulsifiers), which can stabilize the compositions. Exemplary of an emulsion stabilizer that can be used in the provided compositions is a composition containing a blend of gums, for example, gums used as emulsifying agents, for example, a blend containing one or more of xanthan gum, guar gum and sodium alginate, for example, the emulsion stabilizer sold under the brand name SALADIZER®, available from TIC Gums, Inc. (Belcamp, Md.). Other gums can be included in the emulsion stabilizer, for example, gum acacia and sugar beet pectin. Other blends of similar gums can also be used as emulsion stabilizers.

The amount of the emulsion stabilizer in the composition typically is an amount sufficient to stabilize the composition compared to the composition in the absence of the emulsion stabilizer, and can be determined empirically and can be between at or about 0% and at or about 50%, and typically between at or about 0% and at or about 25%, by weight, e.g. at or about 0.01%, 0.02%, 0.03%, 0.04%, 0.05%, 0.06%, 0.07%, 0.08%, 0.09%, 0.1%, 0.11%, 0.12%, 0.13%, 0.14%, 0.15%, 0.16%, 0.165%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, by weight, such as between at or about 0.01% and 5%, or between 0.1% and 1%, by weight, of the composition.

8. Volume of the Compositions

The provided compositions include low volume compositions. The low-volume compositions contain effective amounts of the active ingredients, e.g. the aminoalkane active ingredients, in relatively low volumes of liquid, such as, for example, at or about, or less than at or about, 5000, 4000, 3000, 2000, 1000, 900, 800, 700, 600 mL, and typically less than at or about 500, 450, 400, 350, 300, 250, 200, 150, 100, 75, 50, 40, 30, 20, 19, 18, 17, 16, 15, 14, 13, 12, 11, 10, 9, 8, 7, 6, 5, 4, 3, 2, 1, or fewer, mL, that are palatable compositions. For example, the compositions include low-volume compositions containing vasoconstrictor activity equivalent to the vasoconstrictor activity of between at or about 100 and 1000 micromoles of 2-amino-4-methylhexane HCl, e.g. at or about 100, 110, 120, 130, 140, 150, 160, 170, 180, 190, 200, 225, 250, 275, 280, 290, 300, 310, 320, 330, 340, 350, 360, 370, 380, 390, 400, 425, 426, 427, 428, 429, 430, 450, 500, 600, 700, 800, 900 or more micromoles of 2-amino-4-methylhexane HCl, or between at or about 1 g and 200 g, e.g. between at or about 10 g and 200 g, e.g. at or about 10, 11, 12, 13, 14, 15, 16, 17, 18, 19, 20, 21, 22, 23, 24, 25, 26, 27, 28, 29, 30, 40, 50, 55, 60, 65, 70, 75, 80, 85, 90, 95, 100, 101, 102, 103, 104, 105, 106, 107, 108, 109, 110, 111, 112, 113, 114, 115, 116, 117, 118, 119, 120, 125, 150 or 200 g, for example, between at or about 25 g and at or about 120 g, e.g. at or about 65 g 2-amino-4-methylhexane HCl. For example, the low-volume compositions include compositions having such vasoconstrictor activity in a volume of between at or about 1 and 10 mL, typically between 2 and 5 mL, such as 4 mL.

9. Palatability of the Compositions

The provided compositions typically are palatable compositions (typically palatable low-volume compositions), containing effective amounts of the active ingredients. The palatable compositions are tolerable, and typically desirable, for consumption by a subject, e.g. by oral administration, and have a pleasant (or not unpleasant) taste and/or smell. For example, provided are low-volume palatable compositions containing effective amounts of active ingredients, such as palatable compositions containing an appropriate amount of vasoconstrictor activity as described herein above. Palatability can be effected by adding one or more taste-modifying agents to the compositions, such as flavoring agents and sweetening agents as described herein.

10. Exemplary Compositions

Provided are compositions containing 2-amino-4-methylhexane HCL at a concentration of between at or about 3 mM and at or about 200 mM, such as between at or about 5 mM and at or about 150 mM, and typically at or about 107 mM (at or about 16.25 mg/mL), and taste-modifying agents, including sucralose and flavoring agents, such as flavoring agents that confer peach, peppermint, spearmint, mint, cinnamon and sour apple flavors. Provided are compositions containing 2-amino-4-methylhexane HCL; sucralose and flavoring agents (e.g. flavoring agents that confer peach, peppermint, spearmint, mint, cinnamon and sour apple flavors), at amounts sufficient to increase or enhance the palatability of the composition compared to the composition without the sucralose and flavoring agents; preservatives, such as potassium sorbate and sodium benzoate, typically a combination thereof, an emulsion stabilizer, e.g. a blend of gums, typically guar gum, xanthan gum and sodium alginate; other active ingredients, including caffeine, typically caffeine anhydrous, and conjugated linoleic acid (CLA); a TPGS surfactant (e.g. TPGS 1000); a phosphatidylcholine co-surfactant; and a pH adjusting agent, e.g. citric acid.

Also provided are compositions containing 2-amino-4-methylhexane HCL; sucralose and flavoring agents (e.g. flavoring agents that confer peach, peppermint, spearmint, mint, cinnamon and sour apple flavors), at amounts sufficient to increase or enhance the palatability of the composition compared to the composition without the sucralose and flavoring agents; preservatives, such as potassium sorbate and sodium benzoate, typically a combination thereof, an emulsion stabilizer, e.g. a blend of gums, typically guar gum, xanthan gum and sodium alginate; other active ingredients, including caffeine, typically caffeine anhydrous, conjugated linoleic acid (CLA), Vitamin B12 and chromium picolinate, a TPGS surfactant (e.g. TPGS 1000), a phosphatidylcholine co-surfactant, water and a pH adjusting agent, e.g. citric acid.

Also provided are compositions containing 2-amino-4-methylhexane HCL; sucralose and flavoring agents (e.g. flavoring agents that confer peach, peppermint, spearmint, mint, cinnamon and sour apple flavors), at amounts sufficient to increase or enhance the palatability of the composition compared to the composition without the sucralose and flavoring agents; preservatives, such as potassium sorbate and sodium benzoate, typically a combination thereof, an emulsion stabilizer, e.g. a blend of gums, typically guar gum, xanthan gum and sodium alginate; other active ingredients, including caffeine, typically caffeine anhydrous, conjugated linoleic acid (CLA), L-taurine and alpha lipoic acid, a TPGS surfactant (e.g. TPGS 1000), a phosphatidylcholine co-surfactant, water and a pH adjusting agent, e.g. citric acid.

In one example, the provided composition contains 2-amino-4-methylhexane HCL, sucralose, a peach flavoring agent, potassium sorbate, sodium benzoate, an emulsion stabilizer containing guar gum, xanthan gum, and sodium alginate, conjugated linoleic acid (CLA), Vitamin B12, chromium picolinate, caffeine anhydrous, a TPGS-1000 surfactant, a phosphatidylcholine co-surfactant, water and citric acid.

In one example, the provided composition contains 2-amino-4-methylhexane HCL, sucralose, a mint flavoring agent, potassium sorbate, sodium benzoate, an emulsion stabilizer containing guar gum, xanthan gum, and sodium alginate, conjugated linoleic acid (CLA), L-taurine, alpha lipoic acid, caffeine anhydrous, a TPGS-1000 surfactant, a phosphatidylcholine co-surfactant, water and citric acid.

Typically, the amount of 2-amino-4-methylhexane HCL is between at or about 3 mM and at or about 200 mM, between at or about 5 mM and at or about 110 mM, between at or about 100 mM and at or about 110 mM, and typically at or about 107 mM, or between at or about 1 mg/mL and 25 mg/mL, and typically at or about 16.25 mg/mL.

Typically, the amount of the flavoring agent is between at or about 0.4% and at or about 2%, typically between at or about 0.45% and at or about 1.5%, by weight, of the composition, e.g. at or about 1.5%, 0.45% or 1%, by weight, of the composition.

Typically, the amount of the sucralose is less than 2%, by weight of the composition, such as at or about 1.4%, by weight, of the composition, e.g. at or about 1.438%, by weight, of the composition.

Typically, the amount of the preservative (e.g. sodium benzoate and/or potassium sorbate) is between at or about 0.4% and at or about 0.8%, by weight, of the composition, and typically includes at or about 0.4%, by weight, sodium benzoate and at or about 0.4%, by weight, potassium sorbate.

Typically, the amount of the caffeine, e.g. caffeine anhydrous, is at or about 2%, by weight, of the composition, and the amount of the CLA is at or about 0.248%, by weight, of the composition.

Typically, the amount of the TPGS surfactant is between at or about 0.6% and at or about 1.25%, by weight, of the composition, such as between at or about 0.6% and at or about 0.8%, by weight of the composition, e.g. at or about 0.625%, 0.75%, or 1.25%, by weight, of the composition; and typically the amount of the phosphatidylcholine co-surfactant is at or about 0.075%, by weight, of the composition.

Typically, the amount of water is between at or about 89% and at or about 92%, by weight, of the composition, e.g. at or about 89%, at or about 90% or at or about 91%, e.g. at or about 89.5%, at or about 90.4% and at or about 91.3% by weight, of the composition.

Typically, the amount of the emulsion stabilizer is at or about 0.165%, by weight, of the composition and the amount of the pH adjusting agent is at or about 2.66%, by weight, of the composition.

Typically, the amount of chromium picolinate is between at or about 0 and at or about 0.015%, by weight, of the composition, e.g. at or about 0.015%, by weight, of the composition; typically, the amount of Vitamin B12 is between at or about 0% and at or about 0.025%, by weight, of the composition, such as at or about 0.025%, by weight, of the composition.

Typically, the amount of L-taurine is between at or about 0% and at or about 0.25%, by weight, of the composition, such as at or about 0.25%, by weight, of the composition; typically, the amount of alpha lipoic acid is between at or about 0% and at or about 0.025%, by weight, of the composition, such as at or about 0.025%, by weight, of the composition.

Provided are compositions containing 2-amino-4-methylhexane HCL at a concentration of at or about 16.25 mg/mL (at or about 107 mM); a flavoring agent (e.g. peach, spearmint, peppermint, mint, cinnamon or sour apple) at a concentration of between at or about 0.45% and at or about 1.5%, by weight, of the composition; sucralose at an amount of at or about 1.44%, by weight, of the composition; water, at an amount between at or about 89.5% and 90.4%, by weight, of the composition; potassium sorbate, at an amount of at or about 0.04%, by weight, of the composition; sodium benzoate, at an amount of at or about 0.04%, by weight, of the composition; an emulsion stabilizer (e.g. blend of guar gum, xanthan gum and sodium alginate), at an amount of at or about 0.165%, by weight, of the composition; caffeine anhydrous, at an amount of at or about 2%, by weight, of the composition; CLA, at an amount of at or about 0.248%, by weight of the composition; phosphatidylcholine at an amount of at or about 0.075%, by weight, of the composition; TPGS surfactant, at an amount between at or about 0.625% and at or about 1.25%, e.g. at or about 0.625%, 0.75%, or 1.25%, by weight, of the composition; and citric acid, at an amount of at or about 2.66%, by weight, of the composition.

Also provided are compositions containing 2-amino-4-methylhexane HCL at a concentration of at or about 16.25 mg/mL (at or about 107 mM); a flavoring agent (e.g. peach, spearmint, peppermint, mint, cinnamon or sour apple) at a concentration of between at or about 0.45% and at or about 1.5%, by weight, of the composition; sucralose at an amount of at or about 1.44%, by weight, of the composition; water, at an amount between at or about 89.5% and 90.4%, by weight, of the composition; potassium sorbate, at an amount of at or about 0.04%, by weight, of the composition; sodium benzoate, at an amount of at or about 0.04%, by weight, of the composition; an emulsion stabilizer (e.g. blend of guar gum, xanthan gum and sodium alginate), at an amount of at or about 0.165%, by weight, of the composition; caffeine anhydrous, at an amount of at or about 2%, by weight, of the composition; CLA, at an amount of at or about 0.248%, by weight of the composition; chromium picolinate, at an amount of at or about 0.015%, by weight, of the composition; Vitamin B12, at an amount of at or about 0.025%, by weight, of the composition; phosphatidylcholine at an amount of at or about 0.075%, by weight, of the composition; TPGS surfactant, at an amount between at or about 0.625% and at or about 1.25%, or between at or about 0.625% and at or about 0.75%, e.g. at or about 0.625% or 0.75% or 1.25%, by weight, of the composition; and citric acid, at an amount of at or about 2.66%, by weight, of the composition.

For example, provided are compositions containing 2-amino-4-methylhexane HCL at a concentration of at or about 16.25 mg/mL (at or about 107 mM); a peach flavoring agent at an amount (concentration) in the composition of at or about 0.663% by weight; sucralose, at an amount in the composition of at or about 1.438%, by weight; water, at an amount in the composition of at or about 90.388%, by weight; potassium sorbate, at an amount in the composition of at or about 0.04%, by weight; sodium benzoate, at an amount in the composition of at or about 0.04%, by weight; an emulsion stabilizer (blend of guar gum, xanthan gum and sodium alginate), at an amount in the composition of at or about 0.165%, by weight; caffeine anhydrous, at an amount in the composition of at or about 2%, by weight; CLA, at an amount in the composition of at or about 0.248%, by weight; phosphatidylcholine at an amount in the composition of at or about 0.075%, by weight; TPGS surfactant, at an amount in the composition of at or about 0.625%, by weight; Vitamin B12, at an amount in the composition of at or about 0.025%, by weight; chromium picolinate, at an amount in the composition of at or about 0.015%, by weight; and citric acid, at an amount of at or about 2.66%, by weight, of the composition.

Provided are compositions containing 2-amino-4-methylhexane HCL at a concentration of at or about 16.25 mg/mL (at or about 107 mM); a spearmint flavoring agent at an amount (concentration) in the composition of at or about 1.5% by weight; sucralose, at an amount in the composition of at or about 1.438%, by weight; water, at an amount in the composition of at or about 89.465%, by weight; potassium sorbate, at an amount in the composition of at or about 0.04%, by weight; sodium benzoate, at an amount in the composition of at or about 0.04%, by weight; an emulsion stabilizer (blend of guar gum, xanthan gum and sodium alginate), at an amount in the composition of at or about 0.165%, by weight; caffeine anhydrous, at an amount in the composition of at or about 2%, by weight; CLA, at an amount in the composition of at or about 0.248%, by weight; phosphatidylcholine at an amount in the composition of at or about 0.075%, by weight; TPGS surfactant, at an amount in the composition of at or about 0.75%, by weight; and citric acid, at an amount of at or about 2.66%, by weight, of the composition.

Provided are compositions containing 2-amino-4-methylhexane HCL at a concentration of at or about 16.25 mg/mL (at or about 107 mM); a sour apple flavoring agent at an amount (concentration) in the composition of at or about 0.45% by weight; sucralose, at an amount in the composition of at or about 1.438%, by weight; water, at an amount in the composition of at or about 90.515%, by weight; potassium sorbate, at an amount in the composition of at or about 0.04%, by weight; sodium benzoate, at an amount in the composition of at or about 0.04%, by weight; an emulsion stabilizer (blend of guar gum, xanthan gum and sodium alginate), at an amount in the composition of at or about 0.165%, by weight; caffeine anhydrous, at an amount in the composition of at or about 2%, by weight; CLA, at an amount in the composition of at or about 0.248%; by weight; phosphatidylcholine at an amount in the composition of at or about 0.075%, by weight; TPGS surfactant, at an amount in the composition of at or about 0.75%, by weight; and citric acid, at an amount of at or about 2.66%, by weight, of the composition.

Provided are compositions containing 2-amino-4-methylhexane HCL at a concentration of at or about 16.25 mg/mL (at or about 107 mM); a peppermint flavoring agent at an amount (concentration) in the composition of at or about 1% by weight; sucralose, at an amount in the composition of at or about 1.438%, by weight; water, at an amount in the composition of at or about 89.965%, by weight; potassium sorbate, at an amount in the composition of at or about 0.04%, by weight; sodium benzoate, at an amount in the composition of at or about 0.04%, by weight; an emulsion stabilizer (blend of guar gum, xanthan gum and sodium alginate), at an amount in the composition of at or about 0.165%, by weight; caffeine anhydrous, at an amount in the composition of at or about 2%, by weight; CLA, at an amount in the composition of at or about 0.248%, by weight; phosphatidylcholine at an amount in the composition of at or about 0.075%, by weight; TPGS surfactant, at an amount in the composition of at or about 0.75%, by weight; and citric acid, at an amount of at or about 2.66%, by weight, of the composition.

Provided are compositions containing 2-amino-4-methylhexane HCL at a concentration of at or about 16.25 mg/mL (at or about 107 mM); a cinnamon flavoring agent at an amount (concentration) in the composition of at or about 1% by weight; sucralose, at an amount in the composition of at or about 1.438%, by weight; water, at an amount in the composition of at or about 89.965%, by weight; potassium sorbate, at an amount in the composition of at or about 0.04%, by weight; sodium benzoate, at an amount in the composition of at or about 0.04%, by weight; an emulsion stabilizer (blend of guar gum, xanthan gum and sodium alginate), at an amount in the composition of at or about 0.165%, by weight; caffeine anhydrous, at an amount in the composition of at or about 2%, by weight; CLA, at an amount in the composition of at or about 0.248%, by weight; phosphatidylcholine at an amount in the composition of at or about 0.075%, by weight, TPGS surfactant, at an amount in the composition of at or about 0.75%, by weight; and citric acid, at an amount of at or about 2.66%, by weight, of the composition.

Provided are compositions containing 2-amino-4-methylhexane HCL at a concentration of at or about 16.25 mg/mL (at or about 107 mM); a mint flavoring agent at an amount (concentration) in the composition of at or about 1.09%, by weight; sucralose, at an amount in the composition of at or about 1.438%, by weight; water, at an amount in the composition of at or about 91.86%, by weight; potassium sorbate, at an amount in the composition of at or about 0.04%, by weight; sodium benzoate, at an amount in the composition of at or about 0.04%, by weight; an emulsion stabilizer (blend of guar gum, xanthan gum and sodium alginate), at an amount in the composition of at or about 0.165%, by weight; caffeine anhydrous, at an amount in the composition of at or about 2%, by weight; CLA, at an amount in the composition of at or about 0.248%, by weight; phosphatidylcholine at an amount in the composition of at or about 0.075%, by weight; TPGS surfactant, at an amount in the composition of at or about 1.25%, by weight; and citric acid, at an amount of at or about 0.17%, by weight, of the composition.

Also provided are compositions containing 2-amino-4-methylhexane HCL at a concentration of at or about 16.25 mg/mL (at or about 107 mM); a flavoring agent (e.g. peach, spearmint, peppermint, mint, cinnamon or sour apple) at a concentration of between at or about 0.45% and at or about 1.5%, by weight, of the composition; sucralose at an amount of at or about 1.44%, by weight, of the composition; water, at an amount between at or about 89.5% and 91.3%, by weight, of the composition; potassium sorbate, at an amount of at or about 0.04%, by weight, of the composition; sodium benzoate, at an amount of at or about 0.04%, by weight, of the composition; an emulsion stabilizer (e.g. blend of guar gum, xanthan gum and sodium alginate), at an amount of at or about 0.165%, by weight, of the composition; caffeine anhydrous, at an amount of at or about 2%, by weight, of the composition; CLA, at an amount of at or about 0.248%, by weight of the composition; L-taurine, at an amount of at or about 0.25%, by weight, of the composition; alpha lipoic acid, at an amount of at or about 0.025%, by weight, of the composition; phosphatidylcholine at an amount of at or about 0.075%, by weight, of the composition; TPGS surfactant, at an amount between at or about 0.625% and at or about 1.5%, or between at or about 0.625% and at or about 0.75%, e.g. at or about 0.625% or 0.75% or 1.5%, by weight, of the composition; and citric acid, at an amount of at or about 2.66%, by weight, of the composition.

For example, provided are compositions containing 2-amino-4-methylhexane HCL at a concentration of at or about 16.25 mg/mL (at or about 107 mM); a mint flavoring agent at an amount (concentration) in the composition of at or about 1.09%, by weight; sucralose, at an amount in the composition of at or about 1.438%, by weight; water, at an amount in the composition of at or about 91.332%, by weight; potassium sorbate, at an amount in the composition of at or about 0.04%, by weight; sodium benzoate, at an amount in the composition of at or about 0.04%, by weight; an emulsion stabilizer (blend of guar gum, xanthan gum and sodium alginate), at an amount in the composition of at or about 0.165%, by weight; caffeine anhydrous, at an amount in the composition of at or about 2%, by weight; CLA, at an amount in the composition of at or about 0.248%, by weight; L-taurine, at an amount in the composition of at or about 0.25%, by weight; alpha lipoic acid, at an amount in the composition of at or about 0.025%, by weight; phosphatidylcholine at an amount in the composition of at or about 0.075%, by weight; TPGS surfactant, at an amount in the composition of at or about 1.5%, by weight; and citric acid, at an amount of at or about 0.17%, by weight, of the composition.

### C. METHODS FOR PRODUCING THE COMPOSITIONS

Also provided are methods for making the provided compositions. Equipment for use in the methods and general steps of the methods are described below. The methods include bench-top manufacturing processes, which are used to make small quantities of the compositions. The methods also include scaled-up manufacturing processes, which are used to make larger batches of the compositions. Any of the bench-top processes can be scaled up to perform the methods using the scaled-up processes. Any of the provided compositions can be made using either scaled-up or bench-top processes.

1. Equipment for Use in the Methods

Equipment, for example, vessels for mixing the water and oil phases, and the composition, for example, tanks; scales; mixers, including standard mixers and homogenizers; heating and cooling apparatuses, including water-jacketed tanks, hot plates, water baths and chillers (coolers), including recirculating coolers; transfer apparatuses, for example, transfer means, for example, pumps, hoses, sanitary fittings; ball valves; purifiers, for example, filters, for example, carbon filters, ion exchange equipment, reverse osmosis equipment, end-point filters and end product filters; evaluation means, for example, pH and temperature meters; and other equipment, is used in various steps of the provided methods for making the compositions. The choice of equipment depends on a plurality of factors, including batch size and manufacturing process.

a. Scales

One or more scales can be used to measure the amount of the ingredients before adding them to the appropriate vessel. Alternatively, the ingredients can be weighed in the vessel, for example, in a tank on top of a scale.

Any of a plurality of well-known, commercially sold scales can be used to weigh the ingredients. Choice of scale(s) can depend on a number of factors, including the mass of the composition (e.g. the batch size) being made and the ingredient being weighed. In one example, multiple scales are used to weigh the various ingredients of the compositions. In general, relatively larger capacity (weight) scale(s) are used in making larger batches of the compositions while relatively smaller capacity scale(s) are used in making smaller batches.

Exemplary of the scales used with the provided methods to weigh the ingredients are a Toledo Scale (Model GD13×/USA); a Sartorius Basic Analytical Scale (Model BA110S) which is a basic series analytical scale with a 110 g capacity and a resolution of 0.1 mg; and an OHAUS Scale (Model CS2000), which is a compact portable digital scale having a 2000 g capacity and a resolution of 1 g.

b. Purifiers

Purifiers, such as filters, are used in the provided methods to remove impurities from the ingredients prior to their addition to the composition and/or from the composition or a phase of the composition. For example, the water added to the water phase typically is purified water. In one example, one or more purifiers, for example, carbon filters, ion exchange purifiers, reverse osmosis purifiers, and/or end point filters are used to filter water, for example, city water, prior to its addition to the water phase, for example, to remove impurities, for example, sediment, from the water.

Purifiers that can be used with the provided methods include filters, for example, 100 micron filters and carbon filters, which are filters that use activated carbon to remove impurities by chemical adsorption. Carbon filtering typically is used for water purification and are particularly effective at filtering out chlorine, sediment, volatile organic compounds and other impurities. Typically, the particles removed by carbon filters are between about 0.5 microns and about 50 microns. Other filters are well known and can be used with the provided methods.

The purifiers also include reverse osmosis purifiers, which use mechanical pressure to purify liquids, for example, water. In one example, the pressure forces the water through a semi-permeable membrane to remove impurities.

The purifiers also include exchange purifiers, for example, an ion exchange purifier using a resin bed, for example, a zeolite resin bed, to replace salts, e.g. cations, for example, magnesium and calcium, with other cations, for example, sodium and potassium cations. Such purifiers can be purchased, for example, from Aquapure Filters, Clarkston, Mich.

In one example, the purifier is an end product filter (e.g. a 100 micron FSI filter, Product Number BPEM 100-5GP). This filter is used to filter any impurities out of the final product (e.g. the final pre-emulsion composition). Other filters are known and can be used with the provided methods.

c. Vessels

One or more, typically two or more, vessels, for example, tanks, for example, water-jacketed tanks; pots; and/or beakers, for example, Pyrex® beakers, are used in the provided methods to contain the ingredient(s) of the provided compositions, for example, during mixing and/or heating or cooling. Typically, separate vessels (an oil phase tank and a water phase tank) are used for mixing and heating the ingredients of the oil phase and the water phase, prior to combining the two phases. In another example, an additional vessel, for example, a holding and/or packaging tank, is used for holding and/or packaging the compositions and/or for addition/mixing of additional ingredients to the compositions.

A number of vessels are available for mixing ingredients. Typically, the vessels are cleaned, for example, rinsed, soaped and/or sanitized according to known procedures, prior to use and between uses, such as with the cleaning procedures described below.

In the bench-top process, the vessel is a container, for example, a bench-top container, for example, flasks, beakers, for example, Pyrex® beakers, vials, measuring containers, bottles and/or other bench-top containers.

In the scaled-up manufacturing process, the vessels are tanks, for example, water phase tanks, oil phase tanks and holding/packaging tanks. Typically, the tanks are equipped with one or more mixers, for example, a standard mixer and/or homogenizer, which are used to mix the ingredients added to the tank. In one example, the tank further is equipped with a heating and/or cooling device. For example, the tank can be a water-jacketed tank. The temperature of the water-jacketed tank is controlled through the water-jacket, for example, to heat the contents, for example, while mixing.

Exemplary of the tanks that can be used with the provided methods are water-jacketed tanks, for example, the Overly 550 Gallon water jacketed tank (Model 10576501G), which has a 550 gallon capacity and typically is used as a water-phase tank, the Schweitzers 450 gallon tank (Model #5214-C), which has a 450 gallon capacity and typically is used as an oil phase tank and the Royal 190 gallon water jacketed tank (Model 9977-5), which has a 190 gallon capacity and can be used as a water or oil phase tank when mixing smaller volumes. Other tanks are well known and can be used with the provided methods for mixing the compositions, for example, the phases of the compositions.

d. Mixers

Mixers are used in the provided methods to blend, mix and/or emulsify the compositions and ingredients, mixtures and phases of the compositions. In one example, the mixers are used to keep the ingredients and/or mixture circulating to maintain temperature, viscosity and/or other parameters of the mixture. The mixers include, but are not limited to, standard mixers, for example, standard mixers, which can be used, for example, to mix ingredients and maintain a homogeneous mixture, such as while heating a mixture of ingredients. Exemplary of the standard mixers is a LIGHTNING® mixer (LIGHTNIN, Rochester, N.Y.), for example, Model Numbers XJC117 and ND-2. In one example, the LIGHTNIN® mixers are fixed-mount, gear drive high-flow mixers, for use with closed tanks. Another example of a standard mixer is a mixer sold by IKA®, for example, overhead IKA® mixers, for example, model Nos. RW-14 Basic and RE-16S, which are laboratory stirrers and can be used to mix ingredients. In one example, the mixer(s) are attached to the vessels, for example, the tanks, for example, mounted or clamped onto the tanks, for example, the top of the tanks. In another example, the mixers are placed in the vessels for mixing.

The mixers also include homogenizers, which are used, for example, to emulsify mixtures, such as phases, e.g. oil and water phases, in order to form an emulsion, e.g. for mixing the oil and water phases after combining the phases. The homogenizers provide high shear dispersion of solids and emulsification of immiscible liquids at high shear rates. The homogenizers include, but are not limited to, high-shear homogenizers, for example, reverse homogenizers sold by Arde Barinco, Inc., Norwood, N.J., for example, Model CJ-50, which is a 3600 rpm mixer having a 6 inch rotor diameter, a tip speed of 5575 ft/minute and an emersion depth of 33 inches and has six separate openings at the bottom and top, which concentrates the liquid into six chambers, reducing the surface volume and creating a shear effect; and Model CJ-4E, which is a 10,000 rpm mixer with fan-cooled motor, optimized for 1 to 5 gallon batch sizes, having a 1.875 inch rotor diameter, a tip speed of 4920 rpm and an immersion depth of 16 inches. The homogenizers further include other homogenizers, for example, other reversible homogenizers sold by Arde Barinco Inc.

In one example, the homogenizer is attached to the top of the vessel, for example, the tank, for example, by clamps or by channel locks and an electrical hoist. In another example, the homogenizer is placed in the vessel. The Arde Barinco reversible homogenizers contain axial flow impellers, which create two distinct mixing actions, depending on direction. Downward “vortex flow” pulls solids from top and bottom of the mixture, while upward “umbrella flow” controls mixing at the highest shear and recirculation rates without splashing or incorporation of air. The reversible homogenizers typically are equipped with an adjustable baffle plate, which can be adjusted to control the type of mixing, for example at different times during mixture, e.g. emulsification.

A number of other mixers are well known and can be used with the provided methods. Exemplary of the mixers that can be used with the provided methods are homogenizers, inline mixers/mixing, Ribbon, Plow/Paddle Blenders Forberg Mixers, Conveyors, Bag Dumps & Compactors, V-Blenders, Blade Mixers, Double Cone Mixers, Continuous Mixers, Speedflow Mixers, Batch Mixers, Double Ribbon Blenders, Paddle and Ribbon Mixers with Choppers, Plow Blenders/Turbulent Mixers, Fluidizing Forberg-Type Mixers, Air Mixers, Active Mixers, Passive Mixers, Top Entry Mixers, Side Entry Mixers, Static Mixers, Fixed Entry Mixers, Portable Mixers—direct and gear drive, Sanitary Mixers, Drum Mixers, Bulk Container (IBC) Mixers, Lab Stirrers, Variable Speed Mixers, dough mixer, vertical mixer, spiral mixer, twin arm mixer, fork mixer, double spiral mixer, all agitators, agitator mixers, Banbury Mixers, Rubber Mixers, Blondheim Mixers, Chum Mixers, Conical Mixers, Continuous Mixers, Disperser Mixers, Pan Mixers, Emulsifier Mixers, Hobart Mixers, Liquifier Mixers, Littleford Mixers, Meat Mixers, Plow Mixers, Mixmuller Mixers, Nauta Mixers, Oakes Mixers, Planetary Mixers, Pony Mixers, PUG Mixers, Ribbon Mixers, Ross Mixers, Rotary Mixers, Sigma Mixers, Single Arm Mixers, Tote Bin Mixers, Tumble Mixers, Vacuum Mixers, Turbolizer Mixers, Twin Shell Mixers, V-Type Mixers, Zig-Zag Mixers side arm mixers, hand-held mixers, stir rods, stir bars, magnetic mixers and overhead mixers, for example, mechanical and/or electric overhead mixers.

e. Heating/Cooling Apparatuses

Equipment for practicing the provided methods includes heating and cooling apparatuses, for controlling the temperature of ingredients and combinations thereof, while generating the compositions.

Typically, the heating apparatuses are capable of heating the mixture to between 45° C. or about 45° C. and 85° C. or about 85° C., for example, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84 or 85° C., and typically is used to heat the mixtures to low heat, such as at or about 60° C.

The heating apparatuses include water-jackets, for example, water jackets on water-jacketed tanks, which can be controlled, for example, using a control panel, to adjust the temperature of the contents of the tank. Other heating apparatuses include immersible and/or submersible heaters, for example, 12 KW or 13 KW sanitary heaters, including food-grade heaters, that are immersed into the tanks, typically while mixing and typically are used when high heat is required, for example, temperature greater than 60° C. or about 60° C., or greater than 80° C. or about 80° C. The heating apparatuses also include stoves, for example, propane stoves, hot plates, for example, the Thermolyne hot plate, model number 846925 and model number SP46615.

The cooling apparatuses include apparatuses that can cool the ingredients and combinations thereof, for example, rapidly cooling and/or cooling while mixing the ingredients. Typically, the cooling apparatus cools the liquid to between 25° C. or about 25° C. and 45° C. or about 45° C., for example, 25, 26, 27, 28, 29, 30, 31, 32, 33, 34, 35, 36, 37, 38, 39, 40, 41, 42, 43, 44 or 45° C., and typically between at or about 30° C. and 35° C. Typically, the cooling is rapid cooling, for example, cooling to between at or about 30° C. and at or about 35° C. in between at or about 15 minutes and at or about 2 hours, typically, between at or about 30 minutes and at or about 60 minutes, for example, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59 or 60 minutes.

The cooling apparatuses include chillers, for example, recirculating coolers, which can be attached to the vessel, for example, remotely or by a tank mounted in the cooler, to repeatedly circulate fluid from the tank, through the chiller and back to the vessel, to rapidly cool and maintain the temperature of the mixture during mixing. Exemplary of an open-loop chiller that can be attached to the tank and used with the provided methods are chillers sold by Turmoil, West Swanzey, N.H., for example, open or closed-loop coolers, for example, model No. OC-1000 RO. Other cooling apparatuses are well known and can be used with the provided methods.

The cooling apparatuses also include water baths and ice baths, for example, water baths and/or ice baths in which the vessel(s) are placed, for example, during homogenizing.

f. Transfer Means

Transfer means are used with the provided methods to transfer liquid from one vessel to another vessel, typically to transfer the water phase to the oil phase vessel or to transfer the oil phase to the water phase vessel, to combine the phases and form the emulsion. Transfer means include transfer pumps and associated accessories (e.g. fittings), for example, ball valves, sanitary fittings (for example, sanitary fittings sold by Granger, Inc., Lake Forrest II) and transfer hoses (for example, hoses sold by Sani-Tech West, Oxnard, Calif., such as the food grade Sani-Tech® STHT-R-HD Braid-Reinforced Heavy Duty Silicone Hose), for example, food grade hoses attached to the transfer pumps. Suitable transfer pumps include the Teel Pump (Model 2P377B), Granger, Inc. Lake Forrest II, a self-priming pump having a power rating of 2 HP, 60 Hz voltage 208-230/460 AC, speed of 3450 rpm, and other pumps, such as self-priming pumps from Grainger, Inc. The transfer means also include means for manually transferring the liquid to another vessel, for example, by pouring, pipetting and/or other well-known methods of manually transferring liquids.

g. Evaluation Equipment

Evaluation equipment includes equipment used to evaluate properties of the compositions and/or phases of the compositions, such as the temperature, pH, clarity, color, activity, smell and/or taste of the compositions. The evaluation equipment include pH and temperature meters, such as the pH and temperature sold by Hanna Instruments, (model number HI 8314), which can be used to measure the temperature and the pH of the compositions. Temperature meters also include temperature probes, for example, digital and/or water-proof temperature probes, for example, temperature probes sold by Cooper-Atkins, Middlefield, Conn., such as the Cooper-Atkins digital waterproof temperature probe (Model # DPP400W). The compositions typically also are evaluated/analyzed to verify they meet industry standards, such as to verify the amounts of the active ingredients, and contain levels of microbials and heavy metals that are not above acceptable levels. Typically, these tests are performed by sending a sample of the composition to a commercial testing facility, as described in section 2(g), below.

2. General Methods for Producing the Compositions

In general, the provided methods for making the provided compositions are performed by generating an oil phase, generating a water phase and combining (e.g. using transfer means) and mixing the phases, to form emulsions. The oil and water phases typically are generated in separate vessels (e.g. tanks). Generation of the water phase and generation of the oil phase can be performed simultaneously or sequentially, in any order. Typically, both phases are heated to a desired temperature (e.g. 60° C.) prior to combining the phases. The methods typically include additional steps, such as evaluating properties of the compositions, adding additional ingredients (e.g. taste-modifying agents), packaging and/or filtering. The provided methods can be performed with a bench-top manufacturing process (for small batch sizes) or performed with a scaled-up manufacturing process (for larger batch sizes). Each of the provided compositions can be made with either the bench-top or scaled up process. In one example, the composition is first made with the bench-top process and then the method scaled up to make larger quantities of the composition.

The bench-top process is performed on a bench, counter, table or other surface. Typically, the bench-top process is used to make emulsions having relatively smaller volumes than those made with the scaled-up process, for example, volumes less than 1 L or about 1 L or less than 1 gallon or about 1 gallon, for example, less than about 500 mL, for example, 1000, 900, 800, 700, 600, 500, 450, 400, 350, 300, 250, 200, 150, 100, 50 or less.

For the bench-top process, the equipment typically is sufficiently compact to be used on a bench top or other similar surface, typically sufficiently compact to be moved, for example, lifted, by the artisan using the methods. For example, the vessels, for example, water phase vessels, oil phase vessels, holding vessels, and packaging vessels typically are bench-top vessels, for example, flasks, beakers, vials, measuring containers, bottles and/or other bench-top containers. In one example, the vessel in the bench-top process is a Pyrex® beaker.

Typically, the mixers for use in the bench-top processes of the methods are mixers that can be used in the bench-top vessels, for example, standard mixers, including hand-held mixers, stir rods, stir bars, magnetic mixers and overhead mixers, for example, mechanical and/or electric overhead mixers and/or other mixers that can be used in the vessels. Suitable bench-top mixers include standard mixers, for example, standard mixers sold by IKA®, for example, overhead IKA® mixers, for example, model Nos. RW-14 Basic and RE-16S, which are laboratory stirrers and can be used to mix ingredients, for example, to generate the oil and water phases. Suitable bench-top mixers also include homogenizers, for example, reversible homogenizers, including The Arde Barinco reversible homogenizer, Model no. CJ-4E, which can be used to emulsify the phases.

Typically, the heating and cooling apparatuses are those that can be used with the bench-top vessels, for example, hot plates, ice baths and/or water baths into (or onto) which the vessels can be placed, for example, for rapid cooling. The evaluation means used in the bench-top process, for example, the temperature and/or pH meters, typically are capable of being placed in the bench-top vessels.

For the bench-top process, combining the oil and water phases typically is carried out manually, e.g. by pouring, pipetting and/or another manual transfer means.

The scaled-up manufacturing process of the methods typically is used to make compositions of relatively larger volumes, such as volumes greater than 1 L or about 1 L or greater than 1 gallon or about 1 gallon, for example, greater than about 500 mL, for example, at least 0.5 L, 1 L, 2 L, or 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, 50, 60, 70, 80, 90, 100, 150, 200, 250, 300, 350, 400, 450, 500, 550, 600, 650, 700, 800, 900, 1000 or more gallons.

In general, equipment used for the scaled-up process is compatible with these larger volume batches (batch sizes). For example, the vessels for use in the scaled-up processes typically are tanks, for example, water jacketed tanks, which are equipped with water jackets that can be used as heating apparatuses to heat the oil and water phase ingredients during generation of the oil and water phases. The water jackets typically are controlled via control panels. The transfer means typically include devices attached to and connecting the tanks, such as transfer pumps and associated fittings, for example, ball valves and hoses that are attached to the tanks. Mixers for use in the scaled-up process include standard mixers, for example, mounted mixers, for example LIGHTNIN® mixers, such as Model XJC117 (a fixed-mount, gear drive high-flow mixer), and Model ND2.

Prior to beginning the methods, water-jacket lines on any water jacketed oil phase and water phase tanks typically are bled. The water-jacket switches then are turned on to maintain a pressure in the water jackets of between at or about 20 and at or about 40 PSI (pounds per square inch). If the pressure in the water jacket falls below 20 PSI during the method, the line is bled and checked for bubbles while purging the line.

a. Water Phase Ingredients

The water phase includes water and typically, other water phase ingredients, e.g. hydrophilic and/or amphipathic ingredients of the compositions. Water phase ingredients include water, typically purified water (e.g. purified as provided herein); water-soluble vitamins (e.g. Vitamin B12); water-soluble supplements (e.g. chromium picolinate) and/or minerals; preservatives (e.g. potassium sorbate and/or sodium benzoate); water-soluble or amphipathic active ingredients, such as the aminoalkane active ingredients (e.g. 2-amino-4-methylhexane or acid addition salt thereof, e.g. HCl salt, or equivalent) and other water-soluble or amphipathic active ingredients, such as stimulants such as caffeine, e.g. caffeine anhydrous; pH adjusters; emulsion stabilizers (e.g. SALADIZER® brand emulsion stabilizer) and other amphipathic ingredients such as surfactants (e.g. TPGS) and co-surfactants (e.g. phosphatidylcholine). The amphipathic ingredients can be included in the water phase, the oil phase, or both.

b. Water Phase Production

To produce the water phase, appropriate amounts of the water phase ingredients are added to the water phase vessel. Water phase vessels include tanks, for example, water-jacketed tanks such as, but not limited to, the Overly 550 gallon water jacketed tank or other tank described herein. The amounts of the water phase ingredients are measured, e.g. weighed, either prior to adding to the water phase vessel or are measured in the water phase vessel. In one example, the water phase ingredients are measured by weighing the ingredients on a scale (e.g. one or more of the scales described herein; choice of scale depends on the desired amount of the ingredient), before addition to the water phase vessel (e.g. any vessel described herein). Typically, the appropriate amount of the water phase ingredient is calculated based on the desired concentration (e.g. weight by weight (w/w) or molarity (M), volume by weight (v/w) or volume by volume (v/v)), of the ingredient in the final composition.

Water phase ingredients include water, typically purified water. In one example, unpurified water, for example, city water, is purified to remove impurities with one or more purifiers (e.g. with purifiers described herein) prior to adding it to the water phase vessel. In one example, the water (e.g. unpurified water such as city water) is purified by passing the water through the following purifiers, typically sequentially, in the following order: a carbon filter, an ion exchange purifier, a reverse osmosis purifier and an end-point filter, for example, a 100 micron end-point filter.

In general, the water phase ingredients are added, mixed and/or heated in the water phase vessel, such as a water phase tank, e.g. a water-jacketed tank, such as one of the tanks described herein (e.g. an Overly 550 gallon water jacketed tank or other tank). In one example, ingredients are heated to a low heat, e.g. between about 45° C. or about 45° C. and 85° C. or about 85° C., for example, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84 or 85° C., typically, 60° C. or 60° C., for example, by adjusting the temperature on a water-jacketed tank or using another heating apparatus.

The mixing is carried out with a standard mixer or a homogenizer or other mixer, such as, but not limited to, mixers described herein, for example, a LIGHTNIN® mixer (for example, model no. XJC117, a fixed-mount gear drive high-flow mixer) or a homogenizer, such as the Arde Barinco reversible homogenizer, Model No. CJ-4E), attached to the top of the water phase vessel, attached to the tank, for example, mounted on the top of the tank.

The water phase ingredients can be added to the water phase simultaneously or sequentially in any order. Typically, the purified water is added before adding the other water phase ingredients. In one example, one or more of the ingredients are mixed and/or heated in the water phase tank before adding the other water phase ingredients. In one example of the provided methods, the water phase is generated as follows:

Preservatives (e.g. potassium sorbate and/or sodium benzoate or other preservatives), water-soluble vitamins, if any (e.g. Vitamin B12), and other water-soluble supplements, if any (e.g. chromium picolinate), first are added to the water phase vessel and the ingredients are heated to the low temperature (typically at or about 60° C.). After the combination of water phase ingredients reaches the low heat, e.g. 60° C., an emulsion stabilizer, such as the SALADIZER® brand emulsion stabilizer (blend of xanthan gum, guar gum and sodium alginate) is added to the water phase and the ingredients mixed until dispersed, using a mixer, such as those described herein, for example, a homogenizer, such as the Arde Barinco reversible homogenizer, Model No. CJ-4E), attached to the top of the water phase vessel (e.g. tank), typically using the “forward” setting. Typically the heat is maintained at 60° C. After the ingredients are dispersed, the aminoalkane active ingredient (e.g. 2-amino-4-methylhexane or acid addition salt thereof, e.g. 2-amino-4-methylhexane HCl) then is added at the low heat, e.g. 60° C., followed by other active ingredients, such as other stimulants, if any, e.g. caffeine anhydrous. Typically, the ingredients are mixed until combined and maintained at the low heat, e.g. 60° C., until combining with the oil phase.

c. Oil Phase Ingredients

The oil phase ingredients include hydrophobic and/or amphipathic ingredients of the compositions. For example, oil phase ingredients include fatty acids (e.g. conjugated linoleic acid (CLA), fat-soluble vitamins, surfactants (e.g. TPGS) and co-surfactants (e.g. phosphatidylcholine) and other hydrophobic and/or amphipathic ingredients of the compositions.

d. Oil Phase Production

To produce the oil phase, appropriate amounts of the oil phase ingredients are added to the oil phase vessel (e.g. oil phase tank). Oil phase tanks include water-jacketed tanks, such as, but not limited to, the Royal 190 Gallon water jacketed tank and other tank described herein. The amounts of the oil phase ingredients are measured, e.g. weighed, either prior to adding to the vessel or are weighed/measured in the oil phase vessel. In one example, the oil phase ingredients are measured by weighing the ingredients on a scale (e.g. one or more of the scales described herein; choice of scale depends on the desired amount of the ingredient), before addition to the oil phase vessel (e.g. any vessel described herein). Typically, the appropriate amount of the oil phase ingredient is calculated based on the desired concentration (e.g. weight by weight (w/w) or molarity (M), volume by weight (v/w) or volume by volume (v/v)), of the ingredient in the final composition.

In general, the oil phase ingredients are added, mixed and/or heated in the water phase vessel. Mixing the oil phase ingredients is carried out with a standard mixer or other mixer, such as, but not limited to, mixers described herein, for example, a LIGHTNIN® mixer (for example, model no. XJC117, a fixed-mount gear drive high-flow mixer). Heating the oil phase ingredients is carried out using a heating apparatus, such as one described herein, typically a water-jacket on a water-jacketed tank. In one example, the ingredients are heated to a low heat, e.g. between about 45° C. or about 45° C. and 85° C. or about 85° C., for example, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84 or 85° C., typically, 60° C. or 60° C., for example, by adjusting the temperature on a water-jacketed tank.

The oil phase ingredients can be added to the oil phase vessel simultaneously or sequentially in any order. In one example, one or more of the ingredients are added, mixed and/or heated, prior to the addition of the other ingredients to the vessel. In one example, the oil phase is generated as follows:

Oils, if any (e.g. fatty acids, such as CLA) and co-surfactant, if any, (e.g. phosphatidylcholine) are added to the oil phase vessel and heated, for example, to a low heat (typically at or about 60° C.), for example, by adjusting the temperature on a water-jacketed tank, until dissolved. Surfactant(s), if any (e.g. TPGS) then are added at the low heat, e.g. 60° C. In one example, one or more oil phase ingredients is heated, e.g. to the low heat temperature, prior to addition to the oil phase. In one example, the oil phase ingredients are mixed (e.g. using a mixer as provided herein) during generation of the oil phase. Typically, the temperature of the oil phase is maintained at the low heat, e.g. 60° C., prior to combining with the water phase.

e. Combining Phases

After generation of the oil phase and the water phase, the phases are combined using transfer means, and mixed, e.g. homogenized, to form an emulsion. In one example, oil phases are transferred to water phase vessels. In another example, water phases are transferred to oil phase vessels. In another example, oil and water phases are transferred to another vessel, such as an emulsifying vessel.

Transfer means include any means for transferring the contents of one vessel to another vessel as described above, for example, transfer pumps and associated equipment, such as, but not limited to, combinations of sanitary fittings, hoses and/or ball valves; and manual transfer means, for example, pouring and/or pipetting means or other known transfer means. Typically, the phases are kept clean, e.g. sterile, during transfer; sterility can be maintained with transfer means having sanitary fittings and/or by combining the phases in a sterile environment. In one example, the transfer means include a transfer pump (e.g. a Teel pump, model 2P377B, sold by Granger, Inc.), sanitary fittings, transfer hoses (e.g. food grade hoses sold by Sani-Tech West) and ball valves, which are attached to the tanks and connect the tanks.

Simultaneous and/or subsequent to the combination of the phases, a mixer, e.g. homogenizer (e.g. a reversible homogenizer), is turned on in order to emulsify the water and oil phases. In one example, the homogenizer mounted on one of the tanks is turned on; the ball valves then are opened and the transfer pump turned on, to effect transfer of the contents of one tank to another (e.g. the oil phase to the water phase tank). As the phases are combined, they are mixed with the homogenizer to form an emulsion. The homogenizer can be adjusted, for example by adjusting the baffle plate to achieve and maintain the emulsion, e.g. by moving the baffle plate further into/out of the mixture. Typically, the phases are homogenized by operating the mixer (e.g. homogenizer) at a speed sufficient to form an emulsion. In one example, homogenizing is carried out at between at or about 1000 and 1500 rpm. Mixing typically is continued until the phases are combined, typically in an emulsion.

f. Cooling

The emulsion can be cooled during mixing to promote stability and emulsification, for example, by preventing or minimizing oxidization. The cooling can be rapid cooling and typically is performed using one or more cooling apparatuses, for example, any of the cooling apparatuses described herein or any known cooling apparatus. For example, the cooling apparatuses include recirculating coolers and water and ice baths. When the cooling apparatus is a recirculating cooler (e.g. Model No. OC-1000 RO, sold by Turmoil, West Swanzey, N.H.), fluid from the vessel containing the combined oil and water phases is circulated through the cooler, typically while mixing, and then back to the vessel, to rapidly cool and maintain the temperature of the mixture during mixing. Typically, the forming emulsion is mixed and cooled until the phases are emulsified and the temperature reaches between 25 or about 25° C. and 43 or about 43° C., typically between at or about 30° C. and 35° C., for example, 25, 26, 27, 28, 29, 30, 31, 32, 33, 34, 35, 36, 37, 38, 39, 40, 41, 42 or 43° C. Typically, when the cooling is rapid cooling, the temperature is reached in less than 2 hours or about 2 hours, typically less than 1 hour or about 1 hour, for example, in at least between 30 minutes or about 30 minutes and 60 minutes or about 60 minutes, for example, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59 or 60 minutes.

Cooling can be performed before or after additional steps, such as adding additional ingredients and/or evaluation of the composition. In one example, the cooling is carried out during or after addition of taste-modifying and/or pH adjusting agents.

g. Filtration, Additions, Evaluation and Packaging

After combining the oil and water phases to form a mixture (emulsion phase), one or more additional steps can be carried out to evaluate, analyze and/or package the composition. Typically, taste-modifying agents are added to the emulsion, such as flavoring agents (e.g. flavoring agents that confer fruit flavors, such as sour apple, peach, or other flavors, such as spearmint, peppermint, mint or cinnamon) and sweetening agents (e.g. sucralose). Other ingredients can be added, such as pH adjusting agents (e.g. acids, such as, but not limited to citric acid), which can be used to adjust the pH of the composition, typically to between at or about 2 and at or about 5, e.g. at or about 2 and at or about 3.5. Thus, the provided compositions typically have a pH of between at or about 2 and at or about 5, e.g. at or about 2 and at or about 3.5, such as 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9 or 5.

Before and/or after adding additional ingredients, the composition can be evaluated, such as by measuring the pH using an instrument such as those described herein. In one example, additional ingredients (e.g. pH adjusters) are added based on information obtained by evaluating the composition. The composition can be analyzed/evaluated to verify and/or determine other properties of the composition, for example, to verify that the composition contains the appropriate amounts of the active ingredients and other ingredients, to verify that microbial and heavy metal (e.g. arsenic, cadmium, mercury, lead and others) levels are within the acceptable range according to food/beverage standards. In one example, the acceptable microbial levels are not more than 1,000 cfu/g microbes (e.g. yeast, bacteria, mold and other microbes) and negativity for E. Coli and Salmonella; in another example, the acceptable heavy metal levels are not more than 10 ppm heavy metals, not more than 0.2 ppm lead and not more than 2 ppm arsenic. When a standard exists for a particular amount and/or property, the amount/property is verified by tests in accordance with U.S. Pharmacopeia (USP) and/or AOAC (Association of Analytical Communities) standards. Samples can be analyzed in accordance with these standards by sending a sample of the composition to a commercial testing facility, such as Eurofins U.S., Des Moines, Iowa or Advanced Botanical Consulting & Testing, Inc., Tustin, Calif., or other facility that performs tests in accordance with these standards.

For example, the amount of some active ingredients, such as caffeine anhydrous, chromium picolinate and Vitamin B12 typically is verified according to USP standards. The density and pH of the composition and the level of microbes, e.g. yeast, mold, E. coli and Salmonella, also typically are verified according to USP standards. The amount of fatty acids, e.g. CLA, can be verified according to AOAC standards, for example, by gas chromatography (GC), gas liquid chromatography (GLC) or other fatty acid profiling method. The levels of heavy metals, e.g. lead and arsenic, are tested using Inductively coupled plasma mass spectrometry (ICP-MS), by sending a sample of the composition for testing to a testing facility, such as Eurofins U.S., Des Moines, Iowa or Advanced Botanical Consulting & Testing, Inc., Tustin, Calif., or other facility. The amount of aminoalkane active ingredient, e.g. 2-amino-4-methylhexane HCl typically is verified by High-performance liquid chromatography (HPLC). Additionally, Fourier transform spectroscopy (FTIR) typically is used to obtain a fingerprint of the composition, to verify that no other compounds except the desired ingredients are present in the composition.

The composition can be purified, e.g. filtered, prior to use, using any purification means described herein or other purification means. Water can be added in the case of evaporation, to bring the composition up to the appropriate volume. HPLC, GC, GLC, FTIR and ICP-MS can be performed according to well-known methods (see, for example, Analytical Chemistry An Introduction 6th Ed., Douglas A. Skoog et al., (1994) Chapters 22 (FTIR) and 27 (GC/GLC, HPLC) and U.S. Pat. No. 6,265,717 (ICP-MS)).

After evaluation, purification, and/or addition of all the ingredients, the composition is packaged, for example, into large containers for storage or into containers for administration, such as bottles or ampoules(as described below). The composition can be transferred to the packaging containers using transfer means, such as transfer means described herein, including transfer pumps and fittings as described above or by manual transfer. In one example, the composition is packaged for storage in containers, such as totes, e.g. 275 gallon totes (such as the 275 Gallon Bottle with a Reconditioned CageTote Tank IBC, sold by Qualserv Enterprises, Inc. (i.e., see qualservcontainer.com), item # REN275), by transferring the mixture using a food grade hose (Sani-Tech® STHT-R-HD Braid-Reinforced Heavy Duty Silicone Hose), sold by Sani-Tech West. The tote typically is closed and sealed, e.g. tied, for example, with a cable tie.

h. Cleaning the Equipment

The equipment used in the provided methods is cleaned prior to, and typically after, use. For example, the methods include cleaning all the equipment in a sink and/or rinsing the vessels, e.g. tanks, and hose lines. The tanks are filled with hot water, washed with soap and water and rinsed and the water is discharged at pH 6-9. The pH of the water is adjusted using Soda ash, citric acid and/or H₃P0₄. The tanks then are sanitized with isopropyl alcohol (IPA) and let dry.

3. Analysis of Vasoconstrictor Activity

The provided compositions include active ingredients with vasoconstrictor activity (aminoalkanes and derivatives thereof and optionally other vasoconstrictive active ingredients); the vasoconstrictor activity typically is comparable to that of 2-amino-4-methylhexane or 2-amino-4-methylhexane HCl, for example, a particular amount (e.g. concentration) of 2-amino-4-methylhexane or 2-amino-4-methylhexane HCl.

For example, the compositions contain aminoalkanes and/or aminoalkane derivatives at a concentration having a vasoconstrictor activity equivalent to the vasoconstrictor activity of 2-amino-4-methylhexane HCl at a concentration of between at or about 3 mM and at or about 1000 mM, typically between at or about 3 mM and at or about 500 mM, e.g. between at or about 3 mM and at or about 250 mM, e.g. 5, 10, 15, 20, 25, 30, 35, 40, 45, 50, 55, 60, 65, 70, 75, 80, 85, 90, 95, 100, 101, 102, 103, 104, 105, 106, 107, 108, 109, 110, 120, 130, 140, 150, 160, 170, 180, 190, 200, 210, 220, 230, 240 or 250 mM or more, such as between at or about 5 mM and 150 mM, e.g. between 25 mM and 110 mM, e.g. at or about 107 mM; or at an amount that has a vasoconstrictor activity equivalent to the vasoconstrictor activity of between at or about 100 and 1000 micromoles of 2-amino-4-methylhexane HCl, e.g. at or about, 100, 110, 120, 130, 140, 150, 160, 170, 180, 190, 200, 225, 250, 275, 280, 290, 300, 310, 320, 330, 340, 350, 360, 370, 380, 390, 400, 425, 426, 427, 428, 429, 430, 450, 500, 600, 700, 800, 900 micromoles or more; or between at or about 15 g and 200 g, e.g. 15, 16, 17, 18, 19, 20, 21, 22, 23, 24, 25, 26, 27, 28, 29, 30, 40, 50, 55, 60, 65, 70, 75, 80, 85, 90, 95, 100, 125, 150 or 200 g, for example, at or about 65 g 2-amino-4-methylhexane HCl, for example, in a volume of between at or about 1 and 10 mL, typically between 2 and 5 mL, such as 4 mL.

Vasoconstrictor activity, including whether two compounds have equivalent vasoconstrictor activity and/or the amount of a test compound having equivalent vasoconstrictor activity to another compound, can be determined by assays known in the art, including assays that measure vasoconstrictor activity of vasoconstrictors such as epinephrine or adrenaline, such as those assays that have been used to measure the vasoconstrictor activity of aminoalkanes, e.g. 2-aminoalkanes (see Swanson and Chen, Journal of Pharmacology and Experimental Therapeutics, 88(1), 10-13 (1946), citing Swanson et al., Journal of Pharmacology and Experimental Therapeutics, 85(1), 70-73 (1945) and Swanson et al., Journal of Pharmacology and Experimental Therapeutics, 79(4), 329-333 (1943). Such assays include, for example, evaluation of vasoconstriction in animal subjects as described in Elliott, J. Physiol. 44: 374-409 (1912), where a test compound is injected intravenously into a pithed animal subject and blood pressure of the subject is measured. Animal subjects can include, but are not limited to, cats, dogs, guinea pigs, rabbits, primates, frogs, rats and mice. Blood pressure is measured before and after administration of the compound and vasoconstrictor activity of the test compound is determined as a function of the change in blood pressure.

In one variation, an animal (e.g. cat or dog) is pithed according to methods known in the art (e.g. anesthetized and its brain destroyed by insertion of a probe upward through the foramen magnum at the base of the skull). Artificial respiration is then induced following insertion of a tracheal cannula. After a short delay, a probe is inserted into the orbit and routed through the cranial cavity and down the spinal canal to about the fourth thoracic segment. After the blood pressure stabilizes, the circulatory system is ready for the assay. The test compound is injected into the external jugular vein at various doses and at various intervals. Blood pressure measurements are taken and plotted on a graph to determine the activity of the compound.

Generally, a rise in blood pressure is indicative of an active vasoconstriction compound. The vasoconstrictor activity typically is assessed by comparing change in blood pressure following administration of the test compound to change in blood pressure following administration (e.g. in the same animal), of a dose, and typically a range of doses, of a standard compound, such as epinephrine or adrenaline, or an aminoalkane, e.g. 2-amino-4-methylhexane or 2-amino-4-methylhexane HCl.

Alternatively, vasoconstriction activity can be measured using a rhinometric assay. In this type of assay, changes in the nasal cavity patency (i.e. degree of nasal cavity clearance or blockage) are evaluated in the presence of a test compound. Animal subjects for use in the rhinometric assay include, but are not limited to, rats, guinea pigs, rabbits, cats, dogs and pigs. Rhinometric assays include assays where nasal cavity geometry is measured, such as in acoustic rhinometry (see e.g., Rinder and Lundberg, Acta Physiol Scand 157: 233-244 (1996)). The assays also include those where nasal resistance values are derived from measurements of airflow and differential pressures between proximal and distal points within the nasal cavity (see e.g., Salem and Clemente, Arch Otolaryngology 96: 524-529 (1972) and McLeod et al, J Pharmacological and Toxicological Meth. 48: 153-159 (2002)). In one variation of the rhinometric assay, an allergen, such as ovalbumin, is administered to an animal subject, such as a guinea pig, in the presence or absence of a test compound, and changes in nasal patency are assessed based on measurements of forced airflow across the nasal cavity. Typically, the esophagus, mouth and one nostril are sealed in order to precisely direct the airflow generated from a tracheal cannula through the single open nasal passage. Pressure changes across the cavity due to the forced airflow can be measured using a physiological pressure transducer. Nasal pressure values can be converted to nasal resistance using the following formula: resistance=Δ pressure/flow. Activity of the test compound is determined as a function of the change in nasal resistance when the subject is challenged with an allergen. Generally, an active vasoconstrictor compound will decrease the degree of nasal resistance during an allergen challenge.

With the vasoconstrictor assays, the vasoconstrictor activity can be expressed by comparison to a standard compound (e.g. an epinephrine equivalent, see Swanson and Chen, Journal of Pharmacology and Experimental Therapeutics, 88(1), 10-13 (1946)). The values (e.g. change in blood pressure or nasal resistance) obtained in the assays with the test compounds are compared to values obtained, e.g. in the same animal subjects, with an amount, and typically a range of amounts, of a standard compound, such as one with a known vasoconstrictor activity, e.g. epinephrine, or a 2-aminoalkane, such as 2-amino-4-methylhexane or 2-amino-4-methylhexane HCl. In this example, the vasoconstrictor activity can be expressed as an equivalent of a known amount of the standard compound, such as an epinephrine equivalent, which is the amount of the standard compound that has equivalent activity to a given amount (e.g. 1 mg or 1 mole or 100 mM) of the test compound. For example, the average epinephrine equivalent can be listed for 1 mg of a plurality of different aminoalkanes, where the epinephrine equivalent is the amount of epinephrine that contains the equivalent vasoconstrictor activity to 1 mg of the aminoalkane (see, for example, Swanson and Chen, Journal of Pharmacology and Experimental Therapeutics, 88(1), 10-13 (1946)).

Thus, determining an amount of a first compound, such as an active ingredient (e.g. aminoalkane or biocompatible derivative), that has equivalent vasoconstrictor activity to a specified amount of a second compound (e.g. 2-aminoalkane-4-methylhexane HCl) can be done by comparing one or more amounts (e.g. mass, quantity or concentration) of the first compound to the specified amount (e.g. mass, quantity or concentration) of the second compound in a vasoconstrictor assay; or can be determined by first determining the standard compound equivalent (e.g. the epinephrine equivalent) of an amount of each compound in a vasoconstrictor assay, and then determining the amount of the first compound that is needed to equal the same standard compound (e.g. epinephrine) equivalent to the specific amount of the second compound (e.g. 2-amino-4-methylhexane HCl).

### D. COMBINATIONS, ARTICLES OF MANUFACTURE AND KITS

Provided herein are combinations containing the compositions, such as articles of manufacture and kits containing the compositions. For example, the compositions containing aminoalkane or aminoalkane derivative active ingredients can be packaged as articles of manufacture containing packaging material, an effective amount of the active ingredient (e.g. specified amount of vasoconstrictor activity), and, typically, a label that indicates that the composition and combinations are to be used for administration of the active ingredient, for example, to supplement the diet and/or to cause one or more biologic effects, such as sympathomimetic effects, stimulatory effects, vasoconstriction, decongestion (e.g. bronchial or nasal decongestion), increased energy, endurance, mood-enhancement, appetite suppression and/or weight loss.

Exemplary of the packaging material are containers, including single chamber and dual chamber containers. The containers include, but are not limited to, ampoules, such as ampoules made of soft plastic, such as low-density polyethylene (LDPE); tubes; bags; bottles; and syringes. The packaging materials further include and any other known packaging materials, such as pharmaceutical packaging materials, including, but not limited to, blister packs, bottles, tubes, inhalers, pumps, bags, vials, containers, syringes, needles, bottles, and any packaging material suitable for a selected formulation and intended mode of administration and treatment.

The compositions containing aminoalkane or aminoalkane derivative active ingredients also can be provided as kits. Kits can include a composition described herein and an item for administration. For example compositions can be supplied with a device for administration, such as a syringe, including a pre-filled syringe, an ampoule, an inhaler, a dosage cup, a dropper, or an applicator. The kit can, optionally, include instructions for application including dosages, dosing regimens and instructions for modes of administration.

Ampoules

Typically, the compositions are provided in containers, typically ampoules. The ampoules include ampoules made of soft-plastic, such as, but not limited to as low-density polyethylene (LDPE). Typically, the ampoules are twist-top ampoules, where the seal is broken by twisting the top of the ampoule. The ampoules contain ampoules for delivery of the low-volume compositions, e.g. ampoules having a total capacity (volume) of at or about, or less than at or about, 5000, 4000, 3000, 2000, 1000, 900, 800, 700, 600 mL, and typically less than at or about 500, 450, 400, 350, 300, 250, 200, 150, 100, 75, 50, 40, 30, 20, 19, 18, 17, 16, 15, 14, 13, 12, 11, 10, 9, 8, 7, 6, 5, 4, 3, 2, 1, or fewer, mL, such as ampoules having a 4.5 mL or 5 mL capacity. Exemplary of the ampoules are those available from Healthstar® Inc., Braintree, Mass. The compositions can be sent to a commercial packager, e.g. Healthstar® Inc., Braintree, Mass., for packaging into the containers, e.g. ampoules. The compositions typically are packaged in the ampoules for oral delivery, e.g. administration to a subject.

### E. METHODS FOR PROVIDING THE ACTIVE INGREDIENTS BY DELIVERY OF THE COMPOSITIONS

Also provided are methods for delivery of the active ingredients using the compositions, such as methods for providing an effective amount of the active ingredient to a subject by delivery of the compositions. For example, provided are methods for providing of an effective amount of an aminoalkane or derivative thereof, such as an amount effective to cause, improve or promote a biologic effect in a subject, for example, sympathomimetic effects, stimulatory effects, vasoconstriction, decongestion (e.g. bronchial or nasal decongestion), increased energy, endurance, mood-enhancement, appetite suppression and/or weight loss, or an amount necessary for preventing, curing, ameliorating, arresting or partially arresting a symptom of a disease or disorder, or the quantity and/or concentration desired by an individual for intake, such as daily intake, and/or nutritional supplementation, for example, an amount sufficient to enhance the nutritional, pharmaceutical, nutraceutical, health or energy property of a food, beverage, or other consumable.

The provided compositions typically are formulated for delivery via an oral route (e.g. ingestion of the composition by a subject), although other routes of administration are contemplated, such as any route for delivery of pharmaceuticals and nutraceuticals known to those of skill in the art, including parenteral administration, e.g. intramuscular, intravenous, intradermal, intralesional, intraperitoneal injection, epidural; nasal; oral; vaginal; rectal; topical; local; optic; inhalational; buccal (e.g., sublingual); and transdermal administration, or any route. Formulations suited for such routes are known to one of skill in the art.

When the composition is in an ampoule, delivery can be effected by breaking the seal on the ampoule, such as by twisting the twist-top of an ampoule, and then administering the contents of the ampoule to the subject, e.g. for oral ingestion, such as directly from the ampoule.

The following examples are included for illustrative purposes only and are not intended to limit the scope of the invention.

### F. EXAMPLES

### Example 1

### Production of a Palatable Low-Volume Composition Containing 2-amino-4-methylhexane HCl, Peach Flavoring Agent and Sucralose with a Scaled-Up Process

Table 2, below, set forth ingredients used to make a palatable low-volume composition containing 2-amino-4-methylhexane HCl (16.25 mg/mL) and a combination of taste-modifying agents that included a peach flavoring agent and sucralose. The composition was made according to the provided methods using a scaled-up process. The table indicates the amount (milligrams (mg)) of each ingredient that was contained per 4 mL serving of the composition and the percentage by weight and amount (g) of each ingredient per batch. The column labeled “phase” indicates to which phase each ingredient was added. For example, “water” indicates that a particular ingredient was added during production of the water phase, “oil” indicates the ingredient was added during production of the oil phase and “emulsion/flavor” indicates the ingredient was added during or after mixing of the water and oil phases, as described in further detail in this Example, below.

The CLA was CLA compound sold under the trade name Tonalin®, by Cognis Corporation; the caffeine anhydrous was Caffeine Anhydrous powder (white, crystalline powder), sold by Pacific Rainbow International Inc., City of Industry, Calif.; the Vitamin B12 was the pseudo Cyanocobalamin Vitamin B12 powder sold by Hebei Yuxing Bio-engineering Co., LTD, China; the chromium picolinate was Bio Chromium Picolinate, distributed by Stauber Performance Ingredients, Inc., Fullerton, Calif. (item #21243); the phosphatidylcholine was the phosphatidylcholine composition sold by Lipoid, LLC, under the name Alcolec PC95; the emulsion stabilizer was the SALADIZER®, available from TIC Gums, Inc. (Belcamp, Md.); the flavor was the peach flavoring agent sold by Mission Flavors and Fragrances, Inc., Foothill Ranch, Calif., catalog number PH-147; the sucralose was Trichlorosucrose (sucralose) sold by CHANGZHOU TIANHUA I&E CO LTD, NIUTANG TOWN, CHANGZHOU JIANGSU CHINA; the water was purified city water, purified as described below.

TABLE 2
Palatable Low-Volume Composition Containing 2-amino-4-
methylhexane HCl, peach flavoring agent and sucralose

| | | | Percent | |
|---|---|---|---|---|
| | mg/4 mL | | (by weight) of | |
| Ingredient | serving | Phase | composition | g/batch |
| Water | 3615.50 | Water | 90.388 | 1539693.215 |
| Potassium Sorbate | 1.600 | Water | 0.040 | 681.4 |
| (preservative) | | | | |
| Sodium Benzoate | 1.600 | Water | 0.040 | 681.4 |
| (preservative) | | | | |
| SALADIZER ® brand emulsion stabilizer | 6.600 | Water | 0.165 | 2810.7 |
| (blend of xanthan gum, guar gum and | | | | |
| sodium alginate) | | | | |
| Vitamin B12 | 1.000 | Water | 0.025 | 425.86 |
| Chromium Picolinate | 0.6 | Water | 0.015 | 255.515 |
| 2-Amino 4-Methylhexane HCl | 65.00 | Water | 1.625 | 27680.8350 |
| Caffeine Anhydrous | 80.0 | Water | 2.000 | 34068.720 |
| Conjugated Linoleic Acid (CLA) | 9.9 | Oil | 0.248 | 4216.004 |
| Phosphatidylcholine (co-surfactant) | 3.0 | Oil | 0.075 | 1277.577 |
| Tocopherol Polyethylene Glycol Succinate | 25.0 | Oil | 0.625 | 10646.475 |
| (TPGS; surfactant) | | | | |
| Sucralose (sweetening agent) | 57.5 | Emulsion/Flavor | 1.438 | 24486.893 |
| Citric Acid (pH adjuster) | 106.200 | Emulsion/Flavor | 2.655 | 45226.2258 |
| Peach flavoring agent (PH-147) | 26.500 | Emulsion/Flavor | 0.663 | 11285.2635 |
| Totals | 4000.000 | | 100.0000 | 1703436 |

The composition was made using the following scaled-up process:

Before adding to the appropriate phase, as described below, the correct amount (indicated in Table 2) of each ingredient was weighed out using either a Sartorius Basic Analytical Scale (Model BA110S), an OHAUS Scale (Model CS2000) or a Toledo Scale (Model GD13x/USA). Liquid ingredients were weighed in containers, while dry ingredients were weighed in bags. Water-jacket lines on water jacketed oil phase and water phase tanks were bled. The water-jacket switches then were turned on to maintain a pressure in the water jackets of between 20 and 40 PSI (pounds per square inch). If the pressure in the water jacket fell below 20 PSI during the method, the line was bled and checked for bubbles while purging the line.

### Production of the Water Phase:

The water phase was prepared in a water phase tank (an Overly 550 Gallon water jacketed tank (Model 10576501G), having a 550 gallon capacity), as follows:

A switch on a control panel was turned on, which simultaneously turned on a reverse osmosis pump and a UV sterilizer to purify the water. The appropriate amount of city water was purified by passing the water through the following purifiers, sequentially, in the following order: a carbon filter, an ion exchange purifier, a reverse osmosis purifier and a 100 micron end-point filter. The water (amount indicated in Table 2, above) finally was passed through a UV sterilizer before it was dumped into the water phase tank. The switch controlling the pump and UV sterilizer was turned off, and an Arde Barinco reversible homogenizer, Model no. CJ-4E Arde Barinco, Inc., Norwood, N.J.), mounted to the top of the water phase tank, was turned on, using the “forward” setting, at a speed of 500-1800 rpm.

Indicated amounts of the following ingredients then were added to the water phase tank, sequentially, in the following order: Potassium Sorbate, Sodium Benzoate, Vitamin B12 and Chromium Picolinate. The water phase then was heated to 600 using the water jacket on the water phase tank. After the water phase ingredients reached 60° C., the indicated amount of the SALADIZER® brand emulsion stabilizer (blend of xanthan gum, guar gum and sodium alginate) was added. The ingredients were mixed until fully dispersed in the water phase, using the homogenizer attached to the top of the water tank, using the “forward” setting at a speed of 500-1800 rpm. The indicated amount of the 2-Amino 4-Methylhexane HCl then was added (while the water phase was still at 60° C.), followed by the Caffeine Anhydrous. Mixing was continued until ingredients had dispersed and the water phase had reached 60° C., and until combining with the oil phase. Temperatures were measured with a pH and temperature meter (Hanna Instruments, model HI 8314).

### Production of the Oil Phase:

The oil phase was prepared in an oil phase tank (Royal 190 gallon water jacketed tank (Model 9977-5), having a 190 gallon capacity) as follows:

The indicated amounts of the Conjugated Linoleic Acid (CLA) and phosphatidylcholine were added sequentially, in that order, to the oil phase tank. The ingredients were heated to 60° C. until dissolved. The indicated amount of TPGS then was added to the oil phase at 60° C. and dissolved by mixing with a stainless steel paddle or spatula. Temperatures were measured with a pH and temperature meter (Hanna Instruments, model HI 8314).

Combining the Water and Oil Phases

Once the water phase and oil phase had been produced and were at 60° C., the homogenizer mounted at the top of the water phase tank was turned on at 1000-1500 RPM and the oil phase transferred to the water phase tank using transfer means, which included a transfer pump (Teel pump, model 2P377B, sold by Granger, Inc.), sanitary fittings, food grade transfer hose (sold by Sani-Tech West) positioned between openings in the two tanks, and ball valve(s). For the transfer, after turning on the homogenizer, ball valves were opened and the transfer pump turned on, effecting transfer of the oil phase liquid to the water phase tank via the transfer hose. As the phases were combined, mixing with the homogenizer continued until the phases had combined.

The indicated amounts of the taste-modifying agents (peach flavoring agent and sucralose) and the citric acid then were added to the tank. The ingredients were mixed and cooled by recirculation through a recirculating cooler (Model No. OC-1000 RO, sold by Turmoil, West Swanzey, N.H.), which was attached to the water phase tank, until the mixture reached between 30 and 35° C. Temperatures were measured with a pH and temperature meter (Hanna Instruments, model HI 8314). The pH of the mixture was measured with a pH and temperature meter (Hanna Instruments, model HI 8314), to confirm that it was between 2.00 and 2.40.

Analysis and Packaging

Other properties of the resulting composition also were analyzed to verify that it contained amounts of active and other ingredients and levels of microbiologicals and heavy metals that were in accordance with standards, as described herein. USP standard testing by Eurofins U.S., Des Moines, Iowa verified that the composition contained not more than 1,000 cfu/g microbes, was negative for E. Coli and Salmonella; ICP/MS testing by Eurofins U.S. verified that the composition contained not more than 10 ppm heavy metals, not more than 0.2 ppm lead and not more than 2 ppm arsenic. The amounts of Vitamin B12, Caffeine Anhydrous and Chromium picolinate in the composition were verified by tests in accordance with USP standards, by Eurofins U.S. The amount of CLA, was verified according to AOAC standards by gas chromatography (GC). The amount of 2-amino-4-methylhexane HCl was verified by High-performance liquid chromatography (HPLC). Additionally, Fourier transform spectroscopy (FTIR) can be used to obtain a fingerprint of the composition, to verify that no other compounds except the desired ingredients are present in the composition.

The composition then was packaged into 275 gallon totes (275 Gallon Bottle with a Reconditioned CageTote Tank IBC, sold by Qualserv Enterprises, Inc. (see, e.g., qualservcontainer.com), item # REN275) by transferring the mixture using a food grade hose (Sani-Tech® STHT-R-HD Braid-Reinforced Heavy Duty Silicone Hose), sold by Sani-Tech West. The tote was closed and sealed by tying with a cable tie.

### Example 2

### Production of Palatable Low-Volume Compositions Containing 2-amino-4-methylhexane HCl, and Various Taste-Modifying Agents, with a Bench-Top Process

Tables 3-7, below, set forth ingredients used to make five palatable low-volume liquid compositions, each composition containing 2-amino-4-methylhexane HCl (16.25 mg/mL), according to the provided methods, using a bench-top process as described in this example, below. Any of these exemplary compositions alternatively can be made using the scaled-up processes as provided herein.

Each table indicates the amount (milligrams (mg)) of each ingredient that was contained per 4 mL serving of the composition and the percentage by weight and amount (g) of each ingredient per batch. The column labeled “phase” indicates to which phase each ingredient was added. For example, “water” indicates that a particular ingredient was added during production of the water phase, “oil” indicates the ingredient was added during production of the oil phase and “emulsion/flavor” indicates the ingredient was added during or after mixing of the water and oil phases. The flavoring agents used in the compositions set forth in Tables 3-7 were a peppermint flavoring agent, a cinnamon flavoring agent, spearmint flavoring agent, sour apple flavoring agent, and mint flavoring agent, purchased from Mission Flavors and Fragrances, Inc., Foothill Ranch, Calif. (catalog numbers MI-104, L-9806, MI-110, AP-138 and L-12389, respectively).

In each of the compositions set forth in Tables 3-7, the CLA was CLA compound sold under the trade name Tonalin®, by Cognis Corporation; the caffeine anhydrous was Caffeine Anhydrous powder (white, crystalline powder), sold by Pacific Rainbow International Inc., City of Industry, Calif.; the phosphatidylcholine was the phosphatidylcholine composition sold by Lipoid, LLC, under the name Alcolec PC95; the emulsion stabilizer was the SALADIZER®, available from TIC Gums, Inc. (Belcamp, Md.); the sucralose was Trichlorosucrose (sucralose) sold by CHANGZHOU TIANHUA I&E CO LTD, NIUTANG TOWN, CHANGZHOU JIANGSU CHINA; the water was purified city water, purified as described below.

TABLE 3
Palatable low-volume composition containing 2-amino-4-
methylhexane HCl, peppermint flavoring agent and sucralose

| | | | Percent | |
|---|---|---|---|---|
| | mg/4 mL | | (by weight) of | |
| Ingredient | serving | Phase | composition | g/batch |
| Water | 3598.60 | Water | 89.965 | 449.825 |
| Potassium Sorbate (preservative) | 1.600 | Water | 0.040 | 0.2 |
| Sodium Benzoate (preservative) | 1.600 | Water | 0.040 | 0.2 |
| SALADIZER ® brand emulsion stabilizer | 6.600 | Water | 0.165 | 0.8 |
| (blend of xanthan gum, guar gum and | | | | |
| sodium alginate) | | | | |
| 2-Amino 4-Methyl Hexane HCl | 65.00 | Water | 1.625 | 8.1250 |
| Caffeine Anhydrous | 80.0 | Water | 2.000 | 10.000 |
| Conjugated Linoleic Acid (CLA) | 9.9 | Oil | 0.248 | 1.238 |
| Phosphatidylcholine (co-surfactant) | 3.0 | Oil | 0.075 | 0.375 |
| Tocopherol Polyethylene Glycol Succinate | 30.0 | Oil | 0.750 | 3.750 |
| (TPGS; surfactant) | | | | |
| Sucralose (sweetening agent) | 57.5 | Emulsion/Flavor | 1.438 | 7.188 |
| Citric Acid (pH adjusting agent) | 106.200 | Emulsion/Flavor | 2.655 | 13.2750 |
| Peppermint flavoring agent (MI-104) | 40.000 | Emulsion/Flavor | 1.000 | 5.0000 |
| Totals | 4000.00 | | 100.000 | 500 |

TABLE 4
Palatable low-volume composition containing 2-amino-4-
methylhexane HCl, cinnamon flavoring agent and sucralose

| | | | Percent | |
|---|---|---|---|---|
| | mg/4 mL | | (by weight) of | |
| Ingredient | serving | Phase | composition | g/batch |
| Water | 3598.60 | Water | 89.965 | 449.825 |
| Potassium Sorbate (preservative) | 1.600 | Water | 0.040 | 0.2 |
| Sodium Benzoate (preservative) | 1.600 | Water | 0.040 | 0.2 |
| SALADIZER ® brand emulsion stabilizer | 6.600 | Water | 0.165 | 0.8 |
| (blend of xanthan gum, guar gum and | | | | |
| sodium alginate) | | | | |
| 2-Amino 4-Methyl Hexane HCl | 65.00 | Water | 1.625 | 8.1250 |
| Caffeine Anhydrous | 80.0 | Water | 2.000 | 10.000 |
| Conjugated Linoleic Acid (CLA) | 9.9 | Oil | 0.248 | 1.238 |
| Phosphatidylcholine (co-surfactant) | 3.0 | Oil | 0.075 | 0.375 |
| Tocopherol Polyethylene Glycol Succinate | 30.0 | Oil | 0.750 | 3.750 |
| (TPGS; surfactant) | | | | |
| Sucralose (sweetening agent) | 57.5 | Emulsion/Flavor | 1.438 | 7.188 |
| Citric Acid (pH adjusting agent) | 106.200 | Emulsion/Flavor | 2.655 | 13.2750 |
| Cinnamon flavoring agent (L-9806) | 40.000 | Emulsion/Flavor | 1.000 | 5.0000 |
| Totals | 4000.00 | | 100.000 | 500 |

TABLE 5
Palatable low-volume composition containing 2-amino-4-
methylhexane HCl, spearmint flavoring agent and sucralose

| | | | Percent | |
|---|---|---|---|---|
| | mg/4 mL | | (by weight) of | |
| Ingredient | serving | Phase | composition | g/batch |
| Water | 3578.60 | Water | 89.465 | 447.325 |
| Potassium Sorbate (preservative) | 1.600 | Water | 0.040 | 0.2 |
| Sodium Benzoate (preservative) | 1.600 | Water | 0.040 | 0.2 |
| SALADIZER ® brand emulsion stabilizer | 6.600 | Water | 0.165 | 0.8 |
| (blend of xanthan gum, guar gum and | | | | |
| sodium alginate) | | | | |
| 2-Amino 4-Methyl Hexane HCl | 65.00 | Water | 1.625 | 8.1250 |
| Caffeine Anhydrous | 80.0 | Water | 2.000 | 10.000 |
| Conjugated Linoleic Acid (CLA) | 9.9 | Oil | 0.248 | 1.238 |
| Phosphatidylcholine (co-surfactant) | 3.0 | Oil | 0.075 | 0.375 |
| Tocopherol Polyethylene Glycol Succinate | 30.0 | Oil | 0.750 | 3.750 |
| (TPGS; surfactant) | | | | |
| Sucralose (sweetening agent) | 57.5 | Emulsion/Flavor | 1.438 | 7.188 |
| Citric Acid (pH adjusting agent) | 106.200 | Emulsion/Flavor | 2.655 | 13.2750 |
| Spearmint flavoring agent (MI-110) | 60.000 | Emulsion/Flavor | 1.500 | 7.5000 |
| Totals | 4000.00 | | 100.000 | 500 |

TABLE 6
Palatable low-volume composition containing 2-amino-4-
methylhexane HCl, sour apple flavoring agent and sucralose

| | | | Percent | |
|---|---|---|---|---|
| | mg/4 mL | | (by weight) of | |
| Ingredient | serving | Phase | composition | g/batch |
| Water | 3620.60 | Water | 90.515 | 452.575 |
| Potassium Sorbate (preservative) | 1.600 | Water | 0.040 | 0.2 |
| Sodium Benzoate (preservative) | 1.600 | Water | 0.040 | 0.2 |
| SALADIZER ® brand emulsion stabilizer | 6.600 | Water | 0.165 | 0.8 |
| (blend of xanthan gum, guar gum and | | | | |
| sodium alginate) | | | | |
| 2-Amino 4-Methyl Hexane HCl | 65.00 | Water | 1.625 | 8.1250 |
| Caffeine Anhydrous | 80.0 | Water | 2.000 | 10.000 |
| Conjugated Linoleic Acid (CLA) | 9.9 | Oil | 0.248 | 1.238 |
| Phosphatidylcholine (co-surfactant) | 3.0 | Oil | 0.075 | 0.375 |
| Tocopherol Polyethylene Glycol Succinate | 30.0 | Oil | 0.750 | 3.750 |
| (TPGS; surfactant) | | | | |
| Sucralose (sweetening agent) | 57.5 | Emulsion/Flavor | 1.438 | 7.188 |
| Citric Acid (pH adjusting agent) | 106.200 | Emulsion/Flavor | 2.655 | 13.2750 |
| Sour apple flavoring agent (AP-138) | 18.000 | Emulsion/Flavor | 0.450 | 2.2500 |
| Totals | 4000.00 | | 100.000 | 500 |

TABLE 7
Palatable low-volume composition containing 2-amino-4-methylhexane
HCl, Mint flavoring agent and sucralose

| | | | Percent | |
|---|---|---|---|---|
| | mg/4 mL | | (by weight) of | |
| Ingredient | serving | Phase | composition | g/batch |
| Water | 3674.27 | Water | 91.857 | 229.641875 |
| Potassium Sorbate (preservative) | 1.600 | Water | 0.040 | 0.1 |
| Sodium Benzoate (preservative) | 1.600 | Water | 0.040 | 0.1 |
| SALADIZER ® brand emulsion stabilizer | 6.600 | Water | 0.165 | 0.4 |
| (blend of xanthan gum, guar gum and | | | | |
| sodium alginate) | | | | |
| 2-Amino 4-Methyl Hexane HCl | 65.00 | Water | 1.625 | 4.0625 |
| Caffeine Anhydrous | 80.0 | Water | 2.000 | 5.000 |
| Conjugated Linoleic Acid (CLA) | 9.9 | Oil | 0.248 | 0.619 |
| Phosphatidylcholine (co-surfactant) | 3.0 | Oil | 0.075 | 0.188 |
| Tocopherol Polyethylene Glycol Succinate | 50.0 | Oil | 1.250 | 3.125 |
| (TPGS; surfactant) | | | | |
| Sucralose (sweetening agent) | 57.5 | Emulsion/Flavor | 1.438 | 3.594 |
| Citric Acid (pH adjusting agent) | 6.800 | Emulsion/Flavor | 0.170 | 0.4250 |
| Mint flavoring agent (L-12389) | 43.725 | Emulsion/Flavor | 1.093 | 2.7328 |
| Totals | 4000.00 | | 100.000 | 250 |

Each composition was made using the following bench-top process:

Before adding to the appropriate phase, as described below, the correct amount (indicated in Tables 3-7) of each ingredient was weighed out using either a Sartorius Basic Analytical Scale (Model BA110S), an OHAUS Scale (Model CS2000) or a Toledo Scale (Model GD13x/USA). Liquid ingredients were weighed in containers, while dry ingredients were weighed in bags.

### Production of the Water Phase:

For each of the compositions in Tables 3-7, the water phase was prepared in a water phase vessel, which was a Pyrex® beaker, as follows:

A switch on a control panel was turned on, which simultaneously turned on a reverse osmosis pump and a UV sterilizer to purify the water. The appropriate amount of city water was purified by passing the water through the following purifiers, sequentially, in the following order: a carbon filter, an ion exchange purifier, a reverse osmosis purifier and a 100 micron end-point filter. The water (amount indicated in Tables 3-7, above) finally was passed through a UV sterilizer before it was added into the water phase vessel. The switch controlling the pump and UV sterilizer was turned off, and an Arde Barinco reversible homogenizer, Model no. CJ-4E Arde Barinco, Inc., Norwood, N.J.), in the water phase vessel, was turned on, using the “forward” setting, at a speed of 500-1800 rpm.

The indicated amounts of the following ingredients were added to the water phase vessel, sequentially, in the following order: potassium sorbate and sodium benzoate. The water phase then was heated to 60° using a heating apparatus, which was a hot plate (Thermolyne hot Plate Model # SP46615, Barnstead International, Dubuque, Iowa). After the water phase ingredients reached 60° C., the indicated amount of the SALADIZER® brand emulsion stabilizer (blend of xanthan gum, guar gum and sodium alginate) was added. The ingredients were mixed until fully dispersed in the water phase, using an Arde Barinco reversible homogenizer, Model no. CJ-4E Arde Barinco, Inc., Norwood, N.J.) using the “forward” setting, with a speed of 500-1800 rpm.

The indicated amount of 2-Amino 4-Methylhexane HCl then was added while the water phase temperature was maintained at 60° C., followed by the Caffeine anhydrous. Mixing with the homogenizer was continued until ingredients had dispersed and the water phase had reached 60° C., and until combining with the oil phase. Temperatures were measured with a pH and temperature meter (Hanna Instruments, model HI 8314) and/or a temperature probe (Model # DPP400W, Cooper-Atkins).

Production of the Oil Phase:

For each of the compositions listed in Tables 3-7, the oil phase was prepared in an oil phase vessel, which was a Pyrex® beaker, as follows:

The indicated amounts of the Conjugated Linoleic Acid (CLA) and phosphatidylcholine were added sequentially, in that order, to the oil phase vessel. The ingredients were heated to 60° C., using a heating apparatus, which was a hot plate (Thermolyne hot Plate Model # SP46615, Barnstead International, Dubuque, Iowa), until dissolved. The indicated amount of TPGS then was added to the oil phase at 60° C. and dissolved by mixing with a stainless steel paddle or a spatula. Temperatures were measured with a pH and temperature meter (Hanna Instruments, model HI 8314) and/or a temperature probe (Model # DPP400W, Cooper-Atkins).

Combining the Water and Oil Phases

Once the water phase and oil phase had been produced and were at 60° C., a reversible homogenizer (Arde Barinco, Inc.; Model CJ-4E) in the water phase vessel was turned on at 1000-1500 RPM and the oil phase transferred to the water phase tank using manual transfer means, by pouring the oil phase from the oil phase vessel into the water phase vessel. As the phases were combined, mixing with the homogenizer continued until the phases had combined.

The indicated amounts of the taste-modifying agents (flavoring agents (peppermint, cinnamon, spearmint, sour apple or mint flavoring agents) and sucralose) and citric acid then were added to the vessel. The ingredients were mixed and cooled. The cooling was effected by placing the water phase vessel (beaker), containing the mixture emulsion, in a water bath, until the mixture reached between 30 and 35° C. Temperatures were measured with a pH and temperature meter (Hanna Instruments, model HI 8314) and/or a temperature probe (Model # DPP400W, Cooper-Atkins). The pH of the mixture was measured with a pH and temperature meter (Hanna Instruments, model HI 8314), to confirm that it was between 2.00 and 2.40. The compositions set forth in Tables 3-7 can be analyzed and packaged according to the methods herein.

### Example 3

### Production of a Palatable Low-Volume Composition Containing 2-amino-4-methylhexane HCl, Mint Flavoring Agent, and Sucralose with a Bench-Top Process

Table 8, below, set forth ingredients used to make a palatable low-volume liquid composition containing 2-amino-4-methylhexane HCl (16.25 mg/mL), according to the provided methods, using a bench-top process as described in this example, below. This exemplary composition alternatively can be made using the scaled-up processes as provided herein.

The table indicates the amount (milligrams (mg)) of each ingredient that was contained per 4 mL serving of the composition and the percentage by weight and amount (g) of each ingredient per batch. The column labeled “phase” indicates to which phase each ingredient was added. For example, “water” indicates that a particular ingredient was added during production of the water phase, “oil” indicates the ingredient was added during production of the oil phase and “emulsion/flavor” indicates the ingredient was added during or after mixing of the water and oil phases.

In the composition set forth in Table 8, the CLA was CLA compound sold under the trade name Tonalin®, by Cognis Corporation; the caffeine anhydrous was Caffeine Anhydrous powder (white, crystalline powder), sold by Pacific Rainbow International Inc., City of Industry, Calif.; the L-taurine was Taurine, sold by DNP International Co., Inc., Santa Fe Springs, Calif., or Taurine, manufactured by Qianjiang Yongan Pharmaceutical Co., Ltd., distributed by Stauber Performance Ingredients, Inc., Fullerton, Calif., catalog number 21195; the alpha lipoic acid was Alpha Lipoic Acid, sold by NutriChem Resources Company, Walnut, Calif., or Alpha Lipoic Acid, sold by Zhejiang Medicines & Health Products Import & Export Co., Ltd, Hangzhou, China; the phosphatidylcholine was the phosphatidylcholine composition sold by Lipoid, LLC, under the name Alcolec PC95; the emulsion stabilizer was the SALADIZER®, available from TIC Gums, Inc. (Belcamp, Md.); the flavor was a mint flavoring agent sold by Mission Flavors and Fragrances, Inc., Foothill Ranch, Calif., catalog number L-12389; the sucralose was Trichlorosucrose (sucralose) sold by CHANGZHOU TIANHUA I&E CO LTD, NIUTANG TOWN, CHANGZHOU JIANGSU CHINA; the water was purified city water, purified as described below.

TABLE 8
Palatable low-volume composition containing 2-amino-4-methylhexane
HCl, Mint flavoring agent and sucralose

| | | | Percent | |
|---|---|---|---|---|
| | mg/4 mL | | (by weight) of | |
| Ingredient | serving | Phase | composition | g/batch |
| Water | 3653.27 | Water | 91.332 | 913.3175 |
| Potassium Sorbate (preservative) | 1.600 | Water | 0.040 | 0.4 |
| Sodium Benzoate (preservative) | 1.600 | Water | 0.040 | 0.4 |
| SALADIZER ® brand emulsion stabilizer | 6.600 | Water | 0.165 | 1.7 |
| (blend of xanthan gum, guar gum and | | | | |
| sodium alginate) | | | | |
| 2-Amino 4-Methyl Hexane HCl | 65.00 | Water | 1.625 | 16.2500 |
| Caffeine Anhydrous | 80.0 | Water | 2.000 | 20.000 |
| L-Taurine | 10.0 | Water | 0.250 | 2.500 |
| Conjugated Linoleic Acid (CLA) | 9.9 | Oil | 0.248 | 2.475 |
| Phosphatidylcholine (co-surfactant) | 3.0 | Oil | 0.075 | 0.750 |
| Tocopherol Polyethylene Glycol Succinate | 60.0 | Oil | 1.500 | 15.000 |
| (TPGS; surfactant) | | | | |
| Alpha Lipoic Acid | 1.0 | Oil | 0.025 | 0.250 |
| Sucralose (sweetening agent) | 57.5 | Emulsion/Flavor | 1.438 | 14.375 |
| Citric Acid (pH adjusting agent) | 6.800 | Emulsion/Flavor | 0.170 | 1.7000 |
| Mint flavoring agent (L-12389) | 43.725 | Emulsion/Flavor | 1.093 | 10.9313 |
| Totals | 4000.00 | | 100.000 | 1000 |

The composition was made using the following bench-top process:

Before adding to the appropriate phase, as described below, the correct amount (indicated in Table 8) of each ingredient was weighed out using either a Sartorius Basic Analytical Scale (Model BA110S), an OHAUS Scale (Model CS2000) or a Toledo Scale (Model GD13x/USA). Liquid ingredients were weighed in containers, while dry ingredients were weighed in bags.

### Production of the Water Phase:

For the composition in Table 8, the water phase was prepared in a water phase vessel, which was a Pyrex® beaker, as follows:

A switch on a control panel was turned on, which simultaneously turned on a reverse osmosis pump and a UV sterilizer to purify the water. The appropriate amount of city water was purified by passing the water through the following purifiers, sequentially, in the following order: a carbon filter, an ion exchange purifier, a reverse osmosis purifier and a 100 micron end-point filter. The water (amount indicated in Table 8, above) finally was passed through a UV sterilizer before it was added into the water phase vessel. The switch controlling the pump and UV sterilizer was turned off, and an Arde Barinco reversible homogenizer, Model no. CJ-4E Arde Barinco, Inc., Norwood, N.J.), in the water phase vessel, was turned on, using the “forward” setting, at a speed of 500-1800 rpm.

Potassium Sorbate was added at the indicated amount was added to the water phase vessel. The water phase then was heated to 60° using a heating apparatus, which was a hot plate (Thermolyne hot Plate Model # SP46615, Barnstead International, Dubuque, Iowa). After the water phase ingredients reached 60° C., the indicated amount of the SALADIZER® brand emulsion stabilizer (blend of xanthan gum, guar gum and sodium alginate) was added. The ingredients were mixed until fully dispersed in the water phase, using an Arde Barinco reversible homogenizer, Model no. CJ-4E Arde Barinco, Inc., Norwood, N.J.) using the “forward” setting, with a speed of 500-1800 rpm.

The indicated amounts of the following ingredients were added to the water phase vessel while the water phase temperature was maintained at 60° C., sequentially, in the following order: Caffeine anhydrous; Sodium Benzoate; Vitamin B12; Chromium Picolinate; 2-Amino 4-Methylhexane HCl; and L-Taurine. Mixing with a Lightnin water phase mixer was continued until ingredients had dispersed and until combining with the oil phase. Temperatures were measured with a pH and temperature meter (Hanna Instruments, model HI 8314) and/or a temperature probe (Model # DPP400W, Cooper-Atkins).

Production of the Oil Phase:

For the composition listed in Table 8, the oil phase was prepared in an oil phase vessel, which was a Pyrex® beaker, as follows:

The indicated amounts of the Conjugated Linoleic Acid (CLA) and phosphatidylcholine were added sequentially, in that order, to the oil phase vessel. The ingredients were heated to 60° C., using a heating apparatus, which was a hot plate (Thermolyne hot Plate Model # SP46615, Barnstead International, Dubuque, Iowa), until dissolved. The indicated amount of TPGS then was added to the oil phase at 60° C. and dissolved by mixing with a stainless steel paddle or a spatula. The indicated amount of Alpha Lipoic Acid was then added to the oil phase at 60° C. with continued mixing. Temperatures were measured with a pH and temperature meter (Hanna Instruments, model HI 8314) and/or a temperature probe (Model # DPP400W, Cooper-Atkins).

Combining the Water and Oil Phases

Once the water phase and oil phase had been produced and were at 60° C., a reversible homogenizer (Arde Barinco, Inc.; Model CJ-4E) in the water phase vessel was turned on at 1000-1500 RPM and the oil phase transferred to the water phase tank using manual transfer means, by pouring the oil phase from the oil phase vessel into the water phase vessel. As the phases were combined, mixing with the homogenizer continued until the phases had combined.

The indicated amounts of sucralose and citric acid then were added to the vessel. The ingredients were mixed and cooled. The cooling was effected by recirculating through a chiller until the mixture reached between 30 and 35° C. The mint flavoring was the added with mixing. Temperatures were measured with a pH and temperature meter (Hanna Instruments, model HI 8314) and/or a temperature probe (Model # DPP400W, Cooper-Atkins). The pH of the mixture was measured with a pH and temperature meter (Hanna Instruments, model HI 8314), to confirm that it was between 2.00 and 2.40. The composition set forth in Table 8 can be analyzed and packaged according to the methods herein.

Since modifications will be apparent to those of skill in this art, it is intended that this invention be limited only by the scope of the appended claims.

1. A palatable liquid composition, comprising:
(a) an aminoalkane of formula I: 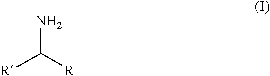
or a biocompatible derivative thereof, wherein:
R is an alkyl containing from 2 to 20 carbons; and
R′ is a hydrogen or an alkyl containing from 1-20 carbons;
the aminoalkane or derivative thereof has vasoconstrictor activity; and
the concentration of the aminoalkane or derivative thereof is between at or about 3 mM and at or about 500 mM;
(b) a surfactant in an amount between at or about 0.1% and at or about 25%, by weight, of the composition; and
(c) a taste-modifying agent, in an amount sufficient to improve or enhance the palatability of the composition compared to the absence of the agent.

2. The composition of claim 1, wherein the concentration of the aminoalkane or derivative thereof is between at or about 40 mM and at or about 250 mM or is between at or about 100 mM and at or about 110 mM.

3. The composition of claim 1, that is formulated as an emulsion.

4. The composition of claim 1, wherein the surfactant is a PEG-derivative of Vitamin E.

5. The composition of claim 1, wherein the aminoalkane is a 2-aminoalkane.

6. The composition of claim 1, wherein the aminoalkane contains between 4 and 9, or between 6 and 9, carbon atoms.

7. The composition of claim 1, wherein the aminoalkane contains a methyl substitution.

8. The composition of claim 7, wherein the aminoalkane is a 2-aminoalkane and contains a methyl substitution on the fourth carbon of the carbon chain.

9. The composition of claim 8, wherein the aminoalkane is 2-amino-4-methylhexane.

10. The composition of claim 1, wherein the aminoalkane or biocompatible derivative thereof is selected from among acid addition salts, aldehyde derivatives, amide derivatives, acid derivatives, ester derivatives and carbonate derivatives.

11. The composition of claim 10, wherein the aminoalkane or biocompatible derivative thereof is an acid addition salt selected from among acetic acid addition salts, hydrobromic acid addition salts, sulfuric acid addition salts, maleic acid addition salts, propionic acid addition salts, malonic acid addition salts and hydrochloric acid addition salts.

12. The composition of claim 11, wherein:
the aminoalkane or biocompatible derivative thereof is 2-amino-4-methylhexane HCl.

13. The composition of claim 1, wherein:
the aminoalkane or biocompatible derivative thereof is an acid addition salt, aldehyde derivative or carbonate derivative of 2-amino-4-methylhexane.

14. The composition of claim 1, wherein the taste-modifying agent is in the composition at an amount between at or about 0.1% and at or about 25%, or between at or about 0.45% and at or about 3%, by weight, of the composition.

15. The composition of claim 1, wherein the taste-modifying agent comprises a flavoring agent.

16. The composition of claim 15, wherein the flavoring agent is in the composition at an amount between at or about 0.1% and at or about 25%, or between at or about 0.45% and at or about 1.5%, by weight, of the composition.

17. The composition of claim 15, wherein the flavoring agent confers a flavor selected from among fruit flavors, cinnamon flavors and mint flavors.

18. The composition of claim 1, wherein the taste-modifying agent comprises sugar or a sugar substitute.

19. The composition of claim 18, wherein the sugar or sugar substitute is in the composition at an amount between at or about 0.1% and at or about 25%, by weight, of the composition, or between at or about 1.4% and at or about 25%, by weight.

20. The composition of claim 18, wherein the taste-modifying agent comprises a sugar substitute selected from among aspartame, saccharin, sucralose, neotame, and acesulfame potassium.

21. The composition of claim 1, further comprising a preservative, in an amount sufficient to preserve the composition compared to the absence of the preservative.

22. The composition of claim 21, wherein the preservative is selected from among any one or more of potassium sorbate and sodium benzoate.

23. The composition of claim 1, further comprising one or more additional active ingredients.

24. The composition of claim 23, wherein the one or more additional active ingredients are selected from any one or more of nutritional supplements, vitamins, minerals, fatty acids, amino acids, and weight-loss compounds.

25. The composition of claim 24, wherein the one or more additional active ingredients comprises any one or more of caffeine, Vitamin B12, chromium picolinate, conjugated linoleic acid (CLA), L-taurine, and alpha lipoic acid.

26. The composition of claim 4, wherein the surfactant is a tocopherol polyethylene glycol diester (TPGD).

27. The composition of claim 26, wherein the surfactant E is selected from among tocopherol polyethylene glycol succinate (TPGS), tocopherol sebacate polyethylene glycol, tocopherol dodecanodioate polyethylene glycol, tocopherol suberate polyethylene glycol, tocopherol azelaate polyethylene glycol, tocopherol citraconate polyethylene glycol, tocopherol methylcitraconate polyethylene glycol, tocopherol itaconate polyethylene glycol, tocopherol maleate polyethylene glycol, tocopherol glutarate polyethylene glycol, tocopherol glutaconate polyethylene glycol, tocopherol phthalate polyethylene glycol, a TPGS analog or a TPGS homolog.

28. The composition of claim 27, wherein the surfactant is a TPGS-1000 or d-α TPGS.

29. The composition of claim 27, wherein the surfactant is TPGS homolog that differs from a TPGS parent compound by one or more methylene unit(s).

30. The composition of claim 26, wherein the PEG moiety in the surfactant is selected from among any one or more of: methylated PEG (m-PEG), PEG-OH, PEG-NHS, PEG-aldehyde, PEG-SH, PEG-NH₂, PEG-CO₂H and branched PEGs.

31. The composition of claim 1, wherein the amount of the surfactant in the composition is between at or about 0.1% and at or about 1%, by weight, or between at or about 0.625% and at or about 0.75%, by weight, of the composition.

32. The composition of claim 1, further comprising a co-surfactant.

33. The composition of claim 32, wherein the co-surfactant contains a phospholipid.

34. The composition of claim 33, wherein the phospholipid comprises phosphatidylcholine.

35. The composition of claim 32, wherein the co-surfactant is in the composition at an amount between at or about 0.01% and at or about 25%, by weight, or at an amount of at or about 0.075%, by weight, of the composition.

36. The composition of claim 1, further comprising an additional component that stabilizes an emulsion in an amount sufficient to stabilize the composition compared to the absence of the emulsion stabilizer.

37. The composition of claim 36, wherein the emulsion stabilizer is in the composition at an amount between at or about 0.01% and at or about 25%, by weight, or between at or about 0.1% and at or about 0.2%, by weight, of the composition, or is at or about 0.165%, by weight, of the composition.

38. The composition of claim 36, wherein the emulsion stabilizer contains a blend of gums selected from any one or more of guar gum, xanthan gum and sodium alginate.

39. The composition of claim 1, comprising water at an amount between at or about 25% and at or about 95%, by weight, or between at or about 25% and at or about 92%, by weight, or between at or about 80% and at or about 92%, by weight, of the composition.

40. The composition of claim 39, wherein the amount of water is at or about 89%, 90%, 91% or 92%, by weight, of the composition.

41. The composition of claim 1, wherein:
the aminoalkane or derivative thereof is 2-amino-4-methylhexane HCl;
the taste-modifying agents include sucralose and a peach flavoring agent; and
the composition further comprises: caffeine, conjugated linoleic acid, phosphatidylcholine, a TPGS surfactant, an emulsion stabilizer comprising: guar gum, xanthan gum and sodium alginate, at an amount sufficient to stabilize the composition compared to the absence of the emulsion stabilizer, citric acid, at an amount sufficient to change the pH compared to the composition without the citric acid, water, and potassium sorbate and sodium benzoate, at a combined amount sufficient to preserve the composition compared to the absence of the potassium sorbate and sodium benzoate.

42. The composition of claim 1, wherein:
the aminoalkane or derivative thereof is 2-amino-4-methylhexane HCl;
the taste-modifying agents include sucralose and a peach flavoring agent; and
the composition further contains caffeine, conjugated linoleic acid, Vitamin B12, chromium picolinate, phosphatidylcholine, a TPGS surfactant, an emulsion stabilizer comprising: guar gum, xanthan gum and sodium alginate, at an amount sufficient to stabilize the composition compared to the absence of the emulsion stabilizer, citric acid, at an amount sufficient to lower the pH compared to the composition without the citric acid, water, and potassium sorbate and sodium benzoate, at a combined amount sufficient to preserve the composition compared to the absence of the potassium sorbate and sodium benzoate.

43. The composition of claim 41, wherein:
the 2-amino-4-methylhexane is in the composition at a concentration of between at or about 30 mM and at or about 200 mM;
the flavoring agent is a peach flavoring agent at an amount between at or about 0.45% and at or about 1.5%, by weight, of the composition;
the sucralose is in the composition in an amount of at or about 1.44%, by weight, of the composition;
the caffeine is in the composition in an amount of at or about 2%, by weight, of the composition;
the conjugated linoleic acid (CLA) is in the composition in an amount of at or about 0.25%, by weight, of the composition;
the phosphatidylcholine is in the composition in an amount of at or about 0.075%, by weight, of the composition;
the TPGS surfactant is in the composition in an amount between at or about 0.63% and at or about 1.25%, by weight, of the composition;
the emulsion stabilizer is in the composition at an amount of at or about 0.165%, by weight, of the composition;
the citric acid is in the composition in an amount of at or about 2.66%, by weight, of the composition;
the water is in the composition in an amount between at or about 89.5% and at or about 90.4%, by weight, of the composition;
the potassium sorbate is in the composition in an amount of at or about 0.04%, by weight, of the composition; and
the sodium benzoate is in the composition in an amount of at or about 0.04%, by weight, of the composition.

44. The composition of claim 42, wherein:
the 2-amino-4-methylhexane is in the composition at a concentration of at or about 107 mM;
the flavoring agent is a peach flavoring agent in an amount of at or about 0.663%, by weight, of the composition;
the sucralose is in the composition in an amount of at or about 1.438%, by weight, of the composition;
the caffeine is in the composition in an amount of at or about 2%, by weight, of the composition;
the conjugated linoleic acid (CLA) is in the composition at an amount of at or about 0.248%, by weight, of the composition;
the Vitamin B12 is in the composition in an amount of at or about 0.025%, by weight, of the composition;
the chromium picolinate is in the composition in an amount of at or about 0.015%, by weight, of the composition;
the phosphatidylcholine is in the composition in an amount of at or about 0.075%, by weight, of the composition;
the TPGS surfactant is in the composition in an amount of at or about 0.625%, by weight, of the composition;
the emulsion stabilizer is in the composition in an amount of at or about 0.165%, by weight, of the composition;
the citric acid is in the composition in an amount of at or about 2.66%, by weight, of the composition;
the water is in the composition in an amount of at or about 90.388%, by weight, of the composition;
the potassium sorbate is in the composition in an amount of at or about 0.04%, by weight, of the composition; and
the sodium benzoate is in the composition in an amount of at or about 0.04%, by weight, of the composition.

45. The composition of claim 1, wherein:
the aminoalkane or derivative thereof is 2-amino-4-methylhexane HCl;
the taste-modifying agents comprise sucralose and a mint flavoring agent; and
the composition further comprises: caffeine, conjugated linoleic acid, phosphatidylcholine, a TPGS surfactant, an emulsion stabilizer comprising: guar gum, xanthan gum and sodium alginate, at an amount sufficient to stabilize the composition compared to the absence of the emulsion stabilizer, citric acid, at an amount sufficient to change the pH compared to the composition without the citric acid, water, and potassium sorbate and sodium benzoate, at a combined amount sufficient to preserve the composition compared to the absence of the potassium sorbate and sodium benzoate.

46. The composition of claim 45, wherein:
the 2-amino-4-methylhexane is in the composition at a concentration of at or about 107 mM;
the mint flavoring agent is in the composition at an amount of at or about 1.09%, by weight, of the composition;
the sucralose is in the composition in an amount of at or about 1.44%, by weight, of the composition;
the caffeine is in the composition in an amount of at or about 2%, by weight, of the composition;
the conjugated linoleic acid (CLA) is in the composition in an amount of at or about 0.25%, by weight, of the composition;
the phosphatidylcholine is in the composition in an amount of at or about 0.075%, by weight, of the composition;
the TPGS surfactant is in the composition at an amount at or about 1.25%, by weight, of the composition;
the emulsion stabilizer is in the composition at an amount of at or about 0.165%, by weight, of the composition;
the citric acid is in the composition in an amount of at or about 0.17%, by weight, of the composition;
the water is in the composition in an amount at or about 91.9%, by weight, of the composition;
the potassium sorbate is in the composition in an amount of at or about 0.04%, by weight, of the composition; and
the sodium benzoate is in the composition in an amount of at or about 0.04%, by weight, of the composition.

47. The composition of claim 1, wherein:
the aminoalkane or derivative thereof is 2-amino-4-methylhexane HCl;
the taste-modifying agents comprise sucralose and a spearmint flavoring agent; and
the composition further comprises: caffeine, conjugated linoleic acid, phosphatidylcholine, a TPGS surfactant, an emulsion stabilizer comprising: guar gum, xanthan gum and sodium alginate, at an amount sufficient to stabilize the composition compared to the absence of the emulsion stabilizer, citric acid, at an amount sufficient to change the pH compared to the composition without the citric acid, water, and potassium sorbate and sodium benzoate, at a combined amount sufficient to preserve the composition compared to the absence of the potassium sorbate and sodium benzoate.

48. The composition of claim 47, wherein:
the 2-amino-4-methylhexane is in the composition at a concentration of at or about 107 mM;
the spearmint flavoring agent is in the composition at an amount of at or about 1.5%, by weight, of the composition;
the sucralose is in the composition in an amount of at or about 1.44%, by weight, of the composition;
the caffeine is in the composition in an amount of at or about 2%, by weight, of the composition;
the conjugated linoleic acid (CLA) is in the composition in an amount of at or about 0.25%, by weight, of the composition;
the phosphatidylcholine is in the composition in an amount of at or about 0.075%, by weight, of the composition;
the TPGS surfactant is in the composition at an amount at or about 0.75%, by weight, of the composition;
the emulsion stabilizer is in the composition at an amount of at or about 0.165%, by weight, of the composition;
the citric acid is in the composition in an amount of at or about 2.66%, by weight, of the composition;
the water is in the composition in an amount at or about 89.5%, by weight, of the composition;
the potassium sorbate is in the composition in an amount of at or about 0.04%, by weight, of the composition; and
the sodium benzoate is in the composition in an amount of at or about 0.04%, by weight, of the composition.

49. The composition of claim 1, wherein:
the aminoalkane or derivative thereof is 2-amino-4-methylhexane HCl;
the taste-modifying agents comprise sucralose and a sour apple flavoring agent; and
the composition further comprises: caffeine, conjugated linoleic acid, phosphatidylcholine, a TPGS surfactant, an emulsion stabilizer comprising: guar gum, xanthan gum and sodium alginate, at an amount sufficient to stabilize the composition compared to the absence of the emulsion stabilizer, citric acid, at an amount sufficient to change the pH compared to the composition without the citric acid, water, and potassium sorbate and sodium benzoate, at a combined amount sufficient to preserve the composition compared to the absence of the potassium sorbate and sodium benzoate.

50. The composition of claim 49, wherein:
the 2-amino-4-methylhexane is in the composition at a concentration of at or about 107 mM;
the sour apple flavoring agent is in the composition at an amount of at or about 0.45%, by weight, of the composition;
the sucralose is in the composition in an amount of at or about 1.44%, by weight, of the composition;
the caffeine is in the composition in an amount of at or about 2%, by weight, of the composition;
the conjugated linoleic acid (CLA) is in the composition in an amount of at or about 0.25%, by weight, of the composition;
the phosphatidylcholine is in the composition in an amount of at or about 0.075%, by weight, of the composition;
the TPGS surfactant is in the composition at an amount at or about 0.75%, by weight, of the composition;
the emulsion stabilizer is in the composition at an amount of at or about 0.165%, by weight, of the composition;
the citric acid is in the composition in an amount of at or about 2.66%, by weight, of the composition;
the water is in the composition in an amount at or about 90.515%, by weight, of the composition;
the potassium sorbate is in the composition in an amount of at or about 0.04%, by weight, of the composition; and
the sodium benzoate is in the composition in an amount of at or about 0.04%, by weight, of the composition.

51. The composition of claim 1, wherein:
the aminoalkane or derivative thereof is 2-amino-4-methylhexane HCl;
the taste-modifying agents comprise sucralose and a peppermint or cinnamon flavoring agent; and
the composition further comprises: caffeine, conjugated linoleic acid, phosphatidylcholine, a TPGS surfactant, an emulsion stabilizer comprising: guar gum, xanthan gum and sodium alginate, at an amount sufficient to stabilize the composition compared to the absence of the emulsion stabilizer, citric acid, at an amount sufficient to change the pH compared to the composition without the citric acid, water, and potassium sorbate and sodium benzoate, at a combined amount sufficient to preserve the composition compared to the absence of the potassium sorbate and sodium benzoate.

52. The composition of claim 51, wherein:
the 2-amino-4-methylhexane is in the composition at a concentration of at or about 107 mM;
the peppermint or cinnamon flavoring agent is in the composition at an amount of at or about 1%, by weight, of the composition;
the sucralose is in the composition in an amount of at or about 1.44%, by weight, of the composition;
the caffeine is in the composition in an amount of at or about 2%, by weight, of the composition;
the conjugated linoleic acid (CLA) is in the composition in an amount of at or about 0.25%, by weight, of the composition;
the phosphatidylcholine is in the composition in an amount of at or about 0.075%, by weight, of the composition;
the TPGS surfactant is in the composition at an amount at or about 0.75%, by weight, of the composition;
the emulsion stabilizer is in the composition at an amount of at or about 0.165%, by weight, of the composition;
the citric acid is in the composition in an amount of at or about 2.66%, by weight, of the composition;
the water is in the composition in an amount at or about 90%, by weight, of the composition;
the potassium sorbate is in the composition in an amount of at or about 0.04%, by weight, of the composition; and
the sodium benzoate is in the composition in an amount of at or about 0.04%, by weight, of the composition.

53. The composition of claim 1, wherein:
the aminoalkane or derivative thereof is 2-amino-4-methylhexane HCl;
the taste-modifying agents comprise sucralose and a mint flavoring agent; and
the composition further comprises: caffeine, conjugated linoleic acid, L-taurine, alpha lipoic acid, phosphatidylcholine, a TPGS surfactant, an emulsion stabilizer comprising: guar gum, xanthan gum and sodium alginate, at an amount sufficient to stabilize the composition compared to the absence of the emulsion stabilizer, citric acid, at an amount sufficient to change the pH compared to the composition without the citric acid, water, and potassium sorbate and sodium benzoate, at a combined amount sufficient to preserve the composition compared to the absence of the potassium sorbate and sodium benzoate.

54. A composition of claim 1, comprising
2-amino-4-methylhexane;
a mint flavoring agent;
sucralose;
caffeine;
conjugated linoleic acid (CLA);
L-taurine;
alpha lipoic acid;
phosphatidylcholine;
TPGS; and
a preservative.

55. The composition of claim 1, wherein the composition is in a container.

56. The composition of claim 55, wherein the container selected from among ampoules, vials, bags, tubes, bottles and syringes.

57. The composition of claim 56, wherein the container is a twist-top ampoule.

58. The composition of claim 1, wherein the total volume of the composition is at or about 10 mL, 9 mL, 8 mL, 7 mL, 6 mL, 5 mL, 4 mL, 3 mL, 2 mL or 1 mL.

59. A method for producing a the liquid emulsion composition of claim 1, comprising:
(a) preparing an oil phase by adding oil phase-soluble ingredients to an oil phase vessel, the oil phase ingredients containing a surfactant;
(b) preparing a water phase by adding the water-soluble ingredients including the aminoalkane to a water phase vessel;
(c) combining the water phase and the oil phase;
(d) adding a taste-modifying agent in an amount sufficient to improve or enhance the palatability of the composition compared to the composition in the absence of the agent, thereby producing the palatable liquid composition,
wherein steps (a) and (b) are performed simultaneously, or sequentially in any order.

60. The method of claim 58, wherein:
step (a) further comprises mixing the oil phase ingredients, heating the oil phase ingredients, or a combination thereof.

61. The method of claim 59, wherein step (b) further comprises mixing the water phase ingredients, heating the water phase ingredients, or a combination thereof.

62. A method for providing energy to a subject, comprising administering a composition of claim 1 to a subject.

63. The method of claim 62, wherein administration is oral.

64. The method of claim 62, wherein the composition is contained in an ampoule and delivery is effected by breaking a seal on the ampoule and ingesting the composition contained in the ampoule.

65. The composition of claim 1 that is formulated for single dosage administration.

66. The composition of claim 65, wherein the volume of the composition is between about or is 1-10 ml, 2-9 ml, 3-8 ml, 3-7 ml, 3-5 ml, 3 ml or 4 ml.

67. The composition of claim 54, comprising:
2-amino-4-methylhexane at a concentration of at or about 107 mM;
a mint flavoring agent it an amount of at or about 1.09%, by weight, of the composition;
sucralose is in an amount of at or about 1.44%, by weight, of the composition;
caffeine in an amount of at or about 2%, by weight, of the composition;
conjugated linoleic acid (CLA) in the composition in an amount of at or about 0.25%, by weight, of the composition;
L-taurine in the composition in an amount of at or about 0.25%, by weight, of the composition;
alpha lipoic acid in the composition in an amount of at or about 0.025%, by weight, of the composition;
phosphatidylcholine in the composition in an amount of at or about 0.075%, by weight, of the composition;
TPGS surfactant in the composition at an amount at or about 1.5%, by weight, of the composition;
an emulsion stabilizer in the composition at an amount of at or about 0.165%, by weight, of the composition;
citric acid in the composition in an amount of at or about 0.17%, by weight, of the composition;
potassium sorbate in the composition in an amount of at or about 0.04%, by weight, of the composition;
sodium benzoate in the composition in an amount of at or about 0.04%, by weight, of the composition; and water.
